(19) United States
(12) Patent Application Publication
(10) Pub. No.: US 2025/0359887 A1
(43) Pub. Date: Nov. 27, 2025

(54) SKIN TREATMENT SYSTEMS, DEVICES AND METHODS

(71) Applicant: Cytrellis Biosystems, Inc., Woburn, MA (US)

(72) Inventors: Joshua Matte, Beverly, MA (US); Duncan Silver, Boston, MA (US); Robert Thurman, Billerica, MA (US); Steven Cobb, Westford, MA (US); Monica Salas, Malden, MA (US); Alan Clark, Wakefield, MA (US); Karen Cronholm, Arlington, MA (US); Stephen Gemmell, Andover, MA (US); J. Christopher Flaherty, Nottingham, NH (US)

(21) Appl. No.: 18/695,869

(22) PCT Filed: Sep. 27, 2022
(86) PCT No.: PCT/US22/44862

§ 371 Date: Mar. 27, 2024

Related U.S. Application Data
(60) Provisional application No. 63/248,562, filed on Sep. 27, 2021.
(60) Provisional application No. 63/354,004, filed on Jun. 21, 2022.

(51) Int. Cl. A61B 17/3205; A61B 17/00

(52) U.S. Cl. CPC A61B 17/32053; A61B 2017/00367; A61B 2017/00761

(57) ABSTRACT

Systems for producing a cosmetic effect in skin tissue of a patient are provided. The systems include a treatment module and an actuation assembly. The treatment module includes at least one coring element configured to remove a portion of skin tissue when the coring element is inserted into and withdrawn from the skin tissue. The actuation assembly is operably attached to the treatment module and can translate and/or actuate the treatment module in one or more directions relative to a surface of the skin tissue. The system can perform a microcoring procedure that provides a cosmetic effect to the patient. Methods of performing a microcoring procedure are also provided.

### RELATED APPLICATIONS

This application claims the benefit of U.S. Provisional Patent Application Ser. No. 63/248,562 (Docket No.: CYT-013-PR1), titled “Skin Treatment Systems, Devices and Methods”, filed Sep. 27, 2021, the content of which is incorporated herein by reference in its entirety for all purposes.

This application claims the benefit of U.S. Provisional Patent Application Ser. No. 63/354,004 (Docket No.: CYT-011-PR1), titled “Advanced Skin Treatment Systems and Methods”, filed Jun. 21, 2022, the content of which is incorporated herein by reference in its entirety for all purposes.

This application is related to U.S. patent application Ser. No. 14/764,866 (Docket No.: CYT-001-US), titled “Methods and Devices for Skin Tightening”, filed Jul. 30, 2015, U.S. Pat. No. 10,543,127, issued Jan. 18, 2020, the content of which is incorporated herein by reference in its entirety for all purposes.

This application is related to U.S. patent application Ser. No. 15/905,421 (Docket No.: CYT-001-US-CON1), titled “Methods and Devices for Skin Tightening”, filed Feb. 26, 2018, U.S. Pat. No. 10,251,792, issued Apr. 9, 2019, the content of which is incorporated herein by reference in its entirety for all purposes.

This application is related to U.S. patent application Ser. No. 16/707,122 (Docket No.: CYT-001-US-DIV), titled “Methods and Devices for Skin Tightening”, filed Dec. 9, 2019, Publication No. US 2020/0188184, published Jun. 18, 2020, the content of which is incorporated herein by reference in its entirety for all purposes.

This application is related to U.S. patent application Ser. No. 17/207,172 (Docket No.: CYT-002-US-CON), titled “Microclosures and Related Methods for Skin Treatment”, filed Mar. 19, 2021, United States Publication No. 2021/0322005, published Oct. 21, 2021, the content of which is incorporated herein by reference in its entirety for all purposes.

This application is related to U.S. patent application Ser. No. 14/910,767 (Docket No.: CYT-003-US), titled “Methods and Apparatuses for Skin Treatment using Non-Thermal Tissue Ablation”, filed Feb. 8, 2016, U.S. Pat. No. 10,555,754, issued Feb. 11, 2020, the content of which is incorporated herein by reference in its entirety for all purposes.

This application is related to U.S. patent application Ser. No. 16/722,069 (Docket No.: CYT-003-US-DIV), titled “Methods and Apparatuses for Skin Treatment using Non-Thermal Tissue Ablation”, filed Dec. 20, 2019, United States Publication No. 2020/0121354, published Apr. 23, 2020, the content of which is incorporated herein by reference in its entirety for all purposes.

This application is related to U.S. patent application Ser. No. 15/106,036 (Docket No.: CYT-004-US), titled “Methods and Devices for Manipulating Subdermal Fat”, filed Jun. 17, 2016, U.S. Pat. No. 10,953,143, issued Mar. 23, 2021, the content of which is incorporated herein by reference in its entirety for all purposes.

This application is related to U.S. patent application Ser. No. 17/166,543 (Docket No.: CYT-004-US-DIV), titled “Methods and Devices for Manipulating Subdermal Fat”, filed Feb. 3, 2021, United States Publication No. 2021/0178028, published Jun. 17, 2021, the content of which is incorporated herein by reference in its entirety for all purposes.

This application is related to U.S. patent application Ser. No. 15/526,299 (Docket No.: CYT-005-US), titled “Devices and Methods for Ablation of the Skin”, filed May 11, 2017, U.S. Pat. No. 11,324,534, issued May 10, 2022, the content of which is incorporated herein by reference in its entirety for all purposes.

This application is related to U.S. patent application Ser. No. 17/709,542 (Docket No.: CYT-005-US-CON1), titled “Devices and Methods for Ablation of the Skin”, filed Mar. 31, 2022, U.S. Publication Ser. No. ______, published, ______, the content of which is incorporated herein by reference in its entirety for all purposes.

This application is related to U.S. Design patent application Ser. No. 29/509,219 (Docket No.: CYT-006-DES), titled “Device and Device Body for Mechanical Fractional Ablation of the Skin”, filed Nov. 14, 2014, U.S. Design Pat. No. D797286, issued Sep. 12, 2017, the content of which is incorporated herein by reference in its entirety for all purposes.

This application is related to U.S. patent application Ser. No. 16/090,034 (Docket No.: CYT-007-US), titled “Devices and Methods for Cosmetic Skin Resurfacing”, filed Sep. 28, 2018, U.S. Pat. No. 11,166,743, issued Nov. 9, 2021, the content of which is incorporated herein by reference in its entirety for all purposes.

This application is related to U.S. patent application Ser. No. 17/491,691 (Docket No.: CYT-007-US-CON1), titled “Devices and Methods for Cosmetic Skin Resurfacing”, filed Oct. 1, 2021, United States Publication No. 2022-0125477, published Apr. 28, 2022, the content of which is incorporated herein by reference in its entirety for all purposes.

This application is related to International Patent Application Serial Number PCT/US17/052528 (Docket No.: CYT-008-PCT), titled “Devices and Methods for Cosmetic Skin Resurfacing”, filed Sep. 20, 2017, Publication No. 2018/057630, published Mar. 29, 2018, the content of which is incorporated herein by reference in its entirety for all purposes.

This application is related to U.S. patent application Ser. No. 16/335,028 (Docket No.: CYT-008-US), titled “Devices and Methods for Cosmetic Skin Resurfacing”, filed Mar. 20, 2019, United States Publication No. 2019/0366067, published Dec. 5, 2019, the content of which is incorporated herein by reference in its entirety for all purposes.

This application is related to U.S. patent application Ser. No. 17/902,028 (Docket No.: CYT-008-US-CON1), titled “Devices and Methods for Cosmetic Skin Resurfacing”, filed Sep. 2, 2022, U.S. Publication Ser. No. ______, published ______, the content of which is incorporated herein by reference in its entirety for all purposes.

This application is related to U.S. patent application Ser. No. 16/857,801 (Docket No.: CYT-009-US-CON1), titled “Rapid Skin Treatment Using Microcoring”, filed Apr. 24, 2020, United States Publication No. 2020/0246039, published Aug. 6, 2020, the content of which is incorporated herein by reference in its entirety for all purposes.

This application is related to U.S. patent application Ser. No. 17/291,235 (Docket No.: CYT-010-US), titled “Systems and Methods for Skin Treatment”, May 4, 2021, United States Publication No. 2021/0401453, published Dec. 30, 2021, the content of which is incorporated herein by reference in its entirety for all purposes.

This application is related to U.S. Provisional Patent Application Ser. No. 63/190,904 (Docket No.: CYT-012-PR1), titled “Skin Treatment Systems and Methods”, filed May 20, 2021, the content of which is incorporated herein by reference in its entirety for all purposes.

This application is related to International Patent Application Serial Number PCT/US22/030236 (Docket No.: CYT-012-PCT), titled “Skin Treatment Systems and Methods”, filed May 20, 2022, U.S. Publication Ser. No. ______, published ______, the content of which is incorporated herein by reference in its entirety for all purposes.

### TECHNICAL FIELD

The embodiments disclosed herein relate generally to systems, devices, and methods for treatment of biological tissues.

### BACKGROUND

Many human health issues arise from damage, deterioration, or loss of tissue due to disease, advanced age, and/or injury. These health issues can manifest themselves in a variety of alterations of tissue structure and/or function, including scarring, sclerosis, tightness, and laxity. In aesthetic medicine, elimination of excess tissue and/or skin laxity is an important concern that affects more than 25% of the U.S. population.

### BRIEF SUMMARY

There is a need for improved systems and methods that provide increased effectiveness over currently available techniques while maintaining convenience, affordability, and accessibility to patients desiring tissue restoration.

According to an aspect of the present inventive concepts, a system for producing a cosmetic effect in skin tissue of a patient comprises: a treatment module comprising at least one coring element configured to remove a portion of skin tissue when the coring element is inserted into and withdrawn from the skin tissue; and an actuation assembly operably attached to the treatment module and configured to translate and/or actuate the treatment module in one or more directions relative to a surface of the skin tissue. The system can be configured to perform a microcoring procedure that provides a cosmetic effect to the patient.

In some embodiments, the at least one coring element comprises at least three coring elements. The at least three coring elements can be located at a separation distance of no more than 7 mm, 6 mm, 5 mm, and/or 4 mm.

In some embodiments, the at least one coring element comprises an outer diameter of at least 0.0203″ and/or an outer diameter of no more than 0.0500″.

In some embodiments, the at least one coring element comprises an inner diameter of at least 0.0103″ and/or an inner diameter of no more than 0.0207″.

In some embodiments, the treatment module is configured to detach and operably attach to the actuation assembly.

In some embodiments, the system comprises multiple treatment modules, and each treatment module comprises at least one coring element configured to remove a portion of skin tissue when the coring element is inserted into and withdrawn from the skin tissue, and each treatment module is configured to be operably attached to the actuation assembly.

In some embodiments, the system can further comprise a receiving portion including a handle, and the treatment module is configured to operably attach to the receiving portion. The at least one coring element can be configured to be in a locked state when the treatment module is not attached to the receiving portion. The system can further comprise a sensor configured to produce a signal, and the system can be configured to detect proper attachment of the treatment module to the receiving portion based on the signal. The sensor can comprise a magnetic sensor.

In some embodiments, the actuation assembly comprises a lead screw, and the lead screw comprises: a fine pitched screw, such as a screw with a M3 0.5-6 g thread; a brass screw; and/or a thread engaging component made of plastic, such as PEEK.

In some embodiments, the actuation assembly comprises a translating component and at least one sensor, and the at least one sensor is configured to produce a signal related to a change in position of the translating component, and the system is configured to determine the acceleration, speed, and/or absolute position of the at least one coring element, during advancement and/or retraction of the at least one coring element, based on the signal.

In some embodiments, the actuation assembly comprises a switch configured to detect an end of travel position of the at least one coring element.

In some embodiments, the actuation assembly comprises a switch configured to detect a beginning of travel position of the at least one coring element.

In some embodiments, the actuation assembly comprises a voice coil actuator configured to cause the at least one coring element to translate in a z direction. The actuation assembly can further comprise a sensor configured to produce a signal related to the temperature of the voice coil, and the system can be configured to enter an alert state if the temperature exceeds a threshold.

In some embodiments, the actuation assembly further comprises a spring configured to bias the at least one coring element in a retracted position.

In some embodiments, the system is configured to perform a treatment event at least 3, 8, 12, 17, and/or 20 times, and the treatment event comprises: (1) advancing the at least one coring element into tissue; (2) withdrawing the at least one coring element from tissue; and (3) repositioning the at least one coring element at a new tissue location.

In some embodiments, the system further comprises a single component that is configured to provide: control of one or more motors of the system; control of one or more indicator lights of the system; interface with one or more sensors of the system; and/or inter-component communication for the system. The single component can be configured to provide at least two, three, or all four of: control of one or more motors of the system; control of one or more indicator lights of the system; interface with one or more sensors of the system; and/or inter-component communication for the system.

In some embodiments, the system further comprises a drape and a hand piece shell, and the drape is configured to cover the hand piece shell without adversely affecting movement of the at least one coring element.

In some embodiments, the system is configured to collect patient data. The patient data can comprise data selected from the group consisting of: diagnostic data; patient use data; image data; blood flow data; and combinations thereof.

In some embodiments, the system further comprises an arrangement of one or more housing that surrounds at least the actuation assembly, and the housing arrangement comprises an outer surface, and the arrangement defines a handle portion. The housing arrangement can further define a single opening between the outer surface and components internal to the arrangement. The system can further comprise a shroud attachable to the housing arrangement at a location proximate the single opening, and the shroud can be configured to limit ingress of material into the single opening.

In some embodiments, the system further comprises a vacuum flow pathway fluidly attached to the at least one coring element, and the vacuum flow pathway is configured to remove tissue from the at least one coring element. The vacuum flow pathway can comprise a Y-connector comprising two tubes oriented at an angle less than 90°, 75°, 45°, and/or 30°.

In some embodiments, the system further comprises a flange comprising an opening, and the flange is positioned proximate the at least one coring element, and the flange is configured to be positioned on the patient's skin during microcoring, and the at least one coring element is configured to pass through the opening during microcoring. The flange can be configured to grip the surface of the patient's skin via an applied vacuum. The flange can be configured to provide a metered leak. The flange can comprise a hole, and the hole can be configured to provide the metered leak. The hole can comprise a diameter of at least 0.2 mm, 0.4 mm, 0.6 mm, and/or 0.8 mm. The hole can comprise a diameter of no more than 3 mm, 2.5 mm, 2.0 mm, and/or 1.5 mm. The hole can comprise a taper. The taper can comprise a taper of at least 1° and/or a taper of no more than 5°.

In some embodiments, the system further comprises at least one tissue capture sensor configured to provide a signal related to presence of tissue in the at least one coring element. The at least one tissue capture sensor can comprise at least two sensors, and each sensor can be configured to provide a signal related to presence of tissue in the at least one coring element. The system can be configured to determine proper tissue capture only if both signals indicate proper tissue capture. The system can be configured to determine proper tissue capture if either signal indicates proper tissue capture.

In some embodiments, the system further comprises at least one sensor configured to produce a signal, and the system is configured to monitor the signal and enter an alert state if one, two, or more of the following conditions occur: intended depth of penetration of the at least one microcoring element is not achieved; velocity profile of the at least one microcoring element motion is outside of an intended window; the at least one microcoring element is at an undesired position; acceleration of the at least one microcoring element exceeds a threshold; and/or deceleration of the at least one coring element exceeds a threshold The system can be configured to provide an audible, visual, and/or tactile alarm if an undesired condition can be detected.

In some embodiments, the system further comprises a current monitoring sensor configured to produce a signal related to current flow in one or more components of the system. The system can be configured to detect an undesired condition based on the sensor signal, and the undesired condition can comprise a condition selected from the group consisting of: the actuation assembly having to exert an undesired amount of force; an actuation assembly component being in a stuck position; the actuation assembly being in a locked state; the treatment module being improperly attached to the actuation assembly; and combinations thereof.

In some embodiments, the system is configured to monitor repeated use of the treatment module.

In some embodiments, the system further comprises a controller and a memory storage component coupled to the controller, and the memory storage component stores instructions for the controller to perform an algorithm. The algorithm can comprise an AI algorithm. The system can further comprise a calibration device including one or more sensors, and each sensor produces a signal related to the position of one or more movable portions of the actuation assembly, and the algorithm can be configured to calibrate the actuation assembly via the signals of the one or more sensors. The one or more sensors can comprise at least two sensors selected from the group consisting of: optical sensor; magnetic sensor; force sensor; sound sensor such as ultrasound sensor; density sensor; and combinations thereof. The algorithm can be configured to limit the depth of insertion of the at least one coring element. The limiting of the depth of insertion can be configured to avoid the at least one coring element contacting a nerve, a blood vessel, and/or bone. The algorithm can be configured to control the acceleration and/or deceleration of the at least one coring element. The actuation assembly can comprise at least one encoder and/or at least one other sensor that is configured to produce a signal related to the motion of the at least one coring element, and the algorithm controls the depth of insertion based on the signal. The at least one encoder and/or at least one other sensor can comprise at least one absolute position encoder. The system can be configured to control motion of the at least one coring element with a resolution of no more than 5 μm, 4 μm, 3 μm, 2 μm, and/or 1 μm. The system can be configured to control motion of the at least one coring element in the x direction, y direction and/or z direction. The system can be configured to control motion in at least two directions, and/or at least three directions. The algorithm can be configured to control and/or adjust the depth of insertion of the at least one coring element. The system can further comprise at least one sensor configured to produce a signal related to deceleration of the at least one coring element, and the algorithm can be configured to perform the controlling and/or adjusting of the depth of insertion based on the signal. The algorithm can be configured to detect when acceleration of the at least one coring element exceeds a threshold. The algorithm can be configured to detect when deceleration of the at least one coring element exceeds a first threshold. The first threshold can comprise a threshold of 75 g, 60 g, and/or 50 g. The algorithm can be configured to detect when multiple decelerations of the at least one coring element each exceed a second threshold, and the second threshold is less than the first threshold. The algorithm can be configured to detect inadequate communication between two or more components of the system.

In some embodiments, the system further comprises at least one redundant component configured to easily replace another component of the system.

In some embodiments, the system further comprises a console that operably attaches to the actuation assembly. The console can comprise a user interface.

In some embodiments, the system further comprises a tissue removal tool configured to remove tissue from the at least one coring element. The tissue removal tool can comprise a cloth, an insertable filament, and/or a vacuum-based tool.

In some embodiments, the system further comprises a manufacturing tool configured to manufacture the treatment module. The at least one coring element can comprise at least two coring elements, and the tool can be configured to manufacture the at least two coring elements in a desired geometric orientation in the treatment module. Each coring element can comprise a distal end, and the tool can be configured to avoid damaging the distal end of each coring element during the manufacture of the treatment module. The tool can be configured to axially and/or rotationally position each coring element in the treatment module during the manufacture of the treatment module.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a block diagram of a system for treating and/or diagnosing tissue, consistent with the present inventive concepts.

FIG. 2 illustrates a side view of a coring element being introduced into the skin, consistent with the present inventive concepts.

FIG. 3A, FIG. 3B, FIG. 3C, and FIG. 3D illustrate end, side, and sectional views of a needle, consistent with the present inventive concepts.

FIG. 4 illustrates a block diagram of a system for treating and/or diagnosing tissue, consistent with the present inventive concepts.

FIG. 5 illustrates a cut-away view of an example apparatus for microcoring, consistent with the present inventive concepts.

FIG. 6 illustrates a cut-away view of an example apparatus for microcoring, consistent with the present inventive concepts.

FIG. 7 illustrates a perspective cutaway view of an example apparatus for microcoring, consistent with the present inventive concepts.

FIG. 8 illustrates a perspective view of an example apparatus for microcoring, consistent with the present inventive concepts.

FIG. 9 illustrates a perspective view of an example actuation unit, consistent with the present inventive concepts.

FIG. 9A illustrates a cut-away view of an example needle inserted in skin tissue, consistent with the present inventive concepts.

FIG. 10 illustrates an example plot of voice coil velocity, position, and acceleration against time during an example normal coring procedure, consistent with the present inventive concepts.

FIG. 11 illustrates an example plot of voice coil velocity, position, and acceleration against time before, during, and after a coring procedure with excessive over penetration and contact with hard tissue, consistent with the present inventive concepts.

FIG. 12 illustrates a cut-away view diagram of an example apparatus with an example mechanism, e.g. to raise or lower (e.g. relative to a skin surface during operation) a z-actuator, consistent with the present inventive concepts.

FIG. 13A illustrates a cut-away view of an example needle hub with one coring needle, consistent with the present inventive concepts.

FIG. 13B illustrates a semi-transparent view of an example needle hub with one coring needle, consistent with the present inventive concepts.

FIG. 13C illustrates a diagram illustrating an example core clearing procedure in an example needle hub with one coring needle, consistent with the present inventive concepts.

FIG. 14 illustrates example results of a computational fluid dynamics simulation of fluid flow in an example channel of an example needle hub, consistent with the present inventive concepts. Arrows indicate flow direction. Gray scale of arrows indicates Mach number.

FIG. 15 illustrates example results of a computational fluid dynamics simulation of fluid flow in an example channel of an example needle hub, consistent with the present inventive concepts. Gray scale indicates fluid pressure.

FIG. 16 illustrates a perspective exploded view of components of an example needle hub and core clearing system with three needles, consistent with the present inventive concepts.

FIG. 17A and FIG. 17B illustrate cross-sectional views of an example needle hub for three needles, consistent with the present inventive concepts.

FIG. 17C, FIG. 17D and FIG. 17E illustrate perspective views of an example needle hub for three needles, consistent with the present inventive concepts.

FIG. 18A illustrates a cross-sectional view of an example needle hub insert, consistent with the present inventive concepts.

FIG. 18B and FIG. 18C illustrate perspective views of an example needle hub insert, consistent with the present inventive concepts.

FIG. 19 illustrates a perspective view of components of an example needle hub and core clearing system with three needles, consistent with the present inventive concepts.

FIG. 20 illustrates a semi-transparent cut-away view of an example needle hub with three needles, consistent with the present inventive concepts.

FIG. 21A illustrates a perspective view of an example needle hub with an example hub shield, consistent with the present inventive concepts.

FIG. 21B illustrates a cross-sectional view of an example needle hub with an example hub shield.

FIG. 21C illustrates a side view of an example needle hub with an example hub shield, consistent with the present inventive concepts.

FIG. 22 illustrates a perspective view of an example needle hub with an example hub shield and an example spacer, consistent with the present inventive concepts.

FIG. 23 illustrates a perspective exploded view of components of an example needle hub and core clearing system with one needle, consistent with the present inventive concepts.

FIG. 24A illustrates a cross-sectional view of an example needle hub for one needle, consistent with the present inventive concepts.

FIG. 24B illustrates an enlargement of the encircled portion of FIG. 24A, consistent with the present inventive concepts.

FIG. 24C, FIG. 24D, and FIG. 24E illustrate perspective views of an example needle hub for one needle, consistent with the present inventive concepts.

FIG. 25A, FIG. 25B, and FIG. 25C illustrate perspective views of an example vacuum spacer, consistent with the present inventive concepts.

FIG. 26A, and FIG. 26B illustrate perspective views of an example vacuum spacer, consistent with the present inventive concepts.

FIG. 27A illustrates a cross-sectional view of an example vacuum spacer, consistent with the present inventive concepts.

FIG. 27B illustrates an enlargement of the circled portion of FIG. 27A, consistent with the present inventive concepts.

FIG. 28 illustrates a perspective view of an example vacuum spacer system, consistent with the present inventive concepts.

FIG. 29 illustrates a perspective view of an example vacuum spacer frame, consistent with the present inventive concepts.

FIG. 30 illustrates a diagram of an example low pressure or (partial) vacuum system, consistent with the present inventive concepts.

FIG. 31 illustrates a perspective view of possible needle prong configurations for a hollow needle, consistent with the present inventive concepts.

FIG. 32 illustrates a schematic showing a side view of a prong of a hollow needle, consistent with the present inventive concepts. A bevel angle a of a prong refers to the angle between lateral side of the prong and longitudinal axis of the hollow needle.

FIG. 33 illustrates photographs that compare needle heel degradations after 2,000, 8,000, and 10,000 actuation cycles of hollow needles having a bevel angle of 10 degrees, 20 degrees, or 30 degrees, consistent with the present inventive concepts.

FIG. 34 illustrates a schematic showing needle coring force and tissue resistance force on a cored tissue portion inside the lumen of an example hollow needle, consistent with the present inventive concepts.

FIG. 35 illustrates a circuit schematic of a tissue treatment system, consistent with the present inventive concepts.

FIG. 36 illustrates a side view of a handheld device with a portion of a housing removed, consistent with the present inventive concepts.

FIG. 37 illustrates a side view of a handheld device positioned relative to a patient's face, consistent with the present inventive concepts.

FIGS. 38A-C illustrate a perspective view, a side view, and a perspective view, respectively, of a portion of a treatment device, consistent with the present inventive concepts.

FIG. 38D is a side view of a portion of a treatment device, consistent with the present inventive concepts.

FIGS. 38E-F are side sectional views of a portion of a treatment device, consistent with the present inventive concepts.

FIGS. 39A and 39B illustrate a partially transparent and a cross-sectional side view of a portion of a treatment device, respectively, consistent with the present inventive concepts.

FIGS. 40A-D illustrate a side view, and three side sectional views of an actuator of a treatment device, consistent with the present inventive concepts.

FIGS. 41A-B illustrate two side views of an actuation assembly of a treatment device, consistent with the present inventive concepts.

FIG. 42 illustrates a perspective view of a manufacturing tool, consistent with the present inventive concepts.

### DETAILED DESCRIPTION OF THE DRAWINGS

Reference will now be made in detail to the present embodiments of the systems, devices, and methods (singly or collectively “technology” or “technologies” herein), examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

As used herein, the term “operably attaches”, and similar terms related to attachment of components shall refer to attachment of two or more components that results in one, two, or more of: electrical attachment; fluid attachment; magnetic attachment; mechanical attachment; optical attachment; acoustic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereinabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “conduit” or “conduits” can refer to an elongate component that can include one or more flexible and/or non-flexible filaments selected from the group consisting of: one, two or more wires or other electrical conductors (e.g. including an outer insulator); one, two or more wave guides; one, two or more hollow tubes, such as hydraulic, pneumatic, and/or other fluid delivery tubes; one or more optical fibers; one, two or more control cables and/or other mechanical linkages; one, two or more flex circuits; and combinations of these. A conduit can include a tube including multiple conduits positioned within the tube. A conduit can be configured to electrically, fluidically, sonically, optically, mechanically, and/or otherwise operably connect one component to another component.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb); sound (e.g. a transducer comprising one or more piezoelectric and/or CMUT transducers configured to deliver and/or receive ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising one or more piezoelectric and/or CMUT transducers); chemical energy; electromagnetic energy; magnetic energy; and combinations of two or more of these. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy to tissue, such as to treat and/or image tissue. In some embodiments, a functional element comprises one or more hollow filaments (e.g. one or more needles) that are configured to be inserted into tissue and/or withdrawn from tissue, such as to perform a microcoring treatment as described herein. In some embodiments, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. perform a microcoring procedure, deliver therapeutic energy, and/or deliver a therapeutic agent). In some embodiments, a functional element comprises one or more elements constructed and arranged to perform a function selected from the group consisting of: core and/or remove tissue; deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A “functional assembly” can comprise an assembly constructed and arranged to perform a function, such as are described hereinabove. In some embodiments, a functional assembly is configured to core tissue and/or otherwise treat tissue (e.g. a functional assembly configured as a treatment assembly or treatment module). Alternatively or additionally, a functional assembly can be configured as a diagnostic assembly that records one or more parameters, such as a patient physiologic parameter; a patient anatomical parameter; a patient environment parameter; and/or a system parameter. A functional assembly can comprise a deployable assembly, such as a robotically controlled assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

As used herein, the term “target tissue” comprises one or more volumes of tissue of a patient to be diagnosed and/or treated. Similarly, a “treatment target” or “tissue target” comprises one or more volumes of tissue to be diagnosed and/or treated. “Safety margin tissue” comprises tissue whose treatment (e.g. receiving of a microcoring treatment) yields no significant adverse effect to the patient. “Non-target tissue” comprises tissue that is not intended to receive treatment (e.g. not intended to receive a microcoring treatment).

As used herein, the term “system parameter” comprises one or more parameters of the system of the present inventive concepts. A system parameter can comprise one or more “tissue treatment parameters” (also referred to as “tissue treatment settings”), such as one, two or more tissue treatment parameters selected from the group consisting of: a “microcoring parameter” (also referred to as a “coring parameter” herein); a target level of a patient parameter such as a patient diagnostic parameter and/or a patient environment parameter as described herein; a tissue-type parameter; a tissue target area parameter; a tissue anatomical location area parameter; and combinations of these. Microcoring parameters include but are not limited to: depth of penetration of a coring element; duration and/or speed of penetration of a coring element such as rise time of speed of penetration of a coring element; penetration dwell time (also referred to as “hold time”); duration and/or speed of withdrawal of a coring element; time between penetrations; density of coring (also referred to as “microcoring density”); spacing between coring elements; coring diameter; location of penetration; coring suction force; skin suction force (e.g. vacuum pressure and contact area); vacuum “pinch” time (e.g. time to release skin suction); vacuum regeneration time (e.g. as dictated by tubing and/or filter volume and controlled leaks in the system); frequency of coring; inner diameter surface friction of coring element; and combinations of these. A system parameter can comprise a parameter selected from the group consisting of: a tissue treatment parameter; a microcoring parameter; an energy delivery parameter; a pressure level; a temperature level; an energy level; a power level; a frequency level; an amplitude level; a battery level; a threshold level for an alarm or other alert condition; and combinations of these. A system parameter can include one or more tissue targets identified to be treated (e.g. areas of skin tissue to be treated), such as tissue targets identified for treatment by an operator and/or by an algorithm of the system.

As used herein, the term “patient parameter” comprises one or more parameters associated with the patient. A patient parameter can comprise a patient physiologic parameter, such as a physiologic parameter selected from the group consisting of: temperature (e.g. tissue temperature); pressure such as blood pressure or other body fluid pressure; pH; a blood gas parameter; blood glucose level; hormone level; heart rate; respiration rate; and combinations of these. Alternatively or additionally, a patient parameter can comprise a patient environment parameter, such as an environment parameter selected from the group consisting of: patient geographic location; temperature; pressure; humidity level; light level; time of day; and combinations of these.

As used herein, the term “image data” comprises data created by one or more imaging devices. Image data can include data related to target tissue, safety margin tissue, and non-target tissue. Image data can also include data related to any implants or other non-tissue objects that are proximate tissue being imaged. Image data can be processed by one or more algorithms of the present inventive concepts, such as to determine one or more locations to treat (e.g. target tissue identified to be ablated or otherwise receive microcoring or other treatment), and/or to determine one or more locations to which treatment (e.g. microcoring) is to be avoided (e.g. non-target tissue). Image data can comprise data produced by a single imaging component, or from multiple imaging components.

As used herein, the term “transmitting a signal” and its derivatives shall refer to the transmission of power and/or data between two or more components, in any direction, such as via wired or wireless connections.

As used herein, the term “patient use data” shall refer to data related to use of the tissue treatment systems of the present inventive concepts on a patient (e.g. use of the system in a diagnostic and/or therapeutic procedure performed on a patient). The data can include but is not limited to: operating parameters such as tissue treatment parameters; target tissue parameters such as location of target tissue and/or amount of target tissue to be treated; patient parameters such as patient physiologic parameters and/or patient location or other patient environment parameters; clinician parameters; clinical site parameters; and combinations of these. Patient use data can include data from multiple patients, such as data collected from multiple patients that interface with (e.g. receive a treatment from) one or more systems of the present inventive concepts. In some embodiments, an algorithm of the present inventive concepts uses patient use data from one or more patients to determine a system parameter to be used in performing a medical procedure on a patient.

The systems, devices, and methods of the present inventive concepts can be configured for treating skin (e.g. eliminating tissue volume, tightening skin, lifting skin, reducing skin laxity, and/or otherwise providing a cosmetic effect), such as by selectively excising a plurality of microcores of patient tissue. In some embodiments, the tissue is treated without thermal energy being imparted to surrounding (e.g. non-excised) tissue. These systems, devices, and methods satisfy an unmet need for rapid and safe treatment of skin (“skin” or “skin tissue” herein), including, for example, faster pretreatment preparation and post-treatment healing times as compared to current surgical and thermal treatment methods.

In general, the term “microcoring,” as used herein, refers to technologies that utilize one or more (in some embodiments, a plurality of, e.g. an array of) hollow needles, and/or other non-thermal treatment elements (e.g. blades, tubes, and/or drills) that remove and/or otherwise treat tissue of a patient. These treatment elements can be of sufficiently small dimension (e.g. comprise a sufficiently small diameter) such as to minimize the extent of bleeding and/or clotting within holes or slits, and/or to minimize scar formation, when used to excise (e.g. and optionally sequester) tissue from a site. In some embodiments, excising a tissue means forming a tissue portion (e.g. a “microcore”), such as by inserting a hollow needle into the site so that the tissue portion is formed inside the hollow needle and severed from surrounding tissue, whereby a microcore that is separated (e.g. physically separated) from other tissue is generated.

In some embodiments, microcoring elements, assemblies, and/or other components as described herein may include a component configured to perform sequestration of the excised tissue. As used herein, the term “sequestering”, when used in reference to tissue, means excising a microcore and then removing the excised microcore from the excision site. In certain embodiments, sequestered tissue may be permanently disposed. In certain embodiments, sequestered tissue may be used for diagnostic purpose, such as when used for biopsy and/or histology analyses, such as those known in the art. In some embodiments, technologies provided herein maximize removal and/or minimize risk of (partial or complete) re-insertion of extracted tissue.

It should be understood that particular microcoring technologies using hollow needles specifically described herein serve for exemplary and/or illustrative purposes, and that other techniques and devices can be used to create microcores. Microcoring technologies described herein may include a number of advantageous features. For example, provided technologies may enable visualization of results in real time during the course of the treatment, such as through feedback (e.g. patient and/or clinician feedback) and subsequent treatment adjustment in real time.

Alternatively or additionally, the systems and devices of the present inventive concepts that are used for microcoring can include micro-sized features that may be beneficial for controlling extent of skin treatment and/or minimize adverse effects of the skin treatment.

Still further, in some embodiments, technologies described herein may require less skill than that of a surgeon. Thus, in certain embodiments, a patient may be treated by a non-physician professional and/or in an outpatient setting, rather than in an inpatient, surgical setting. In some embodiments, a patient may be treated at a spa, at a cosmetic salon, or at home. That is, the technologies of the present inventive concepts are amenable to and/or permit consistent and/or reproducible administration of skin treatment procedures in a variety of treatment settings, and with a broad range of clinicians, technicians, and/or other operators (“operators” or “users” herein) performing the procedures.

In some embodiments, the technologies described herein may have generally a lower risk profile and/or the technologies can provide more predictable results and/or risk factors than those for more invasive techniques (e.g. plastic surgery) or energy-based techniques (e.g. laser, radiofrequency (RF), or ultrasound), which may or may not be invasive.

In some embodiments, non-thermal fractional excision technologies described herein allow skin tightening, skin lifting, and/or reduction of skin laxity without (or with significant reduction of) one or more common side effects of thermal treatment methods (e.g. thermal ablation and/or other treatment methods that increase and/or otherwise modify the temperature of tissue in order to provide a treatment to that tissue). Thermal ablation techniques prevent and/or inhibit skin tightening by allowing coagulation of tissue and formation of rigid tissue cores that cannot be compressed. Thermal ablation techniques create a three-dimensional heat-affected zone (HAZ) surrounding an immediate treatment site. While fractional ablative lasers may be used on or near heat-sensitive sites (e.g. eyes, nerves), for example when the laser does not penetrate more than 1 mm into the skin (resulting in a comparatively small HAZ), other thermal ablation techniques (e.g. ultrasound-based techniques and radiofrequency-based techniques) cannot be used in the vicinity of heat-sensitive sites because the HAZ may extend to heat sensitive tissues potentially causing undesired damage (e.g. permanent undesired damage). As will be appreciated by those skilled in the art, a “heat-sensitive site” is a site where exposure to radiation and/or elevated temperature is associated with a relatively high risk of unacceptable cosmetic and/or physiologic outcomes. In any event, technologies of the present inventive concepts described herein have generally a lower risk profile than, for example, thermal methods, at least in part due to a zone of tissue injury that is smaller than the zone of injury (e.g. the HAZ) of thermal methods.

In some embodiments, advantages of certain technologies described herein include a therapeutic benefit selected from the group consisting of: a particularly low (e.g. lesser than that observed with other techniques such as invasive techniques and/or thermal techniques) degree of erythema; faster resolution of erythema; lower percent incidence, severity, and/or term of skin discoloration (hyperpigmentation or hypopigmentation); low swelling and/or inflammation (e.g. as compared, with that observed with laser treatment and/or with ultrasound-based treatment); and combinations of these.

In some embodiments, the technologies provided herein can allow for rapid closing of holes and/or slits after excising tissue (e.g. within a few seconds after treating skin, such as within ten seconds), thereby minimizing extent of bleeding and/or clotting within holes and/or slits, and/or minimizing the extent of scar formation.

In some embodiments, the technologies provided herein may be useful for maximizing treatment effect while minimizing treatment time, such as by using rapid-fire reciprocating needles or needle arrays, and/or by using large needle arrays that allow for simultaneous excision of tens, hundreds, or even thousands of microcores.

In some embodiments, the technologies described herein may be useful for maximizing tightening effect while minimizing healing time and/or minimizing the time in which a cosmetic effect occurs, such as by optimizing tightening (e.g. by controlling the extent of skin pleating, such as by increasing the extent of skin pleating for some applications or skin regions and/or by decreasing the extent of skin pleating for other applications or skin regions, as described herein).

In some embodiments, the technologies described herein may provide efficient clearance of sequestered and/or partially ablated tissue, and/or provide efficient clearance of debris from ablated tissue portions, thus reducing time for healing and/or improving the skin tightening treatment (e.g. relative to laser-based and/or other thermal technologies).

In some embodiments, the technologies described herein may be configured to allow for efficient and effective positioning of skin prior to, during, and/or after tissue excision (e.g. excision including tissue sequestration). Positioning the skin can be critical to control skin-tightening direction, and it can ensure treatment occurs in the desired location and desired dimensions (e.g. thickness, width in a preferred direction, such as along or orthogonal to Langer lines).

Among other things, the systems, devices, and methods of the present inventive concepts can include microcoring technologies that are configured to achieve desirable (e.g. reduced) procedure times and/or can significantly improve one or more aspects of healing from a tissue treatment procedure (e.g. a tissue removal procedure), such as when compared to thermal methods.

Described herein are technologies, methods, and/or devices for treating skin, such as by selectively microcoring skin tissue. In particular, described herein are hollow needles or other hollow filaments (“needles” herein), as well as related systems (e.g. including kits), devices, and methods, capable of microcoring tissue portions by capturing and retaining the tissue portions inside a lumen of one or more hollow needles after insertion into and withdrawal from the skin. Microcored tissue portions can be removed from a lumen of a hollow needle and discarded. The process can be repeated to generate multiple microcored (also referred to as “cored” herein) skin tissue portions, in particular over a desired area of skin and located at chosen sites of the body of a patient. The hollow needles, kits, devices, methods, and other technologies described herein may provide increased effectiveness over currently available apparatuses and techniques while maintaining convenience, affordability, and accessibility to patients desiring tissue restoration.

In some embodiments, technologies described herein include a treatment device, such as a handheld treatment device. An example treatment device may include a treatment module (e.g. a needle hub) comprising at least one hollow needle configured to remove a portion of the skin tissue (e.g. a microcore) when the hollow needle is inserted into and withdrawn from the skin tissue. In some embodiments, a treatment device may include an activation assembly (e.g. a translation and/or actuation assembly) connected to the treatment module, such as to translate (e.g. along one, two, and/or three axes) and/or actuate the treatment module in one or more directions relative to a surface of the skin tissue. In some embodiments, a treatment device may include a spacer to stabilize and/or maintain a constant position of the treatment device relative to the surface of the patient's skin tissue. In some embodiments, a treatment device may include a hand piece including a hand piece shell, such as a housing that at least partially encases the activation assembly. In some embodiments, a hand piece and/or hand piece shell may include or may be connected to a spacer, such as a connection at a distal end of a treatment device (e.g. an end of a treatment device for contacting skin).

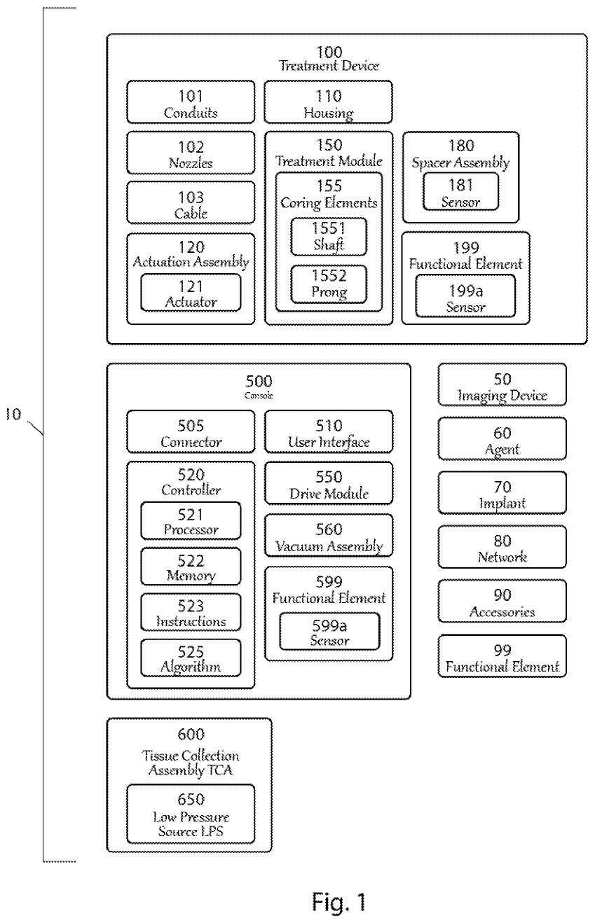

Referring now to FIG. 1, a schematic view of a tissue treatment system is illustrated, consistent with the present inventive concepts. System 10 can be configured to perform a medical procedure on a patient. A medical procedure performed using system 10 can include the performance of one or more clinical procedures, such as one or more diagnostic procedures and/or one or more treatment procedures (e.g. a tissue treatment procedure) performed on a patient. In some embodiments, system 10 is used by an operator (e.g. a clinician, technician, and/or other operator) to perform one, two or more clinical procedures, that are performed within a single day or over multiple days. System 10 can be configured to diagnose and/or treat one or more medical conditions (e.g. diseases, disorders, and/or cosmetic issues) of the patient. System 10 can be configured to treat and/or diagnose one or more portions (e.g. volumes) of patient tissue, “target tissue” herein. In some embodiments, system 10 comprises one, two or more devices that are configured to treat target tissue, such as to improve cosmesis of the patient (e.g. via microcoring as described herein). In some embodiments, system 10 is of similar construction and arrangement, and can include similar components, to the systems described in applicant's co-pending U.S. patent application Ser. No. 17/291,235, titled “Systems and Methods for Skin Treatment”, May 4, 2021.

System 10 can include one or more devices that are configured to record, store, measure, and/or otherwise collect (singly or collectively, “collect”, “record” or “measure” herein) patient data, patient data PD herein. For example, system 10 can include one or more devices or other components configured to collect patient data PD comprising patient diagnostic data, diagnostic data DD. Diagnostic data DD can comprise data related to a physiologic parameter of the patient, data related to the anatomy of the patient, data related to the environment of the patient (e.g. the current environment of the patient), and/or other patient-related data. Alternatively or additionally system 10 can include one or more devices or other components configured to collect patient use data (e.g. as defined herein). Alternatively or additionally, system 10 can include one or more devices or other components configured to collect patient data PD comprising patient image data, image data ID, which can comprise image data of tissue and/or one or more objects proximate tissue. Patient data PD can include data that is used in determining (e.g. by system 10 and/or an operator of system 10) a diagnosis and/or prognosis (either or both, “diagnosis” herein) for the patient. Alternatively or additionally, patient data PD can include patient data that is used in a tissue treatment procedure (e.g. by system 10 and/or an operator of system 10), such as to guide or otherwise affect a microcoring and/or other treatment performed on the patient. Image data ID can include image data related to: target tissue; safety margin tissue; non-target tissue; an implanted diagnostic and/or a treatment device; a foreign body (e.g. a splinter, tattoo, and the like); and combinations of these. System 10 can be configured to produce image data ID through the delivery of energy, such as X-ray energy, sound energy (e.g. ultrasound energy), and/or light energy that is delivered and whose reflections and/or other transmissions are collected in order to produce image data ID. In some embodiments, image data ID comprises data related to tissue comprising blood, such as when image data ID comprises blood flow data (e.g. as obtained using Doppler ultrasound).

As used herein, a “tissue diagnostic procedure”, a “tissue diagnostic”, and their derivatives include but are not limited to: collection of diagnostic data DD; collection of image data ID (e.g. when system 10 records reflections and/or other transmissions of delivered X-ray, ultrasound, light, and/or other energy, and converts these recordings into image data ID); delivery of energy to tissue to characterize the tissue (e.g. when system 10 records one or more effects on the tissue due to the energy delivery, such as using spectroscopy); and/or recording of one or more tissue properties using one or more sensors and/or imaging devices of system 10. A tissue diagnostic procedure can also include a procedure in which various patient parameters are collected, such as patient environment parameters and/or a patient physiologic parameter, for example as described herein.

As used herein, a “tissue treatment procedure”, a “tissue treatment”, and their derivatives include but are not limited to: microcoring of tissue; removal of tissue; ablation of tissue; causing the necrosis of tissue; reducing the volume of tissue (e.g. debulking tissue); stimulating tissue; improving the strength of tissue (e.g. muscle tissue); manipulating and/or otherwise applying a force to tissue; stiffening tissue; and/or otherwise providing a cosmetic enhancement and/or other therapeutic effect to tissue.

System 10 includes treatment device 100 which can comprise one, two or more treatment devices that are configured to perform a treatment procedure on a patient (e.g. a microcoring or other tissue treatment procedure). Treatment device 100 can be configured to treat target tissue (e.g. perform a microcoring of target tissue). Alternatively or additionally, treatment device 100 can be configured to diagnose target tissue (e.g. gather diagnostic data DD and/or image data ID). Treatment device 100 can include one or more modules, treatment module 150 shown, each of which can be configured to perform a patient treatment (e.g. a microcoring treatment). Treatment module 150 can comprise one, two, three or more filaments for coring tissue, coring elements 155 shown. Treatment device 100 can include actuation assembly 120 shown, which can comprise one, two or more assemblies configured to interface with treatment module 150, such as is described herein. Treatment device 100 can include spacer assembly 180 shown, which can comprise one or more assemblies that are constructed and arranged to be positioned between a corresponding one or more treatment modules 150 and tissue.

System 10 can include console 500 shown, which can comprise one, two or more discrete devices, where each of which can operably attach to one, two or more treatment devices 100, simultaneously and/or sequentially. Console 500 can include a connector, connector 505 as shown, which can be configured to operably attach (e.g. electrically, mechanically, fluidly, optically, sonically, and/or otherwise operably attach) to treatment device 100, such as via cable 103 of treatment device 100. Console 500 can be configured to allow an operator to control one or more treatment devices, such as via user interface 510 shown. Console 500 can comprise various assemblies and other components, as described herein, which singly or in combination are configured to provide to treatment device 100 one or more of: energy; mechanical, hydraulic, and/or pneumatic linkages; an agent (e.g. agent 60 described hereinbelow); and/or control signals. Console 500 can be configured to receive data from treatment device 100. In some embodiments, all or a portion of a console 500 is integrated into a treatment device 100 (e.g. the treatment device 100 is a relatively stand-alone device). Console 500 can comprise one or more algorithms, algorithm 525 shown. In some embodiments, treatment device 100 and/or another component of system 10 comprises all or a portion of algorithm 525.

System 10 can include imaging device 50 shown, which can comprise one, two or more imaging devices. Imaging device 50 can be configured to collect image data ID. In some embodiments, imaging device 50 comprises one, two or more imaging devices selected from the group consisting of: a fluoroscope or other X-ray imaging device; an ultrasound imager; a CT scanner; an MRI; an OCT imaging device; a camera such as a visual light camera and/or an infrared camera; and combinations of these. Imaging device 50 can comprise a device configured to characterize and/or otherwise collect data related to one or more properties of tissue, such as a device (e.g. an ultrasound-based device) configured to measure elasticity of tissue and/or other tissue property (e.g. with or without collecting an image of the tissue). In some embodiments, image data ID provided by imaging device 50 can be used to determine a target area to treat with system 10, and/or a non-target area to which treatment should be avoided. For example, algorithm 525 can be configured to analyze image data ID and provide feedback (e.g. suggestions and/or requirements) for particular tissue areas to be classified as target areas and/or non-target areas. In some embodiments, algorithm 525 is configured to identify one or more implants or other objects present under the patient's skin, to which treatment should be adjusted (e.g. avoided), such as an under-the-skin object comprising: a medical implant (e.g. implant 70 described hereinbelow) such as a cosmetic implant; a splinter; and/or tattoo ink. In these embodiments, algorithm 525 can be configured to identify a periphery of the under-the-skin object, such as to define a non-target zone including at least the area within the periphery (e.g. and also including a safety margin outside of the periphery).

System 10 can include agent 60 shown, which can comprise one or more pharmaceuticals and/or other agents that can be delivered to the patient. Agent 60 can comprise an agent that is applied topically and/or an agent that is delivered systemically (e.g. orally). Agent 60 can comprise one, two, or more agents selected from the group consisting of: hyaluronic acid; a moisturizer; an analgesic; a peptide; platelet rich plasma (PRP); arnica montana extract; a vasoconstrictor; methotrexate; minoxidil; stem cells; botulinum toxin; a corticosteroid; and combinations of these. Agent 60 can comprise an agent that is applied topically, and or inserted into the patient, such as into the dermis of the patient, such as when deposited in or otherwise proximate one or more target areas to be treated (e.g. pre-microcoring), during treatment (e.g. when deposited via coring elements 155 or otherwise), and/or after treatment (e.g. after microcoring). In some embodiments, a functional element 99 (e.g. as described hereinbelow) comprises a delivery device configured to deliver agent 60, such as a syringe, needle, transdermal patch, microfluidic pump, and/or other delivery device configured to deliver agent 60 to the surface of the skin and/or to an internal location (e.g. into the dermis).

System 10 can include implant 70 shown, which can comprise one or more implants which can be implanted in the patient such as to improve cosmesis of the patient, and/or to treat a disease and/or disorder of the patient. In some embodiments, a treatment performed by system 10 includes the implantation of one or more implants 70, such as to further improve cosmesis of the patient. In some embodiments, a treatment performed by system 10 is adjusted due to the presence of an existing implant (e.g. implant 70), and/or due to a future implantation of an implant (e.g. implant 70).

System 10 can include tissue collection assembly 600 shown (also referred to as “TCA 600” herein), which can comprise one or more assemblies configured to collect tissue which has been removed from the patient by treatment module 150. TCA 600 can comprise one or more containers for storing collected tissue. TCA 600 can comprise a vacuum pump and/or other low-pressure source, LPS 650 shown, such as to create a pressure differential which causes tissue extracted by treatment device 100 to be drawn into TCA 600.

System 10 can include one or more functional elements, such as functional element 199 of treatment device 100, and/or functional element 599 of console 500, and/or functional element 99, each as shown. Functional elements 99, 199, and/or 599 can comprise one or more sensors and/or transducers, and/or an assembly that includes one or more sensors and/or transducers. Functional element 99, 199, and/or 599 can comprise a component (e.g. a sensor, or an assembly including a sensor) that is configured to collect patient data PD, such as diagnostic data DD and/or image data ID as described herein. In some embodiments, functional element 199 comprises at least one sensor, sensor 199a shown. In some embodiments, functional element 599 comprises at least one sensor, sensor 599a shown.

Functional elements 99, 199, and/or 599 can comprise one, two or more sensors configured to collect diagnostic data DD of a patient, and/or image data ID of a patient.

Functional elements 99, 199, and/or 599 can comprise at least one sensor (e.g. sensor 199a and/or 599a) that is configured to produce a signal related to tissue being captured in a coring element 155. In some embodiments, lack of detection of tissue being captured in a coring element 155 results in system 10 automatically adjusting one or more microcoring parameters (e.g. depth of penetration of element 155, velocity of element 155 advancement and/or retraction, and/or acceleration of element 155 advancement or retraction). In some embodiments, detection of tissue being captured in a coring element 155 is used to determine (e.g. automatically determine) a minimum depth of penetration of element 155. In some embodiments, functional elements 99, 199, and/or 599 comprise at least two sensors, and/or at least three sensors configured to produce a signal related to tissue being captured in a coring element 155. In the embodiments, system 10 can be configured to determine that tissue has been captured if the signals from two or three sensors each represent tissue capture (e.g. the multiple signals agree). In other embodiments, system 10 can be configured to determine that tissue has been captured if any one or more of the signals from the two or three sensors represent tissue capture. In some embodiments, functional elements 99, 199, and/or 599 comprise one, two, or more optical sensors, such as optical sensors configured to detect tissue capture and/or to determine the position of a system 10 component (e.g. the position of a movable system 10 component).

Functional element 99, 199, and/or 599 can comprise a wireless element, such as a wireless transmitter that can send and/or receive power and/or data wirelessly. In some embodiments, a functional element 99, 199, and/or 599 comprises a sensor and/or a transducer that receives power wirelessly, and/or transmits signals (e.g. recorded sensor signals) wirelessly.

Functional element 99, 199, and/or 599 can comprise one or more sensors selected from the group consisting of: accelerometer; gravity-based sensor; strain gauge; acoustic sensor (e.g. a microphone or other acoustic sensor); electromagnetic sensor (e.g. a hall effect sensor); pressure sensor; vibration sensor; temperature sensor; vacuum sensor; GPS sensor; pH sensor; optical sensor; and combinations of these.

Functional elements 99, 199, and/or 599 can comprise a patient “physiologic sensor” comprising one, two or more sensors configured to measure a patient physiologic parameter such as: body temperature; heart rate; blood pressure; respiration rate; perspiration rate; blood gas level; blood glucose level; brain and/or other neural activity such as measured by electroencephalogram (EEG), local field potential (LFP), and/or neuronal firing (e.g. single neuron firing activity); eye motion; EKG; and combinations of these.

Functional elements 99, 199, and/or 599 can comprise a patient “environment sensor” comprising one, two or more sensors configured to measure a patient “environment parameter” such as: room temperature; room humidity; room pressure; room light level; room ambient noise level; room barometric pressure; and combinations of these.

In some embodiments, functional elements 99, 199, and/or 599 comprise one or more sensors configured to measure a system 10 parameter, such as a system parameter selected from the group consisting of: temperature of at least a portion of a system 10 component; pressure and/or strain of a system 10 component; speed and/or acceleration of a system 10 component (e.g. speed and/or acceleration of a coring element 155 and/or other portion of treatment device 100); position and/or geometry of a system 10 component (e.g. position and/or geometry of a coring element 155 and/or other portion of treatment device 100); energy level; power level; and combinations of these.

In some embodiments, system 10 is configured to operate in a closed loop mode, in which one or more parameters of treatment device 100 are adjusted based on one or more recorded parameters, such as system parameters, patient physiologic parameters, and/or patient environment parameters, each as described herein. For example, algorithm 525 can analyze (e.g. continuously and/or intermittently analyze) one or more signals provided by a functional element 99, 199, and/or 599, and adjust the treatment performed by system 10 based on the analysis.

In some embodiments, functional elements 99, 199, and/or 599 comprise one or more transducers selected from the group consisting of: cooling element such as a Peltier element; heating element such as a Peltier element or a heat pump; vibrational transducer; light-producing element; a magnetic field-generating element; vacuum-generating element; and combinations of these.

In some embodiments, functional elements 99, 199, and/or 599 comprise an assembly or other component configured to provide a vacuum to another component of system 10. For example, functional elements 99, 199, and/or 599 can comprise a tissue-engaging port configured to receive a vacuum (e.g. from console 500) and to stabilize tissue, capture tissue (e.g. draw tissue toward the port) and/or otherwise engage tissue, when the vacuum is applied to the port. Functional elements 99, 199, and/or 599 can comprise a source of vacuum, such as vacuum that can be applied to such a tissue-engaging port.

In some embodiments, functional elements 99, 199, and/or 599 comprise an adhesive, and/or an adhesive dispensing component, such as when an adhesive is used to temporarily (e.g. less than 1 day) and/or chronically (e.g. at least 1 week, 1 month, or 3 months) attach a component of system 10 to tissue of the patient, and/or to another component of system 10.

In some embodiments, functional elements 99, 199, and/or 599 comprise a cooling fluid or cooling component (e.g. a thermoelectric cooling element) and/or an assembly configured to provide cooling (e.g. provide cooling to a system 10 component). In some embodiments, system 10 is configured to provide cooling to tissue and/or to a system 10 component during delivery of a tissue treatment and/or diagnosis, such as to avoid damage to non-target tissue and/or to avoid degradation of a system 10 component. Alternatively or additionally, system 10 can comprise a functional element comprising an assembly configured to provide a cooling fluid (e.g. in a recirculating arrangement) to another system 10 component.

In some embodiments, functional elements 99, 199, and/or 599 comprise an assembly or other component configured to apply a force to tissue (e.g. a grasping component configured to place tissue in tension, and/or a pushing element configured to provide a compressive force to tissue), such as to apply a force (e.g. a tensioning and/or compressing force) to tissue (e.g. target tissue) while a microcoring procedure is being performed on target tissue by another component of system 10.

Functional element 99, 199, and/or 599 can comprise an assembly configured to deliver agent 60 to the patient, as described herein. In some embodiments, agent 60 is delivered to the patient via one or more coring elements 155, where functional element 99, 199, and/or 599 comprises a pump or other fluid propulsion assembly that propels agent 60 through one or more conduits (e.g. fluid delivery tubes) such that agent 60 can be delivered into the patient (e.g. into the dermis of the patient) by one or more (e.g. all) coring elements 155 during a microcoring or other procedure performed via injection of elements 155 into the patient.

Functional element 99 can comprise a cell phone, laptop, tablet, camera, and/or other operator-maintained device. In some embodiments, data collected during a treatment procedure performed by system 10 is provided by, stored, and/or analyzed by one of these devices.

Functional element 99 can comprise a patient diagnostic device, such as a device configured to gather patient data PD (e.g. diagnostic data DD and/or image data ID).

Treatment device 100 comprises various components such as conduits 101, nozzles 102, cable 103, and housing 110. These components can be of similar construction and arrangement to the similar components described in applicant's co-pending U.S. patent application Ser. No. 17/291,235, titled “Systems and Methods for Skin Treatment”, May 4, 2021. In some embodiments, one or more assemblies and/or subassemblies of components of treatment device 100 does not include any adhesive, such as by avoiding any adhesive-enabled connection of components, for example when housing 110 comprises a multi-part construction which is assembled without the use of adhesive (e.g. connection of multiple components is accomplished through the use of snap-fits, threads, friction fits, magnetic attachment, and/or welding).

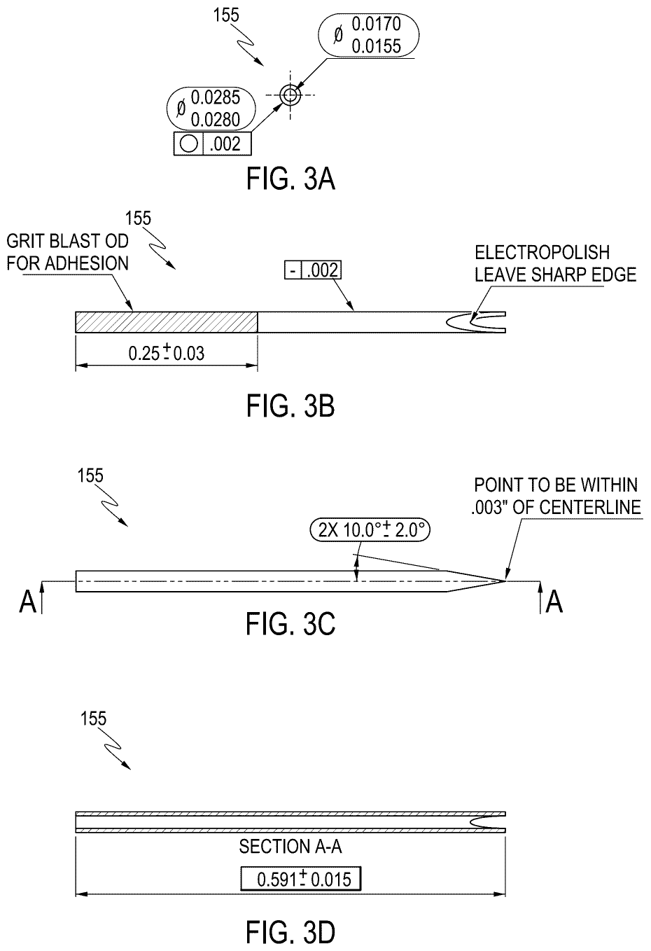

Coring elements 155 can comprise one, two or more hollow filaments, such as coring element 155 described herein in reference to FIGS. 3A-D. Each coring element 155 can comprise an elongate shaft (e.g. a hollow shaft), shaft 1551 shown, which can include a distal end. Each coring element 155 can comprise one or more projections, prong 1552 shown, that extend from the distal end of shaft 1551.

Spacer assembly 180 can comprise a housing and other components that are configured to properly position treatment module 150 relative to the patient's skin being treated. Spacer assembly 180 can include one or more sensors, sensor 181 shown, which can be configured to detect proper engagement of spacer assembly 180 with the patient (e.g. proper pressure level detected).

Actuation assembly 120 can be configured to interface with treatment module 150 by performing a function selected from the group consisting of: control the motion of a treatment module 150 (e.g. translate treatment module 150 along one, two, or three axes); activate one or more components of treatment module 150 (e.g. advance and/or retract one or more coring elements 155 into and/or from tissue); rotate one or more components of treatment module 150 (e.g. rotate one or more coring elements 155 prior to, during, and/or after their insertion into tissue); vibrate one or more components of treatment module 150; and combinations of these. Actuation assembly 120 comprises actuator 121 shown. Actuator 121 and other components of actuation assembly 120 can be of similar construction and arrangement as the similar components described in applicant's co-pending U.S. patent application Ser. No. 17/291,235, titled “Systems and Methods for Skin Treatment”, May 4, 2021.

Console 500 can comprise user interface 510 as shown, which can comprise one or more user input and/or user output components, such as one, two or more components selected from the group consisting of: display; touch screen display; button; switch; foot switch; lever; membrane keypad; mouse, joystick; microphone; speaker; vibrational and/or other haptic transducer; light such as a light emitting diode; and combinations of these. Console 500 can comprise controller 520 as shown, which can include: one or more central processing units (CPUs), microprocessors and/or other microcontrollers, processor 521 shown; a memory storage component, memory 522 shown (e.g. volatile or non-volatile memory); one or more sets of instructions, instructions 523 shown; signal processing and other electronic circuitry; oscillator circuitry such as voltage-controlled oscillator (VCO) circuitry; analog to digital circuitry; digital to analog circuitry; and/or other componentry configured to control or otherwise interface with one or more components of system 10. Controller 520 can comprise a power supply and/or energy storage component (e.g. a battery, a capacitor, and/or a power supply converted to receive “wall power” and convert it to an AC or DC voltage for use by system 10). Console 500 can further comprise drive module 550, and vacuum assembly 560, each as shown. Console 500 and its various components can be of similar construction and arrangement to those described in applicant's co-pending U.S. patent application Ser. No. 17/291,235, titled “Systems and Methods for Skin Treatment”, May 4, 2021.

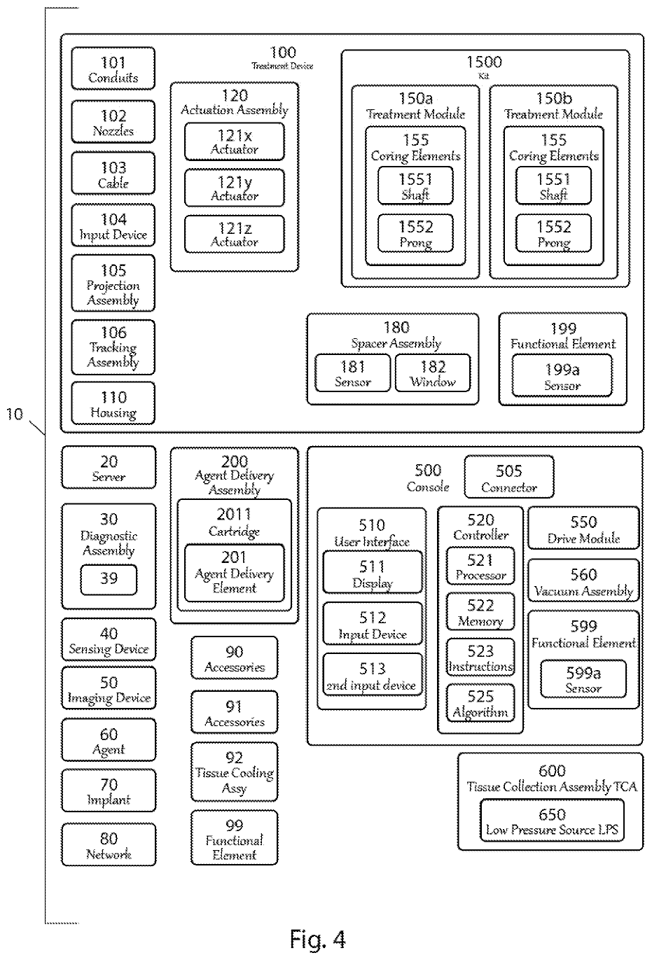

System 10 can include one or more accessory components, accessories 90 shown. Accessories 90 can include one or more accessory components, such as those described in reference to FIG. 4 herein.

As used herein, a “treatment plan” comprises a set of parameters that are used in treating target tissue of the patient using system 10. A treatment plan can include a set of treatment settings, such as one, two or more microcoring parameters. A treatment plan can include a set of different medical procedures (e.g. one, two or more microcoring procedures and/or other treatment procedures). A treatment plan can include a desired and/or recommended order for performing a set of multiple medical procedures (e.g. where the treatment plan provides multiple procedures to be performed in a particular order, where in some instances sufficient efficacy is achieved when a subset of the procedures is performed). In some embodiments, system 10 is configured to automatically and/or semi-automatically (“automatically” herein) generate a treatment plan (e.g. one or more treatment plans made available to a clinician). System 10 can generate a treatment plan using an algorithm, such as algorithm 525 described herein. A treatment plan can be developed by algorithm 525 using at least image data ID, such as by using image data ID comprising: ultrasound-based image data (e.g. Doppler data and/or other image data produced using ultrasound); CT-based image data; MRI-based image data; and/or X-ray-based image data (e.g. fluoroscopic data and/or other image data produced using X-ray). Alternatively or additionally, algorithm 525 can develop a proposed treatment plan based on parameters selected from the group consisting of: patient age; patient race; patient gender; patient skin type; patient skin condition; volume of target tissue to be treated; cellulite and/or fat content of target tissue; geometry of target tissue; tissue type, geometry and/or other characteristic of non-target tissue proximate the target tissue; and combinations of these. In some embodiments, a treatment plan includes a methodology to ensure treatment of target tissue, while avoiding damage to neighboring non-target tissue. In some embodiments, system 10 (e.g. via algorithm 525) is configured to produce a prediction of outcome (e.g. an estimation of likelihood of efficacy and/or an assessment of any risks) associated with one or more treatment plans.

System 10 can comprise algorithm 525 shown, which can comprise one or more algorithms. Algorithm 525 can comprise one or more algorithms that are performed by processor 521 of controller 520. Processor 521 can perform algorithm 525 using instructions 523, such as instructions 523 that are stored in memory 522 of controller 520. All or a portion of algorithm 525 can be integrated into one, two or more of various components of system 10, such as console 500 (as shown), treatment device 100, imaging device 50, TCA 600, and/or functional element 99. Algorithm 525 can comprise one or more machine learning, neural network, and/or other artificial intelligence algorithms (“AI algorithm” herein).

Algorithm 525 (e.g. an AI algorithm) can be configured to determine and/or modify one or more microcoring parameters, such as to effectively treat target tissue (e.g. improve cosmesis of the patient) and/or avoid damage to non-target tissue. For example, algorithm 525 can be configured to determine a volume of target tissue to be treated (e.g. treated with a microcoring procedure), such as to effectively enhance cosmesis of the patient and/or otherwise provide a therapeutic benefit to the patient, while avoiding or at least minimizing damage to non-target tissue. In these embodiments, algorithm 525 can be further configured to determine and/or modify one or more microcoring parameters (e.g. at least based on the determined volume), such as to effectively treat the target tissue volume determined, while avoiding damage to non-target tissue, as described hereinabove.

Algorithm 525 can be configured to perform a “microcoring analysis” comprising using an analysis of one or more types of information by algorithm 525 to assess the level of microcoring (e.g. the current level of microcoring) of target tissue. The results of this analysis can be used by system 10 to perform microcoring in a closed loop mode. Microcoring data produced in the microcoring analysis can be stored as image data ID (e.g. and correlated with one or more tissue locations). In some embodiments, system 10 (e.g. treatment device 100 and/or imaging device 50) delivers and/or receives energy (e.g. light energy and/or ultrasound energy or other imaging-capable energy) to and/or from tissue, and algorithm 525 performs a microcoring analysis based on the delivered and/or received energy.

Algorithm 525 can be configured to adjust tissue treatment parameters (e.g. microcoring parameters) based on sensor signals, such as when sensor 199a provides feedback to algorithm 525 regarding a microcoring procedure.

In some embodiments, algorithm 525 is configured to perform an analysis on patient data PD (e.g. patient use data from a single patient, or a group of patients upon which system 10 has performed a treatment procedure), such as to modify a future treatment provided by system 10.

In some embodiments, algorithm 525 is configured to provide a treatment plan, such as when algorithm 525 performs analysis on patient data PD comprising data collected during treatment of the patient with system 10 in a previous treatment procedure, and/or based on patient data PD collected from use of system 10 on multiple patients (e.g. a large number of patients treated with system 10).

System 10 can include network 80 as shown, which can comprise one or more computer networks such as the Internet, a local area network, cellular network, and/or other data sharing, storage, and/or transmitting platform. In some embodiments, patient data PD, and/or other data collected during the use of system 10 is transmitted from one location to another location over network 80. In some embodiments, one or more central data storage areas are used to store the data, such as when an algorithm 525 analyzes the data to provide a treatment plan and/or provide system 10 parameters for a future treatment of one or more patients.

Treatment device 100 and/or another component of system 10 can be configured to perform a treatment (e.g. a microcoring treatment) in a closed loop mode (i.e. a closed loop mode of microcoring and/or other closed loop mode of operation), such as when one or more sensors of system 10 (e.g. a sensor-based functional element 99, 199, and/or 599), provide patient and/or system information that is used to continuously and/or intermittently adjust the treatment being delivered by treatment device 100 (e.g. adjust the microcoring parameters and/or other parameters of the treatment). For example, microcoring can be adjusted in a closed loop mode based on a system 10 parameter and/or based on a patient parameter (e.g. a patient physiologic parameter, patient anatomical parameter, and/or a patient environment parameter, each as described herein). Microcoring by treatment device 100 can be adjusted based on image data ID described herein, such as to redirect and/or otherwise adjust microcoring (e.g. due to detected patient motion and/or undesired treatment device 100 motion) and/or to change one or more microcoring parameters (e.g. as determined by algorithm 525 using image data ID or other data). In some embodiments, image data ID is used to determine when a treatment (e.g. a microcoring amount) is sufficient, such as when algorithm 525 analyzes image data ID to confirm sufficient change in tissue characteristics have occurred.

As described herein, system 10 can be configured to perform a series of clinical procedures on a patient, such as a patient desiring improved cosmesis of the face or other body location, as described herein. In some embodiments, system 10 is configured to be used to: perform a first procedure and a second procedure, in which the two procedures are performed at least 24 hours apart. The first procedure can include microcoring, the second procedure can include microcoring, or both can include microcoring. In some embodiments, the first procedure does not include microcoring, while the second procedure does include microcoring. In some embodiments, two, three, four, or more microcoring procedures of the present inventive concepts are performed, such as over a period of months and/or years. In some embodiments, the treatment plan for a subsequent procedure using system 10 is based on the data collected and/or results of one or more previous treatment procedures performed using system 10.

System 10 can be configured to perform a treatment on a patient (e.g. a patient desiring improved cosmesis of the face or other body location) that includes the performance of multiple, sequential treatment plans, such as a sequence of treatment plans that each may use one, two or more components of system 10 (e.g. one, two or more of treatment devices 100) that are used to perform one or more diagnostic procedures, and/or one or more therapeutic procedures. Performance of an “initial treatment plan” performed using system 10, can be configured based on current physiologic state (e.g. current undesired state of tissue) of the patient, as well as any previous treatments performed (e.g. using system 10 or otherwise). Each “subsequent treatment plan”, can also be based on the current physiologic state, as well as all previous treatments performed, as described herein.

In some embodiments, the one or more coring elements 155 (e.g. three coring elements 155) comprise a dimension selected from the group consisting of: an outer diameter of no more than 0.050 in, or no more than 0.040 in, such as approximately 0.028 in; an inner diameter of no more than 0.030 in, or no more than 0.025 in, such as approximately 0.016 in; a core length of at least 0.5 mm and/or no more than 5.0 mm; a penetration depth of no more than 6.0 mm; a cutting depth of no more than 5.0 mm; and combinations of these.

In some embodiments, one or more coring elements 155 comprise a double-beveled needle geometry (e.g. as shown in FIGS. 3A-D), such as to minimize effective insertion depth and/or resist wear during use.

In some embodiments, system 10 is configured to precisely control insertion speed of the one or more coring elements 155 (e.g. simultaneous insertion of all of coring elements 155). In these embodiments, the dwell time can comprise a time of no more than 60 msec, such as no more than 45 msec, no more than 30 msec, and/or no more than 20 msec. System 10 (e.g. console 500 and/or treatment device 100) can comprise a proportional integral derivative (PID) controller that provides closed loop control of coring element 155 advancement and position that results in accurate core depth, such as while minimizing impact forces on the patient's skin (e.g, thus improving healing response and core hole precision).

In some embodiments, multiple coring elements 155 are positioned in an array (e.g. a linear arrangement of three or four elements 155) in which the coring elements 155 are separated by a distance of at least 0.2 mm, such as at least 0.5 mm, at least 1.0 mm, at least 2.0 mm, and/or approximately 3.33 mm.

System 10 can include tissue collection assembly 600 for clearing tissue cores captured by coring elements 155. In some embodiments, LPS 650 comprises a single source of low pressure (e.g. vacuum) that provides multiple (e.g. two) functions. System 10 can be configured to control the flow rate (e.g. the pressure) proximate the coring elements 155, such as to remove tissue cores without impacting low pressure applied to spacer assembly 180 (e.g. spacer assembly 180 using suction to stabilize treatment module 150 relative to the patient's skin). The flow channels into which the tissue cores are extracted can include a funnel portion that increases the flow velocity at locations where the tissue is extracted from the back ends of the coring elements 155.

Treatment device 100 can comprise spacer assembly 180, which can provide a stabilizing force to treatment device 100 during use, as described herein. For example, spacer assembly 180 can utilize a suction force that allows effective treatment of target tissue areas comprising various surface contours. System 10 can include an automated pinch valve in line with vacuum conduits provided to spacer assembly 180, such as to provide enhanced stabilization of treatment module 150 with the patient's skin between patterns of deployment of one or more coring elements 155. For example, the pinch valve can be activated to allow easy repositioning of treatment module 150 (e.g. and spacer assembly 180) at the end of a pattern of microcoring, such as to improve ease and speed of a treatment.

Treatment device 100 can comprise a “treatment window” that is sized to accommodate various ranges of suction force to be applied. In some embodiments, spacer assembly 180 provides a treatment window of at least 100 mm², such as no more than 2,000 mm², such as approximately 640 mm², such as to provide a nominal holding force of treatment module 150 (e.g. spacer assembly 180) of at least 10.0 N, such as at least 18.0 N, such as approximately 28.5 N with the patient's skin.

System 10 can be configured to detect (e.g. and quantify) deceleration of coring elements 155, such as to minimize damage to the coring elements 155 and/or to detect damage to at least one coring element 155.

System 10 can include various features that enhance positioning accuracy (e.g. during deployment) of coring elements 155, such as positioning accuracy in X and Y directions, and/or positioning accuracy in the Z dimension (e.g. insertion direction). Such features include but are not limited to: 1:1 gearing and/or direct drive in actuation assembly 120; sensor detection of position (e.g. hall sensors and/or optical sensors such as optical encoders); linear bearings (e.g. that minimize undesired motion and/or creep from a desired position); and combinations of these.

System 10 can be configured to provide variable patterns for microcoring (e.g. varied microcoring density), such as to achieve a skin removal percentage (also referred to as “areal fraction”) of no more than 20%, and/or no less than 0.5%, such as at least 1%, and/or at most 10% (e.g. between 1% and 10%).

Actuation assembly 120 can comprise one or more actuators (e.g. solenoids) that are configured to precisely control movement of one or more coring elements 155 such as to achieve variable depth control within 0.8 mm, such as within 0.5 mm, while accommodating variability in skin thickness, skin toughness, and/or other varying skin parameters.

System 10 can comprise a calibration routine such as to store calibration information created during manufacturing of one or more components of system 10, and/or information collected at a clinical site (e.g. prior to, during, and/or after use of system 10). Calibration data can be stored in a treatment module 150, actuation assembly 120, and/or other component of treatment device 100. System 10 can be configured to improve accuracy of needle deployment (e.g. in the Z direction), based on the calibration data (e.g. to accommodate variability in manufacturing processes). In some embodiments, system 10 includes a calibration device (e.g. accessory 90 comprises a calibration device). The calibration device can comprise a device (e.g. an electromechanical device) that includes one or more sensors (e.g. functional element 99 comprising one or more sensors) that are configured to detect the position of actuation assembly 120 (e.g. provide signals representing the position of one or more movable portions of assembly 120 relative to treatment device 100). The detected position values can be used by an algorithm of system 10 (e.g. algorithm 525) to calibrate actuation assembly 120 (e.g. assist in a manual calibration of actuation assembly 120 and/or automatically calibrate actuation assembly 120). In some embodiments, accessory 90 comprises a calibration device that includes a functional element 99 that comprises a sensor configured to produce a signal related to the position of actuation assembly 120. The sensor-based functional element 99 can comprise one, two, or more sensors selected from the group consisting of: an optical sensor; a magnetic sensor (e.g. a magnetic sensor that detects the position of one or more magnetic portions of actuation assembly 120); a force sensor; a sound sensor such as an ultrasound sensor; a density sensor; and combinations of these. In some embodiments, accessory 90 comprises a calibration device that includes a functional element 99 that comprises at least two of: an optical sensor; a magnetic sensor (e.g. a magnetic sensor that detects the position of one or more magnetic portions of actuation assembly 120); a force sensor; a sound sensor such as an ultrasound sensor; and/or a density sensor, where each sensor is configured to produce a signal related to the position of actuation assembly 120.

Coring elements 155 can comprise a bevel angle of no more than 30 degrees, such as no more than 25 degrees, and/or no more than 20 degrees, such as to improve healing and/or minimize scarring of the patient.

System 10 can be configured to control the speed and/or frequency (e.g. repetition rate) of the deployment of the coring elements 155 into the patient's skin, such as to deploy the elements 155 (e.g. three elements 155 in unison) at a rate of at least 1 Hz, or 3 Hz, or approximately 8 Hz. Alternatively or additionally, system 10 can be configured to deploy the elements 155 (e.g. three elements 155 in unison) at a rate of no more than 30 Hz, such as no more than 20 Hz, such as approximately 8 Hz.

In some embodiments, one or more components of system 10 comprise a modular arrangement of components, for example an arrangement where one or more assemblies of system 10 can be included (e.g. provided to a user of system 10) in a redundant fashion (e.g. provided as a pair or other multiple of the same component), such that each assembly can be easily replaced if damaged or otherwise is malfunctioning. For example, one or more assemblies of console 500, such as one or more control boards, a vacuum pump, and/or the display of user interface 510 can be provided in redundant fashion and configured to be easily removed (e.g. with the use of no or minimal tools) such as to be easily replaceable. Additionally or alternatively, the cable connecting treatment device 100 to console 500 can be provided in redundant fashion and include simple connectors, such as to be easily removable and/or replaceable (e.g. when the cable comprises dual-end connectors).

In some embodiments, system 10 includes a tissue removal tool, for example when accessory 90 comprises a tissue removal tool. The accessory 90 comprising the tissue removal tool can be configured to remove tissue cores after treatment device 100 punctures tissue to create one or more cores. For example, some cores created by treatment device 100 may not be fully removed by coring element 155, and these cores can be subsequently removed by the tissue removal tool. The tissue removal tool can comprise a cloth (e.g. to wipe remaining cores), an insertable filament, and/or a vacuum-based tool configured to remove remaining cores with suction.

Applicant has conducted various studies using the systems, devices, methods, and other technologies of the present inventive concepts, such as system 10 and its components as described herein. Applicant has conducted studies using the systems of the present inventive concepts in mammalian subjects, including multiple studies in porcine models as well as human patients. Results of these studies are described in applicant's co-pending International PCT Patent Application Serial Number PCT/US2022/030236, titled “Skin Treatment Systems and Methods”, filed May 20, 2022.

System 10 can be configured to remove skin via microcoring, such as without use of thermal energy (e.g. avoiding damage to cells from heating) during the microcoring procedure. Energy-based devices such as fractional laser and radiofrequency ablation lead to epidermal and dermal cell necrosis from thermal injury that may inhibit rapid wound closure, an adverse effect that can be avoided via use of system 10. Although fractional lasers and radiofrequency devices have shown acceptable results in rejuvenation of skin, data on skin tightening is inconclusive. It is suspected that coagulation necrosis of the cells surrounding fractional laser cores prevent early wound closure and therefore limit reduction of skin surface area and skin tightening. System 10 avoids coagulation necrosis and can achieve both early wound closure, and enhanced skin tightening, as described herein. The coring elements 155 and other components of system 10 provide numerous benefits including limited side effects, and fast (e.g. expedited) patient recovery. By removing tissue, significant skin tightening can be achieved, as demonstrated by data gained in clinical procedures performed on human patients.

System 10 can be configured to both tighten skin and reduce skin wrinkles and/or folds of the patient's skin. Use of system 10 in human patients has achieved skin tightening as well as reduction in skin wrinkles and/or folds, via removal of skin without the use of thermal energy, while also reducing (e.g. preventing or resulting in minimal) scar formation.

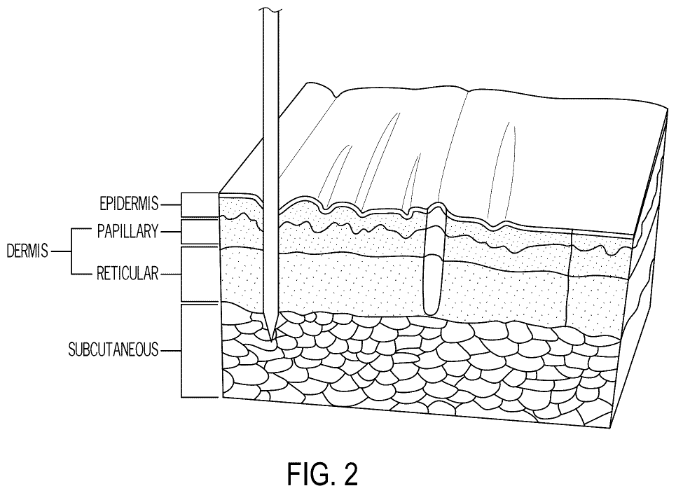

FIG. 2 illustrates a coring element 155 being safely introduced into the skin, such as to subsequently be withdrawn to remove a microcore of tissue, such that the remaining tissue heals with no scarring or at most minimal scarring. The treatment provided by system 10 also provides near-immediate closure along the relaxed skin tension lines (RSTLs), with no thermal energy.

Referring now to FIGS. 3A-D, various views of a coring element are illustrated, consistent with the present inventive concepts. Typical dimensions of a coring element 155 are shown. In some embodiments, coring element 155 comprises a penetrating portion with an outer diameter of at least 0.0203″ and/or an outer diameter of no more than 0.0500″. In some embodiments, coring element 155 comprises a penetrating portion with an inner diameter of at least 0.0103″ and/or an inner diameter of no more than 0.0207″.

Referring now to FIG. 4, a block diagram of a tissue treatment system is illustrated, consistent with the present inventive concepts. As shown, system 10 of FIG. 4 includes treatment device 100, console 500, tissue collection assembly 600, and other components as shown, each of which can be of similar construction and arrangement to the similar components described in reference to system 10 of FIG. 1 described herein.

Treatment device 100 can be configured as a handheld device, comprising a “handpiece” geometry.

Console 500 can comprise user interface 510 as shown. In some embodiments, at least a portion of user interface 510 is integral to treatment device 100. User interface 510 can be configured to allow a user (e.g. a clinician) to set one or more microcoring parameters, such as depth of penetration of coring elements 155, density of coring (e.g. density of coring created by an array of one, two, three or more elements 155 of treatment module 150, and/or other coring and/or system 10 parameters. In some embodiments, system 10, via user interface 510, is configured to provide an automated presentation of: pre-treatment setup steps of system 10; intra-treatment use of system 10; and/or post-treatment steps of system 10.

Treatment device 100 can include one or more treatment modules 150, which can include a single coring element 155 or multiple coring elements 155 (e.g. three coring elements 155). In some embodiments, treatment device 100 includes a kit 1500 of multiple treatment modules 150, such as a kit including at least one treatment module 150a each with a single coring element 155, and at least one treatment module 150b each with multiple (e.g. three) coring elements 155. For example, a treatment module 150a with a single coring element 155 can be used to perform microcoring in one or more “hard to reach areas”, while treatment module 150b can be used to perform microcoring in skin surface areas that are larger (e.g. to reduce treatment time than that achievable via a treatment module 150a with a single coring element 155). Treatment module 150b can comprise an assembly of multiple (e.g. three) coring elements 155 that are positioned at least 1 mm, at least 2 mm, at least 3 mm, and/or approximately 3.33 mm apart (e.g. to reduce likelihood of skin “tenting” and/or to reduce slicing of skin). In some embodiments, treatment module 150b can comprise an assembly of multiple (e.g. three) coring elements 155 that are positioned no more than 7 mm apart, such as no more than 6 mm apart, no more than 5 mm apart, and/or no more than 4 mm apart.

In some embodiments, treatment module 150b comprises an assembly of multiple (e.g. three) coring elements 155, where each element 155 comprises opposing lateral sides (e.g. lateral sides 10-8231 and 10-8233 as shown in FIG. 32) that terminate at the distal end of each element 155 in one or more sharpened edges that each define a cutting axis (e.g. sharpened edges of pairs of prongs 10-8121 that define axes A1, A2, and A3 shown in FIG. 20). In some embodiments, the cutting edges axes of the cutting edges of multiple (e.g. 3) coring elements 155 are arranged in a non-linear arrangement (e.g. the axes are positioned in a parallel, as shown in FIG. 20, or angular-offset arrangement), such as to prevent slicing and/or tearing of skin positioned between coring elements 155 during microcoring (e.g. slicing and/or tearing that might result from multiple linearly aligned cutting edges of relatively close proximity being inserted through the skin simultaneously).

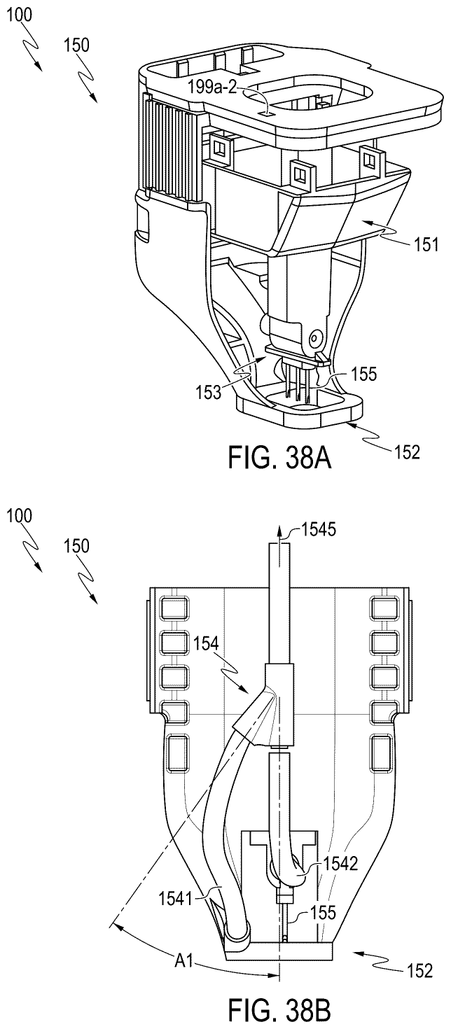

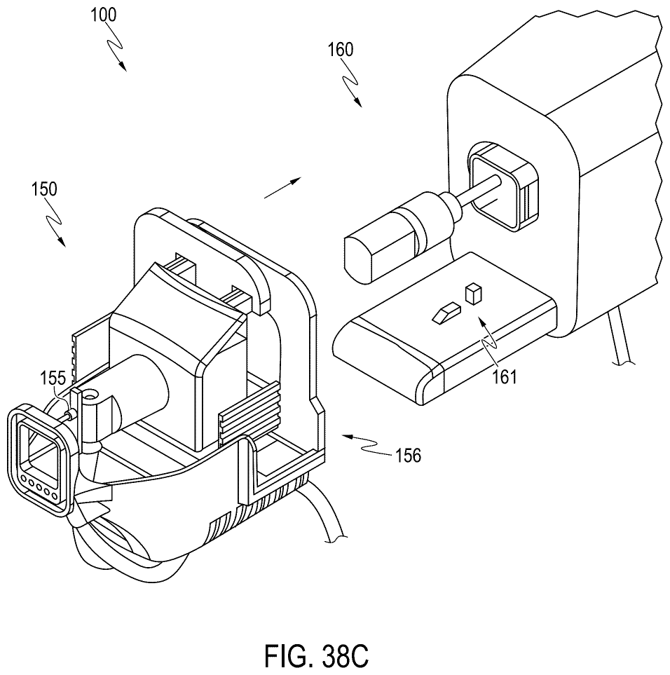

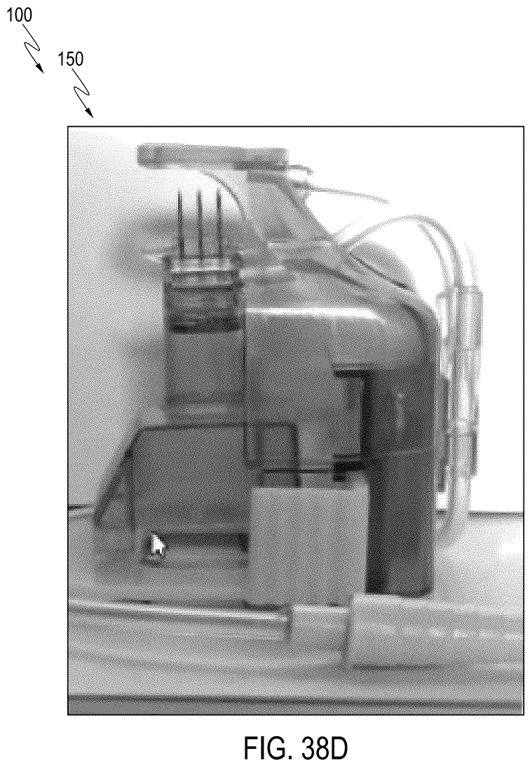

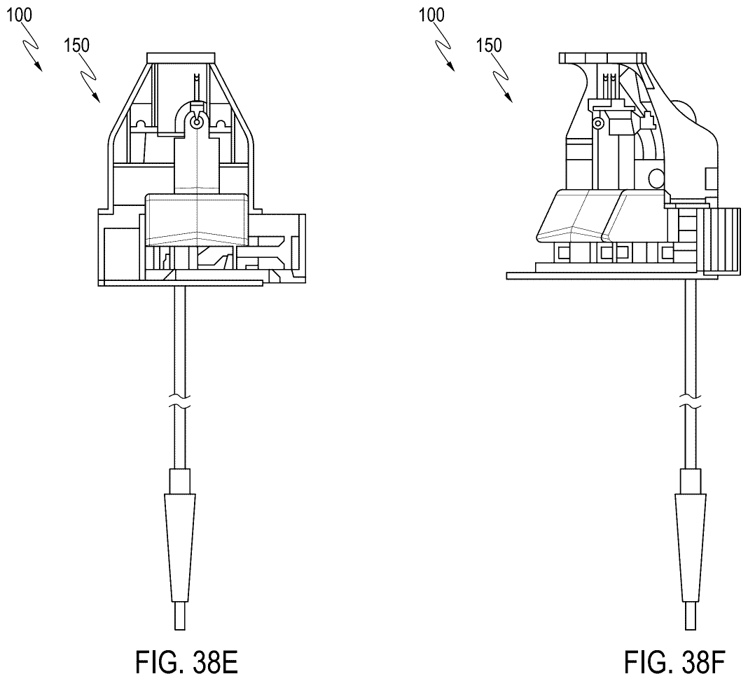

Treatment module 150 can comprise a single assembly that is attached to the remaining portion of treatment device 100, and it can include one or more mechanisms to prevent undesired movement of microcoring elements 155 when not attached, such as is described in reference to FIG. 38C herein.

Treatment device 100 can include actuation assembly 120 which can include one or more actuators, such as x-actuator 121x, y-actuator 121y, and/or z-actuator 121z as shown. In some embodiments, actuation assembly 120 includes x-actuator 121x and y-actuator 121y for positioning the coring elements 155 relative to one or more locations on the patient's skin, and z-actuator 121z is configured to advance the elements 155 into the skin, such as is described in detail hereinbelow. This x-y positioning, and z-advancement can be repeated multiple times until a desired microcoring pattern is achieved. Actuators 121 (e.g. x-actuator 121x and y-actuator 121y) can comprise motors, such as brushless DC motors, as well as a fine-pitched lead screw (e.g. a lead screw with a screw comprising a M3 0.5-6 g thread or similar). The lead screw can comprise a brass or other metal lead screw with a polyether ether ketone (PEEK) or other plastic projection that rides on the screw threads. Actuators 121 (e.g. x-actuator 121x and y-actuator 121y) can comprise a motor with at least a 3:1 or 4:1 gear ratio. Actuators 121 can comprise one or more position sensors (e.g. functional elements 199a configured as position sensors), such as hall effect sensors. In some embodiments, x-actuator 121x and/or y-actuator 121y include an optical gate-based position sensor. In some embodiments, z-actuator 121z comprises a position sensor (e.g. a functional element 199a configured as a position sensor), such as a linear magnetic and/or optical encoder configured to determine the change in position of a translating component of actuator 121z (e.g. determine the change in position of the translating component of actuator 121z, such as to determine the acceleration, speed, and/or absolute position of advancement and/or retraction of coring elements 155 by actuator 121z). In some embodiments, z-actuator 121z comprises a position sensor that includes or is integral to a linear bearing, such as to ensure unimpeded motion of z-actuator 121z.

System 10 can be configured to perform a microcoring of tissue by the user (e.g. the clinician) positioning a skin contacting surface (e.g. a frame as described herein) of treatment device 100 at a first tissue surface location. Activation of coring (e.g. via a footswitch or other control of system 10) is initiated by the user, and the following sequence of events occur: (1) treatment module 150 (e.g. including one or more coring elements 155, such as three coring elements 155) is advanced into tissue (e.g. via z-actuator 121z to a target depth); (2) treatment module 150 is withdrawn from the tissue (e.g. via z-actuator 121z); and (3) treatment module 150 is repositioned to a new location (e.g. via x-actuator 121x and/or y-actuator 121y). Steps 1 thru Step 3 can be performed a single time, or multiple times, such as at least 3, 8, 12, 17, and/or 20 times (e.g. with a three coring element 155 assembly), in order to perform a “treatment event”. After a first treatment event is performed, one or more subsequent treatment events can be initiated. In each event, the user positions the skin contacting surface (e.g. a frame as described herein) at a desired (e.g. new) tissue location, and one or more series of Steps 1 thru 3 are repeated.

In some embodiments, a switch (e.g. a footswitch) and/or other control that is activated by a user to initiate a treatment event, must remain activated (e.g. a footswitch must continue to be depressed) in order for the treatment event to continue to completion (e.g. of the one or more series of Steps 1 thru 3 that are repeated). If the control is not maintained in an active state (e.g. a footswitch pedal is released), system 10 can be configured in the following arrangement: if Step 1 or Step 2 is in process, the treatment procedure continues thru completion of Step 2 (i.e. completion of needle withdrawal), and an additional coring element 155 advancement of Step 1 is prevented); otherwise (e.g. if Step 3 is in process), the treatment procedure stops. This configuration provides a safe mode of operation as well as allowing a user to treat a portion of a proposed treatment area of a treatment event.

System 10 can include a limit on depth of travel of actuator 121z (e.g. limit depth of travel of a translating component of actuator 121z that translates along the z-axis), such as to limit depth of penetration of one or more coring elements 155 into skin (e.g. such as to avoid contact of a coring element 155 with a nerve, blood vessel, and/or bone). In some embodiments, this depth of penetration is input by an operator (e.g. input into user interface 510), such as a depth of 3 mm, 4 mm, and/or 5 mm of penetration into the patient's skin. In some embodiments, a titration or other iterative adjustment procedure is performed by a user (e.g. a clinician), in which depth of penetration is adjusted, such as to increase depth of penetration to achieve sufficient coring, and/or a decrease in depth of penetration (e.g. when treating an area in which element 155 contact with bone might otherwise result). System 10 can be configured to decelerate actuator 121z as the one or more microcoring elements 155 are approaching a target depth.

Actuator 121z can be controlled by one or more algorithms of system 10 (e.g. algorithm 525) via one or more sets of instructions. Ranges of positions of a translating portion of actuator 121z range from a −zₘₐₓ to zₘₐₓ, with a rise time tᵣ. The translating component of actuator 121z has a velocity, v, and a maximum velocity vₘₐₓ, and an acceleration, a. The acceleration a of the translating component of actuator 121z can be controlled to approximate a smooth continuous function. The translating component of actuator 121z can have a zₘₐₓ equal to approximately 0.007 m (0.7 cm), and vₘₐₓ can be equal to approximately 1.0 m/sec. The translating component of actuator 121z can have a velocity function as follows:

$$The maximum velocity occurs at {t}_{r} = 0 {υ}_{max} = \frac{4 \cdot {z}_{max}}{{t}_{r}}$$

Integrating gives the position:

$$z = {z}_{max} \cdot ( \frac{2 \cdot t}{{t}_{r}} + \frac{1}{π} \cdot sin ( \frac{2 \cdot π \cdot t}{{t}_{r}} ) ) At t = \frac{{t}_{r}}{2} and t = \frac{- {t}_{r}}{2} z = - {z}_{max} and {z}_{max}$$

With zₘₐₓ and vₘₐₓ specified, tᵣ is:

$${t}_{r} := 4 \cdot \frac{{z}_{max}}{{υ}_{max}} = 0.028$$

Treatment device 100 can be void of any surface, projection, and/or other mechanical stop that is contacted by treatment module 150 during advancement of coring elements 155 in a microcoring procedure (e.g. no mechanical stop is used to limit advancement of coring elements 155 during a microcoring procedure). Avoidance of such a mechanical stop can provide numerous advantages, such as avoiding the vibration that occurs when a moving assembly makes contact with a mechanical stop (e.g. a vibration that can cause a degradation of tissue cores removed during a microcoring procedure).

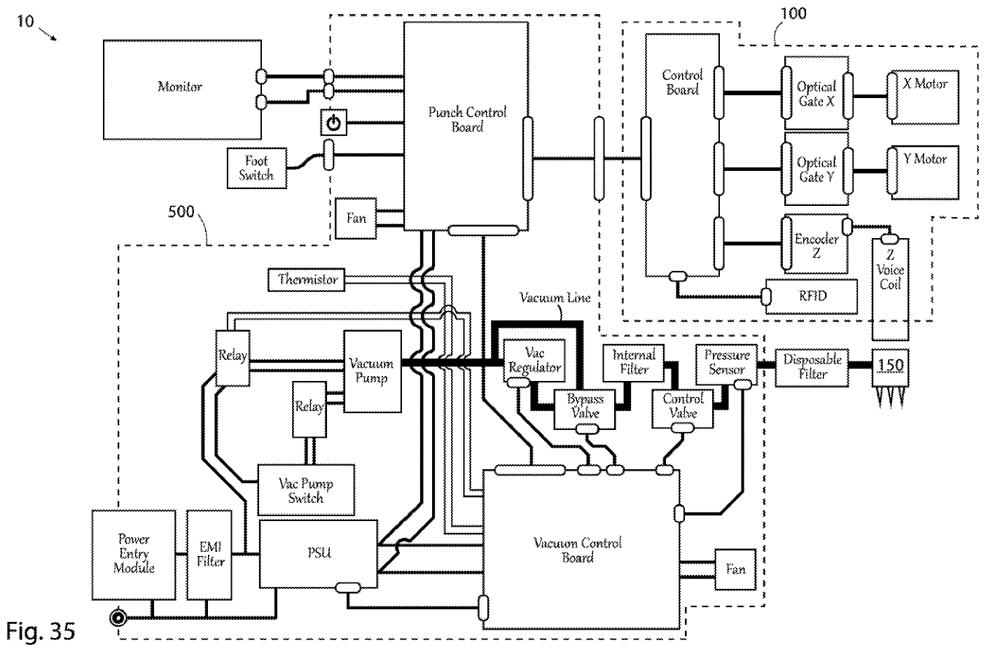

Treatment device 100 can comprise one or more sensors, such as sensor 199a shown. Console 500 can comprise one or more sensors, such as sensor 599a shown. Sensor 199a and/or 599a can comprise one or more sensors as shown in FIG. 35. Sensor 199a and/or 599a can comprise a pressure sensor, such as a pressure sensor configured to monitor a pressure level (e.g. a vacuum pressure level) of one or more conduits or other cavity portions of system 10. Sensor 199a and/or 599a can comprise one, two, or more sensors configured to monitor (e.g. constantly monitor during use) the position (e.g. the x, y, and/or z position) of actuation assembly 120 (e.g. monitor the position of an actuator 121). Sensor 199a and/or 599a can comprise one, two or more sensors configured to monitor the position of a component of system 10, such as the position of a component of an actuator 121. For example, sensor 199a and/or 599a can comprise an encoder (e.g. a magnetic and/or optical encoder), such as when comprising one, two, three, or more encoders that monitor the position of one or more components of actuator 121 (e.g. in x, y, and/or z directions). In some embodiments, the encoder comprises one or more absolute position encoders that each produce a signal related to x, y, and/or z positions of an actuator 121 (e.g. encoders configured to measure movement in all three of x, y, and z positions of actuator 121). In some embodiments, sensor 199a and/or 599a comprises a position sensor (e.g. an encoder) which monitors a position of a component with a resolution of 0.5 mm or less. For example, sensor 199a and/or 599a can comprise one or more position sensors (e.g. one or more encoders) that monitor the one or more positions of actuator 121x and/or 121y with a greater precision than 60 μm (e.g. a resolution of 60 μm or less), such as greater precision than 40 μm, such as a resolution of approximately 20 μm. Alternatively or additionally sensor 199a and/or 599a can comprise one or more position sensors (e.g. one or more encoders) that monitor one or more positions of actuator 121z with a greater precision than 5 μm (e.g. a resolution of 5 μm or less), such as a greater precision than 4 μm, 3 μm, and/or 2 μm, such as a resolution of approximately 1 μm. In some embodiments, sensor 199a and/or 599a comprises an “end of travel” switch, such as one or more switches positioned at the most proximal and/or most distal position of an actuator (e.g. an actuation assembly 120) configured to indicate when the actuator is positioned in its most proximal and/or most distal position.

In some embodiments, system 10 is configured to constantly (e.g. always) determine and/or otherwise know the position of treatment module 150 and/or its coring elements 155 (e.g. constantly determine and/or otherwise know the x, y, and/or z positions of each element 155 during use). Alternatively or additionally, sensor 199a and/or 599a can comprise one, two, or more sensors configured to monitor (e.g. constantly monitor during use) the speed and/or the acceleration of actuation assembly 120 (e.g. monitor the speed and/or acceleration of an actuator 121). System 10 can comprise one or more position, speed, and/or acceleration limits (e.g. threshold above or below which operation is prevented or at least requires additional attention from an operator). System 10 can include a set of position, speed, and/or acceleration thresholds, such as for any actuator 121. Detection of any of these parameters outside of an expected range can result in system 10 entering a warning state, such as a state in which further operation is limited (e.g. microcoring is prevented) until further action is taken. For example, system 10 can be configured to enter an alert state (e.g. alarm or otherwise prevent further operation and/or alert an operator with an audible, visual, and/or tactile alarm) if one of the following conditions are detected (e.g. as determined by signals provided by sensor 199a and/or 599a): intended depth of penetration of a microcoring element 155 not achieved; velocity profile of a microcoring element 155 motion is outside of an intended window (e.g. element 155 does not reach an intended location within a time window); a microcoring element 155 at an undesired position; acceleration of a microcoring element 155 exceeds a threshold (e.g. is above a threshold); and combinations of these. Sensor 199a and/or 599a can comprise one or more sensors configured to monitor current, such as current applied to an actuator 121 (e.g. x-actuator 121x, y-actuator 121y, and/or z-actuator 121z). System 10 can be configured to monitor current value of current, peak current, and/or amount of current over time. Current exceeding a threshold (e.g. is above a threshold) can correlate to an actuator 121 having to exert an undesired amount of force, and/or an actuator 121 being in a “stuck” position. Sensor 199a and/or 599a can comprise one or more sensors configured to detect a “locked” or “unlocked” status of treatment module 150, such as to prevent use (e.g. prevent microcoring) if treatment module 150 is not properly positioned in treatment device 100.

System 10 can be configured to monitor repeated use of treatment module 150, such as when an upper limit of uses is applied by system 10. For example, each treatment module 150 can comprise a unique identifier (e.g. an RFID or other identifier as described herein), and system 10 can keep track of uses of treatment module 150 such as to prevent repeated use above a threshold.

In some embodiments, system 10 is configured to monitor deceleration of actuator 121z, such as, to reduce the likelihood of damage to one or more coring elements 155. Sensor 199a and/or 599a can comprise one or more sensors to detect a deceleration “fault”, in other words a detected level of deceleration that is outside of an expected range, and/or has repeatedly transitioned above one or more deceleration thresholds. A deceleration fault can indicate damage (e.g. hooked end, bent shaft, and the like) to one or more microcoring elements 155 may have occurred (e.g. due to hitting bone or other hard surface during advancement). System 10 can be configured to prevent further use until inspection by an operator is performed (e.g. and treatment module 150 is replaced or confirmation of no damage is provided by the user). In some embodiments, system 10 is configured to monitor deceleration of a translating component of actuator 121z (e.g. monitor the deceleration of the associated coring elements 155) and to adjust (e.g. automatically adjust) the depth of penetration and/or other microcoring parameter as described herein. In some embodiments, system 10 is configured to monitor the deceleration of a translating component of actuator 121z (e.g. the component causing coring elements 155 to advance into and retract from tissue), and if the monitored deceleration exceeds (e.g. at any time) a maximum threshold, D_{MAX}, cause system 10 to enter an alarm or other alert state (“alert state” herein) in which the user (e.g. the clinician) is required to perform an action, such as: replacement of all or a portion of treatment module 150; inspection of treatment device 100 and/or another component of system 10; and/or performance of a safety and/or efficacy related task. In some embodiments, D_{MAX} comprises a deceleration level of no more than 75 g, such as no more than 60 g, such as a deceleration limit of approximately 50 g. In some embodiments, system 10 has multiple deceleration thresholds, such as a D_{MAX} (e.g. as described hereinabove), as well as one, two or more other, lower thresholds, such as a D₁, and/or a D₂ deceleration threshold, such as when D₂ is greater than D₁. System 10 (e.g. algorithm 525) can be configured to record the number of times actuator 121z exceeds either or both thresholds, and to enter an alert state (e.g. a state in which use of system 10 is stopped until further action is performed as described hereinabove), and/or to enter an alert state (e.g. a state in which use of system 10 can continue, but the user is notified of the exceeding of the threshold). In some embodiments, D1 comprises a threshold of no more than 40 g, such as no more than 35 g, or approximately 32 g, and system 10 enters an alert state if deceleration of actuator 121z exceeds D1. In some embodiments, system 10 enters an alert and/or an alert state (e.g. as described hereinabove) if a difference in deceleration (e.g. between two or more advancements of actuator 121z) exceeds a threshold (e.g. one advancement has a deceleration that exceeds a previous advancement by a threshold level). In some embodiments, monitoring of deceleration of actuator 121z is used to control and/or adjust (e.g. automatically adjust) the depth of penetration (also referred to as depth of advancement, or depth of insertion) of element 155.

In some embodiments, system 10 includes one or more sensors configured to monitor for inadequate communication (e.g. loss of communication) between two or more components of system 10 (e.g. between treatment device 100 and console 500). For example, algorithm 525 can monitor the signals produced by the one or more sensors, and can cause system 10 to enter an alert state if inadequate communication has been detected (e.g. loss of one form of communication has been detected).

System 10 (e.g. console 500 and/or treatment device 100) can be configured to operate in a closed loop mode, such as via a PID or other control module. In some embodiments, system 10 is configured to adjust (e.g. automatically adjust) the depth of penetration of coring elements 155 based on deceleration of actuator 121z (e.g. such as when system 10 records, stores, and/or otherwise monitors the deceleration of actuator 121z).

Accessories 90 can include various accessory components, such as a power cord (e.g. to attach to wall power), one or more filters, suction tubing, and/or other accessory components. Accessories 90 can include a footswitch, such as a user-controlled footswitch configured to initiate and/or stop microcoring or other function of system 10.

Functional element 599 of console 500 and/or functional element 199 of treatment device 100 can comprise a data transmission module, such as a cellular or other wireless transceiver, and/or a wired connection transceiver. System 10 can be configured to transmit system 10 use and/or other recorded information (e.g. data logs) to a remote site (e.g. the cloud, the system 10 manufacturer's location, a data collection service, and the like) via the transceiver, such as to collect, process, and/or analyze data collected by one or more systems 100 that are in use at one or more clinical settings.

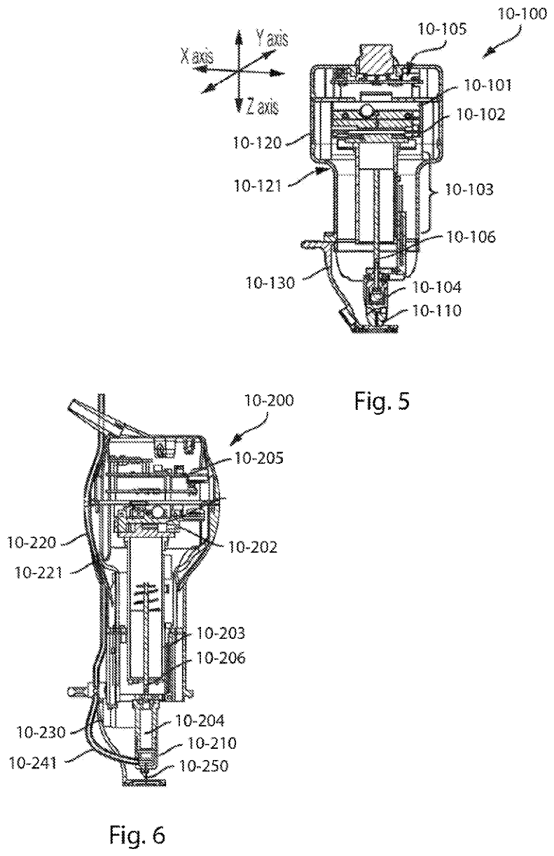

Referring now to FIGS. 5-34, tissue treatment systems, devices, and components are illustrated.

The present inventive concepts described herein may include a system that includes an apparatus for microcoring tissue of a subject (also referred to as “patient” herein). An example apparatus 10-100 is shown in FIG. 5, such as for use by a clinician or other user (“operator” or “user” herein). An apparatus 10-100 as described herein may include an actuation unit including one or more actuation mechanisms to drive a needle hub and/or a hollow needle into skin (e.g. in a z-direction) or across skin (e.g. in an x- and/or y-direction). In some embodiments, an actuation unit of the apparatus 10-100 may be or include one or more x-actuators (e.g. x-actuator 10-101), one or more y-actuators (e.g. y-actuator 10-102), and one or more z-actuators (e.g. z-actuator 10-103). In some embodiments, an actuation mechanism (e.g. z-actuator 10-103) may be connected to a needle hub mount (e.g. needle hub mount 10-104) for removably mounting a needle hub (e.g. needle hub 10-110) connected to one or more needles (not shown), such as via pushrod 10-106. In some embodiments, an apparatus for microcoring as described herein may be configured as a hand-held device that may be or include a hand piece comprising a hand piece shell (e.g. hand piece shell 10-121) encasing one or more components of an apparatus, such as actuators 10-101, 10-102, and/or 10-103, and/or other components (e.g. printed circuit board, PCB 10-105, which can be configured to control one or more actuators). A hand piece may include or may be removably connected to other components of an apparatus 10-100, such as a spacer (e.g. spacer 10-130).

An example apparatus 10-200 is shown in FIG. 6. Apparatus 10-200 of FIG. 6 includes an x-actuator 10-201, a y-actuator 10-202, and a z-actuator 10-203. Z-actuator 10-203 may be connected to a needle hub mount 10-204 for removably mounting a needle hub 10-210 including an example needle 10-250, such as via pushrod 10-206. An example apparatus 10-200 may include a hand piece (e.g. hand piece 10-220 shown), such as a hand piece comprising a hand piece shell 10-221 encasing one or more components of an apparatus, such as actuators 10-201, 10-202, and 10-203, and/or other components (e.g. printed circuit board (PCB) 10-205, which can be configured to control one or more actuators). A hand piece 10-220 may be removably connected to one or more components of a system, such as a spacer (e.g. spacer 10-230). The example system may comprise a low pressure or (partial) vacuum system including vacuum tubing 10-241 connected to a needle hub 10-210.

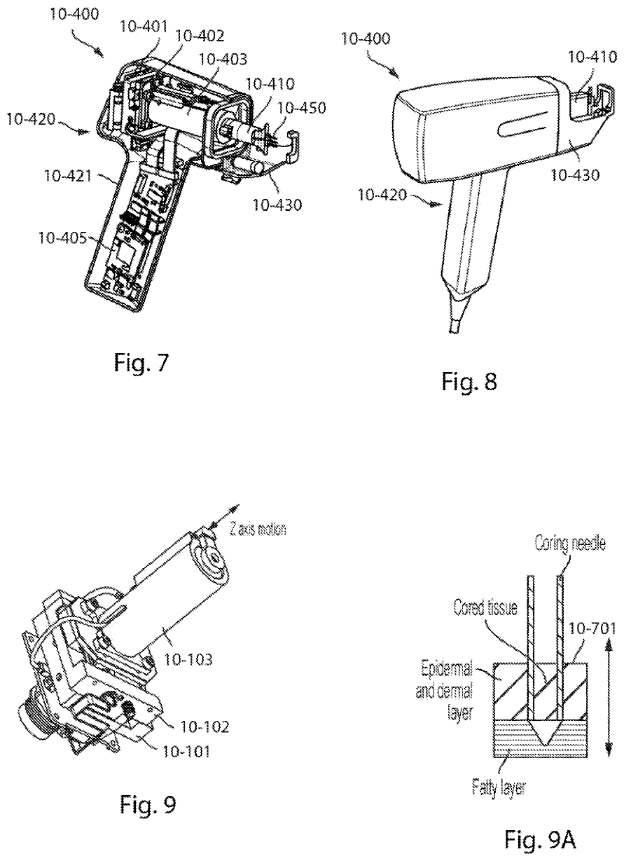

An example apparatus 10-400 is shown in FIG. 7. Apparatus 10-400 of FIG. 7 includes an x-actuator 10-401, a y-actuator 10-402, and a z-actuator 10-403. Z-actuator 10-403 may be connected to a needle hub mount (not shown) for removably mounting a needle hub 10-410 including one or more (e.g. three) example needles 10-450, such as via a pushrod (not shown). An example apparatus 10-400 may include a hand piece (e.g. hand piece 10-420), such as a hand piece comprising a hand piece shell 10-421 encasing one or more components of an apparatus, such as actuators 10-401, 10-402, and 10-403, and/or other components (e.g. a printed circuit board (PCB) 10-405, which can be configured to control one or more actuators). A hand piece 10-420 may be removably connected to one or more components of a system, such as a spacer (e.g. spacer 10-430). The example system may comprise a vacuum system including vacuum tubing (not shown) connected to a needle hub 10-410.

FIG. 8 shows an external view of apparatus 10-400. Apparatuses 10-100, 10-200, and 10-400 are non-limiting example embodiments of technologies described herein. One or more features or components of apparatuses 10-100, 10-200, and 10-400 may be used interchangeably.

In some embodiments, an actuation unit of the apparatus (e.g. an example actuation unit shown in FIG. 6) may include only x- and y-actuators (e.g. x-actuator 10-101, y-actuator 10-102) and/or a z-actuator 10-103. A z-actuator 10-103 (e.g. a voice coil, a solenoid, and/or a linear screw drive, disposed in z-axis housing) may be part of a needle assembly of the apparatus (e.g. a z-actuator and a needle hub). In some embodiments, x-, y-, and/or z-actuators may drive a needle hub and/or one or more hollow needles into and/or across a large area of skin surface in a relatively short amount of time compared to manual deployment of a hollow needle. In some embodiments, x-, y-, and/or z-actuators may drive a needle hub and/or one or more hollow needles into and/or across a small area of skin surface (e.g. a small area on the face, such as the area between the nose and the upper lip). In some embodiments, the x-, y-, and/or z-actuators may drive a needle hub and/or one or more hollow needles into and/or across multiple large and/or small areas of skin surface.

An example actuation unit as shown in FIG. 9 may include a z-actuator (e.g. a voice coil actuator), an x-actuator (e.g. an x-actuator stage comprising a linear screw drive), and a y-actuator (e.g. a y-actuator stage comprising a linear screw drive). In some embodiments, an x-actuator and a y-actuator have the same type of drive mechanisms. In some embodiments, an x-actuator and a y-actuator have different type of drive mechanisms. One or more actuators may be connected to a printed circuit board (e.g. as part of a control system), which may drive and/or control the actuators and/or receive feedback from the actuators (e.g. to provide closed-loop control of actuation, such as to a control system). In some embodiments, an x-actuator and a y-actuator (e.g. an x-actuator stage and a y-actuator stage) may be stacked, such as to form an x/y-stage. In some embodiments, a z-actuator may be mounted on a stack of an x-actuator and a y-actuator (an x/y-stage), for example a z-actuator may be mounted on an x-actuator, and the x-actuator may be mounted on a y-actuator. In some embodiments, a stack of an x-actuator and a y-actuator may be mounted in and/or on a hand piece shell. In some embodiments, an x-actuator and a y-actuator may be mounted separately in and/or on a hand piece shell, such as when the z-actuator is mounted and/or connected on an x-actuator and a y-actuator (e.g. on moveable tracks).

### Z-Actuation

A z-actuator (e.g. z-actuator 10-103, 10-203, or 10-403) may drive displacement of a needle hub and/or one or more hollow needles along an axis (e.g. a z-axis), such as to drive penetration into the skin by a hollow needle and/or retraction of the hollow needle after insertion (e.g. as shown in FIG. 9A). In some embodiments, a z-axis is substantially perpendicular to a skin surface 10-701 to be treated. In some embodiments, a z-axis is at an angle to a skin surface to be treated, for example at an angle of about 90 degrees, 80 degrees, 70 degrees, 60 degrees, 50 degrees, 40 degrees, 30 degrees, 20 degrees, or 10 degrees. In some example embodiments, coring at an angle other than substantially perpendicular to a surface of skin may increase size of a microcore, and/or a ratio of dermis/epidermis to fat.

In some embodiments, a z-actuator, (e.g. z-actuator 10-103, 10-203 or 10-403) may be located inside a hand piece (e.g. hand piece 10-120, 10-220, or 10-420), e.g. such as when encased by a hand piece shell (e.g. hand piece shell 10-121, 10-221, or 10-421). In some embodiments, a z-actuator may be located external to a hand piece shell, such as when the z-actuator is mechanically coupled to a needle hub and/or one or more hollow needles.

In some embodiments, a z-actuator may be connected to a needle hub through a mounting assembly. In some embodiments, a mounting assembly may include a pushrod (e.g. a z-axis pushrod connected to a voice coil actuator, such as pushrod 10-106 or 10-206 and a needle hub mount (e.g. needle hub mount 10-104 or 10-204). In some embodiments, a z-actuator (e.g. a voice coil actuator) is part of a needle assembly of an apparatus and may be detachably attached to a needle hub.

A z-actuator as described herein may be capable of operating at a high speed to minimize treatment time and deflection of skin tissue during the penetration of the hollow needle. In some embodiments, one actuation cycle in the z-direction may take from about 5 milliseconds to about 50 milliseconds (e.g. 5, 10, 15, 20, 25, 30, 35, 40, 45, or 50 milliseconds). In some embodiments, a z-actuator may take about 20 to about 35 milliseconds (e.g. 20, 21, 22, 23, 24, 25, 26, 27, 28, 29, 30, 31, 32, 33, 34, or 35 milliseconds) to travel about 20 mm to about 30 mm (e.g. 20, 21, 22, 23, 24, 25, 26, 27, 28, 29, or 30 mm) distally toward and/or into skin tissue. In some embodiments, a z-actuator may take about 25 milliseconds to about 30 milliseconds (e.g. 25, 26, 27, 28, 29, or 30 milliseconds) to travel about 23 mm distally toward and/or into skin tissue. In some embodiments, a z-actuator may take about 25 to about 35 milliseconds (e.g. 25, 26, 27, 28, 29, 30, 31, 32, 33, 34, or 35 milliseconds (e.g. 30 milliseconds)) to travel about 20 mm to about 30 mm (e.g. 20, 21, 22, 23, 24, 25, 26, 27, 28, 29, or 30 mm (e.g. 23 mm)) proximally from a penetration depth of about 20 mm to about 30 mm (e.g. 20, 21, 22, 23, 24, 25, 26, 27, 28, 29, or 30 mm (e.g. 23 mm)) into skin tissue. In some embodiments, a z-actuator may take about 30 milliseconds to travel about 23 mm proximally from a penetrated skin tissue.

A z-actuator as described herein may further be capable of operating with a certain insertion force and/or retraction force. In some embodiments, a force of about 0.5 N to about 20 N (e.g. 0.5 N to 0.75 N, 0.5 N to 1 N, 0.5 N to 1.25 N, 0.5 N to 1.5 N, 0.5 N to 2 N, 0.5 N to 5 N, 0.5 N to 10 N, 0.5 N to 12 N, 0.5 N to 15 N, 0.5 N to 20 N, 0.75 N to 1 N, 0.75 N to 1.25 N, 0.75 N to 1.5 N, 0.75 N to 2 N, 0.75 N to 5 N, 0.75 N to 10 N, 0.75 N to 12 N, 0.75 N to 15 N, 0.75 N to 20 N, 1 N to 1.25 N, 1 N to 1.5 N, 1 N to 2 N, 1 N to 5 N, 1 N to 10 N, 1 N to 12 N, 1 N to 15 N, 1 N to 20 N, 1.25 N to 1.5 N, 1.25 N to 2 N, 1.25 N to 5 N, 1.25 N to 10 N, 1.25 N to 12 N, 1.25 N to 15 N, 1.25 N to 20 N, 1.5 N to 2 N, 1.5 N to 5 N, 1.5 N to 10 N, 1.5 N to 12 N, 1.5 N to 15 N, 1.5 N to 20 N, 2 N to 5 N, 2 N to 10 N, 2 N to 12 N, 2 N to 15 N, 2 N to 20 N, 5 N to 10 N, 5 N to 12 N, 5 N to 15 N, 5 N to 20 N, 10 N to 12 N, 10 N to 15 N, 10 N to 20 N, 12 N to 15 N, 12 N to 20 N, or 15 N to 20 N) per hollow needle may be applied, such as to ensure insertion of one or more hollow needles into skin. In some embodiments, a force of about 10 N to 20 N (e.g. 15 N) per hollow needle may be applied, such as to ensure insertion of one or more hollow needles into the skin. Without intending to be bound by theory, insertion force may be inversely correlated with needle gauge. For example, a 24 gauge needle (e.g. a needle with an outer diameter of about 0.565 mm) may be operated with an insertion force of 12 N, while a 20 gauge needle (e.g. a needle with an outer diameter of about 0.9081 mm) may be operated with a higher insertion force. In some embodiments, an apparatus may include a feature and/or setting that may be used to control or change insertion force and/or retraction force of a hollow needle into and/or out of skin. In some embodiments, an adjustment implement, such as a scroll wheel on a user interface of the a base unit (e.g. a unit comprising at least a part of a control system), may be used to adjust an insertion force and/or a retraction force by the a hollow needle by physically adjusting (e.g. retracting) the hollow needle, such as adjusting position of a hollow needle relative to a distal end of an apparatus, such as when a z-actuator is fully retracted (e.g. by adjusting a position of a stationary base component of a z-actuator). In some embodiments, an adjustment implement, such as a scroll wheel on a user interface of a base unit, may be used to provide an electrical signal to a z-actuator to control insertion and/or retraction force. In some embodiments, a digital control unit or control system including a user interface of a base unit may control distance, velocity, force and/or timing of penetration into and/or retraction out of the skin by a hollow needle. Parameters such as insertion force and/or retraction force may be monitored. In some embodiments, a z-actuator is or comprises a voice coil that includes and/or is connected to a closed loop position and/or momentum/energy control system, as described herein.

In some embodiments, a z-actuator may provide position, velocity, acceleration, voice coil current, and/or voltage feedback signal to a z-axis position controller, such as a z-axis position controller that is part of a digital control unit as described herein. Feedback signals may be obtained from one or more sensors mounted on and/or integrated into a z-actuator. Feedback signals may be obtained from direct measurements, such as measurements of electric current and/or voltage entering or exiting a z-actuator (e.g. a voice coil). From these feedback signals, alone or in combination with known data (e.g. mass of a voice coil and/or needle assembly), a z-axis position controller (e.g. implemented on or as part of a digital control unit) may be used to measure and/or calculate a force required to insert/penetrate a subject's dermis and/or the force required to withdraw one or more coring needles from a subject's dermis.

A force required to penetrate dermal tissue may vary significantly between species, and may vary between subjects and/or skin types or areas to be treated. For example, abdominal dermal tissue may be thicker and/or tougher (harder to penetrate) than facial skin. Pig skin may be significantly thicker and/or tougher than human skin. A force required to penetrate dermal tissue may vary depending on number and/or configuration of needles used. Without intending to be bound by theory, as the number of needles on a single needle hub increases, a force required to penetrate the dermis (e.g. full thickness dermis) may increase proportionately. An amount of force or energy required to fully penetrate a subject's dermis may be measured and may provide an in-vivo indication of a patient's skin toughness, and/or an indication of the resilience provided by the skin pressing against a coring needle in direction of the z-axis. This information may be useful to evaluate skin characteristics of a subject (e.g. skin laxity). Lax dermal tissue may provide less resistance to a penetrating needle as compared to healthy and/or firm skin. A measurement of a force or energy required to penetrate a subject's dermal layer may provide useful diagnostic information to a clinician. For example, as a subject's skin quality improves with each coring treatment, a specific increase in skin toughness (increased resilience provided by subject's skin against a penetrating coring needle) may be monitored from treatment to treatment, providing an indication of improvement in skin quality.

In some embodiments, a number of electrical and/or mechanical parameters of a system of the present inventive concepts may be monitored before, during, and/or after coring, such as to determine tissue properties. Tissue properties may be determined based on data from one or more sensors and/or data from electrical and/or mechanical parameters of a z-actuator entering and/or exiting tissue, such as via voice coil and/or other actuator kinematics. Data may be used to characterize depth of tissue layers (e.g. dermis, epidermis, and/or fat), tissue quality of each layer (e.g. healthy, scarred, lax), and/or characterize location, shape, and/or volume of tissue features, such as scars or tumors (e.g. by combining said data with information of location (e.g. in an x-y plane of a treatment area) for each z-actuation).

In some embodiments, a coring process may be monitored to ensure successful coring and/or clearing of skin tissue from one or more hollow coring needles. In some embodiments, a measurement of force required to remove the coring needle from the patient's dermal layer may be used to indicate whether a core has been successfully withdrawn and/or excised, or not. A force required to retract one or more needles with a core (e.g. a new core) present in a lumen of one or more needles may be different from a force required to retract one or more needles without a core (e.g. a new core) present in a lumen of one or more needles. In some embodiments, a digital control unit may be used to monitor data received from a voice coil of a z-actuator (e.g. position, velocity, and/or acceleration of a voice coil), current draw, counter electromotive force (back EMF) and/or voltage, such as to derive successful coring information from voice coil data based on variation of force required to retract one or more needles from a tissue and/or variation of a velocity of needles being retracted from a tissue. In some embodiments, a radiofrequency (RF) energy may be applied to a needle, and output parameters may be monitored. Output parameters may vary based on presence of one or more cores inside a needle, thus indicating successful or unsuccessful coring. In some embodiments, radiofrequency energy may be applied to a needle to transfer energy to tissue, such as to improve coring and/or to core tissue selectively, such as by imparting a radiofrequency pulse when a needle is in contact with fat or septae. In some embodiments, heat may be generated in a tissue, such as through transfer of radiofrequency energy.

In some embodiments, amount and/or variation of pressure and/or flow rate in a fluid system in communication with one or more hollow needles may be monitored, such as by using one or more pressure gauges, to determine successful coring. In some embodiments, successful coring may be verified using visual inspection of one or more components of a fluid system in communication with one or more needles, such as by using one or more cameras. In some embodiments, electrical parameters in one or more components of a fluid system, such as capacitance and/or resistance, may be monitored to detect presence of one or more tissue cores. In some embodiments, an acoustic signal generated by an impact of one or more needles on skin tissue may be detected and monitored. An acoustic signal may vary depending on the presence of a core in one or more needles. In some embodiments, information from parameters monitored as described herein may also be used to detect worn or damaged needles and/or restricted and/or occluded needle lumens.

In some embodiments, if extraction of a core fails or is otherwise insufficient, a user (e.g. a clinician) may be informed of a coring deficiency, for example a digital control unit may receive and process data indicating unsuccessful coring as described above and may generate an output signal to a user interface, such as to display a warning to a user. A user may then examine one or more components of a system (e.g. a needle hub) to determine whether there is a clog and/or other obstruction. A user may select a deeper needle depth, for example to improve coring efficacy and/or efficiency. Without intending to be bound by theory, by monitoring the total energy required to withdraw one or more needles it may be possible to determine whether one or more cores were fully extracted. For example, if one or more needles fail to penetrate through full dermal thickness, for example into a fat layer, then one or more cores may not be released from the underlying (dermal) tissue. This partial penetration may result in a decrease in the force (energy) required to withdraw one or more coring needles.

In some embodiments, depth of needle penetration may be controlled, for example digitally controlled (e.g. using a digital control unit). In some embodiments, depth of needle penetration may be digitally controlled with a backup of one or more mechanical limit stops. In some embodiments, a digital control unit may be used to monitor voice coil data (e.g. position, velocity, and/or acceleration of a voice coil), current draw (e.g. indicating load on a needle), and/or voltage (e.g. to derive depth of penetration from voice coil data). This monitoring may allow detection of location of tissue and stop needle progression at a pre-selected depth, for example by accelerating or decelerating a voice coil or a moving component thereof.

In some embodiments, movement (displacement) of a z-actuator and/or of a (moving component of a) voice coil may be monitored, for example using one or more linear sensors (e.g. one or more encoders, such as a z-axis encoder) and/or one or more homing sensors (e.g. one or more optical sensors), for example to detect when a z-actuator is completely retracted, for example when a moveable component of a voice coil actuator is in the most proximal position away from a skin surface (e.g. linear displacement in direction of a skin surface is zero). In some embodiments, an amount of kinetic energy in a moving voice coil is matched to an amount of energy required to penetrate skin and/or reach a desired depth. In some embodiments, an open-loop control system may be used to control depth based on kinetic energy. In some embodiments, a reference accelerometer may be mounted on or connected to a different component of an apparatus or a hand piece, for example on the hand piece shell, to provide data to the digital control unit, such as to account for device movement.

Impact on hard tissue may occur as a result of over-penetration, for example penetration beyond a dermal layer and/or subcutaneous fat layer. In some embodiments, a controller (e.g. a digital control unit) may be used to monitor discrepancies between commanded z-axis position and actual z-axis position of a z-actuator and/or a voice coil, and/or may be used to monitor deceleration of the z-actuator and/or a voice coil. In some instances, discrepancies between commanded z-axis position and actual z-axis position may occur, for example due to a needle impacting an impenetrable structure prior to reaching commanded depth. In some embodiments, if such a discrepancy may be detected and/or if deceleration exceeds a certain threshold, a warning notice may be conveyed to a user (e.g. by a digital processing unit via a display), for example if deceleration and/or the amplitude of an acceleration/deceleration curve exceeds a certain threshold of about, for example, 10 m/s², 20 m/s², 30 m/s², 40 m/s², 50 m/s², 60 m/s², 70 m/s², 80 m/s², 90 m/s², 100 m/s², 200 m/s², 300 m/s², 400 m/s², 500 m/s², or 1000 m/s².

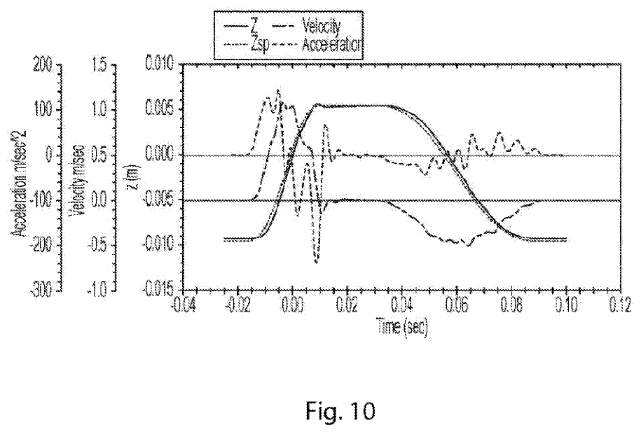

FIG. 10 shows an example plot of voice coil velocity, position, and acceleration against time during an example normal coring procedure. The commanded z-axis position matches the actual z-axis position. Also, deceleration is less than about 250 m/s².

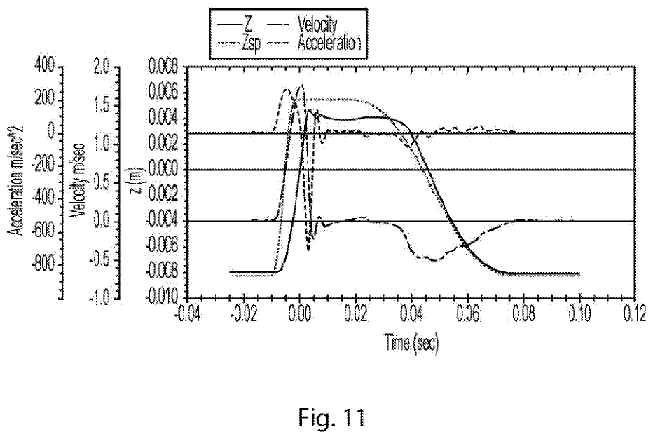

FIG. 11 shows an example plot of voice coil velocity, position, and acceleration against time before, during, and after a coring procedure with excessive over-penetration and contact with hard tissue resulting in a deceleration at impact of about 600 m/s². In this example, a needle tip was severely damaged. In some embodiments, a digital control unit may be used to monitor deceleration and may be used to provide a fault notice to a user, for example via a display. Measured decelerations greater than a certain threshold (e.g. a threshold of 10 m/s², 20 m/s², 30 m/s², 40 m/s², 50 m/s², 60 m/s², 70 m/s², 80 m/s², 90 m/s², 100 m/s², 200 m/s², 300 m/s², 400 m/s², 500 m/s², or 1000 m/s²) may result in termination of a needle lifetime. In some embodiments, a needle hub may be identified, for example upon mounting on a needle hub mount, such as through a signal received by a digital control unit from a Radio Frequency Identification (RFID) chip located on or in a needle hub. In some embodiments, a digital control unit may be used to block use of an apparatus (e.g. block actuation of a z-actuator), until a needle hub including one or more damaged needles is replaced, for example as indicated by the removal of the RFID chip associated with a (damaged) needle hub and mounting of a needle hub with a different RFID chip.

### Depth Control

In some embodiments, an apparatus of the present inventive concepts, for example apparatus 10-100, 10-200, or 10-400, may include one or more features or settings that may be used to control or change the depth of penetration of a hollow needle into the skin, for example by controlling one or more parameters of a z-actuator (e.g. z-actuator 10-103, 10-203, or 10-403). In some embodiments, an adjustment implement, such as a scroll wheel on a user interface of a base unit, may be used to adjust an allowed depth of penetration by a hollow needle into skin. In some embodiments, an allowed depth adjustment may be carried out by physically adjusting (e.g. retracting) a hollow needle, for example by adjusting position of a hollow needle relative to a distal end of an apparatus, such as when a z-actuator is fully retracted (at a most proximal position of an actuation cycle), such as by adjusting a position of a stationary base component of a z-actuator. In some embodiments, an adjustment implement, such as a scroll wheel on a user interface of a base unit, may be used to provide an electrical signal to a z-actuator to control depth of penetration. In some embodiments, a digital control unit including a user interface of a base unit may control depth and/or timing of penetration into and retraction out of skin by a hollow needle. For example, an operator may program a computer component of a base unit to require a certain displacement of a needle hub and/or a hollow needle into skin based upon an area being treated. A z-actuator as described herein may be programmed or otherwise set to displace a hollow needle up to about, for example, 10 mm into thick skin (e.g. on a patient's back or into scar tissue), or about, for example, 1 mm into thin skin (e.g. on a patient's cheeks). A z-actuator as described herein may be programmed or otherwise set to displace a hollow needle to extend (i) into a dermal layer, (ii) through the entire dermal layer to the junction of the dermal layer and the subcutaneous fat layer, and/or (iii) into the subcutaneous fat layer.

In some embodiments, a feedback and/or depth control system that may be used with the technologies described herein (e.g. apparatuses 10-100, 10-200, or 10-400) may include an electrically insulated needle. In some embodiments, a coring needle may be electrically insulated (e.g. an external surface of the needle may be electrically insulated, such as by an insulating coating) except for a distal tip, for example the needle may not be insulated (exposed) along a length of about 0.2 mm, 0.5 mm, 0.7 mm, 1 mm, 2 mm, 3 mm, 4 mm, or 5 mm from a distal end of a needle for contacting skin. An electrical signal (e.g. an RF signal) may be applied to a needle having a tip and an insulated lumen or body. Electrical feedback (e.g. a change in voltage, current, and/or impedance) from the needle tip may be monitored as the needle penetrates skin of a subject. Without intending to be bound by theory, once a needle passes through a dermal layer and begins entry into a fat layer, a measurable change in impedance detected may occur at the tip, for example due to a difference in electrical properties between tissue types. In some embodiments, this change in impedance may be used as z-axis position/depth indicator and may be used for feedback, such as when transmitted to a digital control unit, which in turn may use impedance information to generate or trigger a signal to a z-actuator, for example to control or adjust depth of penetration. In some embodiments, once a desired full dermal thickness depth has been reached by a needle tip, impedance feedback to a digital control unit may cause the unit to signal a z-actuator to stop and reverse (withdraw) from a patient's dermis.

In some embodiments, a system as described herein may include a control system, for example a digital control unit, that may be used to monitor voice coil data, such as to monitor position, velocity, acceleration, current draw, and/or voltage. A digital control unit may be used to control (e.g. accelerate or decelerate) a voice coil of a z-actuator based on pre-programmed commands and signals, and/or based on signals from a depth control system, for example a depth control system including an electrically insulated needle. In some embodiments, voice coil actuator and/or z-actuator movement may be monitored using a linear sensor (e.g. an encoder) and/or a homing sensor (e.g. an optical sensor) that may detect when a moveable component of a voice coil actuator or z-actuator is completely retracted away from a skin surface (e.g. is at a most distal position in an actuation cycle). In some embodiments, a vision system, for example a system including a camera, may be used to monitor needle travel. A reference accelerometer in and/or on an apparatus or hand piece (e.g. in and/or on a hand piece shell) may provide input data to a digital control unit, for example to account for device movement. In some embodiments, a digital control unit may be programmed to match an amount of kinetic energy in a voice coil to an energy required for a needle hub to reach a certain distance, for example for a needle to reach a certain depth.

In some embodiments, a needle may be advanced (e.g. further advanced) until an exposed RF tip is disposed entirely within a fat layer, and/or until the RF tip has reached a predetermined depth in a fat layer (e.g. 1 mm, 2 mm, or 3 mm depth of fat layer). This configuration of advancement may be useful for applications where it is desired to remove fat as well as skin cores. In some embodiments, a hypodermic needle (e.g. a needle of less than 1 mm in internal diameter) which is not intended to core skin, may be advanced into and through the dermis of a subject and into a fat layer below, which may result in a hole through the subject's dermis, but not a core. In some embodiments, fat and/or other tissue beneath the dermis may be withdrawn via a needle lumen, for example when configured to perform a liposuction procedure. In some embodiments, a signal from an RF tip that a fat layer was reached may be used as an input signal to a digital control unit that may be used to activate a tissue suction mechanism.

In some embodiments, an apparatus of the present inventive concepts may include or may be connected to one or more depth control systems that may include one or more skin surface and/or layer detection technologies. Skin surface or skin layer detection technologies may include systems and/or methods to monitor capacitance in a needle and detect changes therein to infer needle position/depth relative to a skin layer. Skin surface or skin layer detection technologies may include acoustic technologies, for example a microphone that may be used to ‘hear’ (i.e. audibly detect) the impact of one or more needles on a skin surface. Skin surface or skin layer detection technologies may include visual systems (e.g. one or more cameras) to detect and/or monitor skin surface location and/or needle/voice coil travel.

In some embodiments, a depth control system may include one or more technologies for detection of a dermal/fat interface, for example to control (e.g. stop) needle progression. Capacitance changes from air to dermis to fat may be detected and/or monitored using technologies analogous to technologies for impedance detection and/or monitoring, for example using one or more insulated or partially insulated needles, such as by using polyvinylidene fluoride (PVDF) as an insulating material.

In some embodiments, a depth control system may include one or more technologies that employ ultrasound, optical coherence tomography (OCT), and/or other acoustic or vision-based technology to assess depth of penetration by one or more needles, such as penetration of the fat/dermal interface. In some embodiments, dermal layer thickness may be determined by evaluating a previously removed core by vision, acoustic, and/or electrical systems or methods.

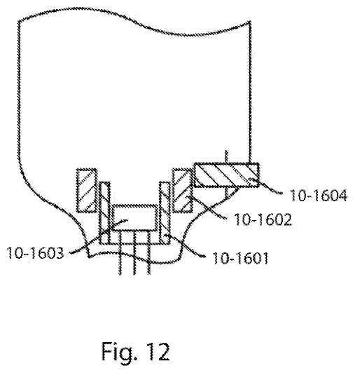

In some embodiments, mechanical depth control technologies may be used with the technologies described herein. Mechanical depth control technologies may include one or more depth control spacers, for example depth control spacer elements attached to a spacer frame as described herein, and/or other movement limitation implements that may limit z-actuation of a needle hub. In some embodiments, mechanical depth control technologies may be used alone or in combination with electrical technologies, for example as described above. FIG. 12 shows an example embodiment of an example apparatus as described herein including a mechanism, for example an internal threaded mechanism, to raise and/or lower (e.g. relative to a skin surface during operation) an actuation unit that may be or include a z-actuator (e.g. z-actuator 10-1603) and/or a moveable component of a voice coil actuator. In some embodiments, an internal threaded mechanism is or includes a rack and pinion or rack and worm arrangement (e.g. rack 10-1601 and worm 10-1602). In some embodiments, an internal threaded mechanism may be manually actuated (e.g. through a wheel, for example on a hand piece shell, such as wheel 10-1604), or may be actuated through a motor.

### Recoil Compensator

As a z-axis voice coil (or moving component thereof) accelerates and/or decelerates, a counter force may be imparted to an apparatus, including, for example, a hand piece (e.g. hand piece 10-120, 10-220, or 10-420) and/or hand piece shell (e.g. hand piece shell 10-121, 10-221, or 10-421) encasing an actuation unit comprising a z-actuator. A hand piece may be held by a user operator and may be configured for optimized ergonomics. In some embodiments, a hand piece and its components (e.g. a hand piece shell) are made as light as practical, such as for user comfort. Without intending to be bound by theory, a lower mass of the apparatus may worsen the recoil effect felt in the hand piece due to reduced inertia of the apparatus. In some embodiments, multiple needles, such as a needle array, may be used. The greater the number of needles on a given needle hub, the correspondingly greater acceleration may be required to drive the needles into or through the patient's dermis (e.g. to obtain a full thickness core), which may worsen user-felt recoil. An apparatus as described herein, for example an apparatus equipped with an ultra-light hand piece and/or a needle hub with multiple needles, may benefit from a recoil compensating mechanism, which may improve user experience and/or positional stability of a hand piece (e.g. by moving a mass counter to a z-axis stroke and cancelling or diminishing user felt recoil).

In some embodiments, a z-actuator may include multiple voice coil actuators. In some embodiments, a z-actuator comprises dual countering voice coils and/or voice coil actuators arranged along their axis of movement. Dual countering voice coils may be used such that one voice coil (and/or a moving component thereof) cancels or reduces an effect of a change in momentum of the other voice coil (and/or a moving component thereof) during operation. In some embodiments, a z-actuator may include or may be connected to a recoil compensator, for example a counterbalance mass to reduce the effect of a change in momentum of a voice coil (and/or a moving component thereof) during operation.

In some embodiments, an apparatus of the present inventive concepts may include a hand piece accelerometer (e.g. mounted on or connected to the hand piece shell), which may provide feedback to a system (e.g. a digital control unit) including, for example, a z-axis counter mass controller. A z-axis counter mass controller may be used to minimize accelerations detected by the hand piece accelerometer. In some embodiments, a z-axis counter mass controller may include a counter mass weight, for example a piece of metal, which may be moveably mounted in and/or on the apparatus (e.g. in and/or on a hand piece and/or a hand piece shell) in a direction substantially parallel and/or opposite to the direction of motion of a voice coil (and/or a moving component thereof) of a z-actuator and/or a needle hub displaced by the z-actuator. A z-axis counter mass controller may include a motor for moving the counter mass weight and may include an electronic control system. In some embodiments, an electronic control system (e.g. a digital control unit) may be used to monitor movement of the apparatus (e.g. the hand piece, such as with monitoring based on data obtained from the accelerometer), and to move the counter mass weight in a direction opposite to the direction of movement of the apparatus (e.g. the hand piece). In some embodiments, an electronic control system may be used to monitor movement of a needle hub and/or voice coil of a z-actuator and to move the counter mass weight in a direction opposite to the direction of movement of the needle hub and/or voice coil. In some embodiments, the z-axis counter mass weight may be of equal weight and may be moved with equal but opposite acceleration and/or velocity as the voice coil (and/or a moving component thereof) of a z-actuator, which may cancel recoil caused by movement of the z-axis voice coil, without acceleration feedback from an accelerometer. In some embodiments, a counter mass weight may travel the substantially same distance at the substantially same velocity as a voice coil (or moving component thereof) of a z-actuator or a needle hub displaced by a z-actuator. In some embodiments, a counter mass weight may act to reduce rather than cancel recoil felt by an operator of the apparatus. In an example embodiment, a z-axis counter mass weight may travel in a direction opposite to the direction of movement of a voice coil (and/or a moving component thereof) of a z-actuator or a needle hub displaced by a z-actuator, but only by a fraction of the distance of movement of a (moving component of a) voice coil of a z-actuator or a needle hub displaced by a z-actuator. This travel may reduce the worst “edges” of felt recoil, which may occur at an end of travel distance of a voice coil (and/or a moving component thereof) of a z-actuator or a needle hub displaced by a z-actuator, which may be the period of maximum acceleration and/or deceleration. In some embodiments, a recoil compensating counter mass weight may be driven by a voice coil actuator substantially similar to the z-actuator, wherein the recoil compensating voice coil actuator is arranged to move the reciprocate counter mass weight in a direction opposite to the direction of travel of a voice coil (or moving component thereof) of a z-actuator or a needle hub displaced by a z-actuator.

In some embodiments, a z-actuator may be configured to maintain an apparatus or a component thereof at a low temperature (e.g. less than about 43° C., such as less than about 43, 42, 41, 40, 39, 38, 37, 36, or 35° C.) to avoid subject and/or user discomfort and/or to avoid damage to the skin tissue (e.g. collagen in the skin tissue is sensitive to high temperatures, such as temperatures above 40° C.). Actuator types having characteristics for maintaining a low temperature include voice coil actuators, pneumatic actuators, electromagnetic actuators, motors with cams, motors with lead screws (e.g. stepper motors), and piezoelectric actuators. In some embodiments, a low temperature z-actuator is a voice coil actuator.

### XY-Actuator

In some embodiments, an apparatus of the present inventive concepts may include an “x” and/or a “y” actuator (e.g. an x/y actuator) for translating a needle hub and/or one or more hollow needles across skin, for example x-actuator 10-101, 10-201, or 10-401 and/or y-actuator 10-102, 10-202, or 10-402. An x/y-actuator may be used to establish skin treatment coverage. In some embodiments, an x/y-actuator may have a relatively small displacement range (e.g. maximum distance between a first x/y position and a second x/y position), such as less than about 10 mm (e.g. 10, 9, 8, 7, 6, 5, 4, 3, 2, or 1 mm). In some embodiments, an x/y-actuator may have a relatively large displacement range (e.g. up to about 30 mm). An x/y-actuator may operate with high positional accuracy (e.g. distance between a selected position and actual position, for example of a hollow needle). For example, an x/y-actuator may position a hollow needle to penetrate skin within a 30 pm radius (e.g. within 30, 25, 20, 15, 10, or 5 pm) of a selected position. An x/y-actuator may operate with high position accuracy that may allow continuous treatment across a treatment area. High position accuracy may provide the ability to re-enter a hole previously created and/or repeat coring at a position previously targeted, for example if coring was not achieved completely. In some embodiments, a needle may re-enter a hole previously created or previously targeted by the same needle, for example without translation in the x or y direction between the two entries. In some embodiments, a needle may enter a hole previously created or previously targeted by a different needle. In some embodiments, to deliver a drug or other substance to the hole, a needle may re-enter a hole previously created.

A treatment area may be a skin area that contains multiple treatment sites, for example a 3 cm by 3 cm treatment area containing nine 1 cm² treatment sites. An x/y-actuator may facilitate movement of a needle hub and/or one or more hollow needles of an apparatus from one treatment site to an adjacent treatment site within a treatment area. An x/y-actuator may facilitate movement of a needle hub and/or one or more hollow needles of an apparatus within each treatment site. An x/y-actuator may operate with high position accuracy that may avoid gaps between adjacent treatment sites in a treatment area and/or avoid overlaps between adjacent treatment sites in a treatment area. In some embodiments, an x/y actuator may enable creation of different hole patterns (e.g. multiple hole patterns with different penetration geometries). In some embodiments, a hole pattern may be regular or irregular, uniform or non-uniform. Regular patterns include rows and/or arrays of equally spaced holes. Irregular patterns include random patterns. Uniform patterns include rectangular or arrays of equally spaced holes. Non-uniform patterns include arrays with differently spaced holes. In some embodiments, a pattern can be pre-set or pre-programmed, for example to match tissue conditions and/or desired treatment effect. In some embodiments, a pattern may be altered or modified during operation of the device. Examples of array patterns that may be generated with the technologies described herein are described in detail below.

An x/y-actuator may also operate at a relatively high speed to reduce treatment time. In some embodiments, one actuation cycle in the x- and/or y-direction may take from about 50 milliseconds to about 250 milliseconds (e.g. 50, 75, 100, 125, 150, 175, 200, 225, or 250 milliseconds). In some embodiments, one actuation cycle in the x- and/or y-direction may take about 120 milliseconds to about 160 milliseconds (e.g. 120, 125, 130, 135, 140, 145, 150, 155, or 160 milliseconds, such as about 140 milliseconds). In some embodiments, one actuation cycle in the x- and/or y-direction may take about 120 milliseconds to about 160 milliseconds (e.g. 120, 125, 130, 135, 140, 145, 150, 155, or 160 milliseconds, such as about 140 milliseconds) to travel about 0.6 mm to about 1 mm (e.g. 0.6, 0.65, 0.7, 0.75, 0.8, 0.85, 0.9, 0.95, or 1 mm). In some embodiments, one actuation cycle in the x- and/or y-direction may take about 140 milliseconds to travel about 0.833 mm.

In some embodiments, an x/y-actuator may be capable of operating with a force of about 0.5 N to about 20 N (e.g. 0.5 N to 0.75 N, 0.5 N to 1 N, 0.5 N to 1.25 N, 0.5 N to 1.5 N, 0.5 N to 2 N, 0.5 N to 5 N, 0.5 N to 10 N, 0.5 N to 12 N, 0.5 N to 15 N, 0.5 N to 20 N, 0.75 N to 1 N, 0.75 N to 1.25 N, 0.75 N to 1.5 N, 0.75 N to 2 N, 0.75 N to 5 N, 0.75 N to 10 N, 0.75 N to 12 N, 0.75 N to 15 N, 0.75 N to 20 N, 1 N to 1.25 N, 1 N to 1.5 N, 1 N to 2 N, 1 N to 5 N, 1 N to 10 N, 1 N to 12 N, 1 N to 15 N, 1 N to 20 N, 1.25 N to 1.5 N, 1.25 N to 2 N, 1.25 N to 5 N, 1.25 N to 10 N, 1.25 N to 12 N, 1.25 N to 15 N, 1.25 N to 20 N, 1.5 N to 2 N, 1.5 N to 5 N, 1.5 N to 10 N, 1.5 N to 12 N, 1.5 N to 15 N, 1.5 N to 20 N, 2 N to 5 N, 2 N to 10 N, 2 N to 12 N, 2 N to 15 N, 2 N to 20 N, 5 N to 10 N, 5 N to 12 N, 5 N to 15 N, 5 N to 20 N, 10 N to 12 N, 10 N to 15 N, 10 N to 20 N, 12 N to 15 N, 12 N to 20 N, or 15 N to 20 N) per hollow needle can be applied to translate the needle across the skin. In some embodiments, a force of about 5 N to 15 N (e.g. 10 N) per hollow needle may be applied to translate a needle across skin.

An x/y-actuator may be configured to maintain an apparatus or a component thereof at a low temperature (e.g. less than about 43° C., such as less than about 43, 42, 41, 40, 39, 38, 37, 36, or 35° C.) in order to avoid raising the apparatus temperature to a level that could cause subject and/or user discomfort. Actuator types having characteristics for maintaining a low temperature include voice coil actuators, pneumatic actuators, electromagnetic actuators, motors with cams, piezoelectric actuators, and motors with lead screws (e.g. stepper motors). In some embodiments, an x/y-actuator is a stepper motor with a lead screw.

In some embodiments, one or more components of an apparatus of the present inventive concepts may be selected or designed to secure a needle hub and/or one or more hollow needles and/or prevent or minimize angular movement (e.g. wobbling) of the hollow needle(s). In some embodiments, an x-, y-, and/or z-actuator may operate without causing any significant angular movement (e.g. wobbling) of a needle hub and/or one or more hollow needles. In some embodiments, a z-actuator may insert and/or withdraw one or more hollow needles in a linear fashion without any significant angular movement (e.g. wobbling) of the one or more hollow needles. A hollow needle may be secured to a needle hub so as to minimize or reduce angular movement of needle(s) during insertion to less than 5 degrees, such as less than 4, 3, or 2 degrees. An angular movement of a needle during insertion of 1.0 to 1.5 degrees may be within nominal tolerances, whereas an angular movement of the needle during insertion of 4.0 to 5.0 degrees or more may need to be avoided, if possible. In some embodiments, components that join one or more hollow needle(s) to other components of the needle assembly, for example a needle hub, may be designed with low mechanical tolerances to firmly secure the one or more hollow needles. This tolerance requirement may reduce prevalence of and/or may lower the risk of destabilization and/or reduction in the structural integrity of hollow needle(s) that may result from repeated use. Firmly securing needle(s) may prevent and/or minimize dulling, bending, and curling of needle tip(s) that could reduce the effectiveness of the needle(s). Firmly securing needle(s) may also reduce the risk of over-striking (e.g. striking a hole produced by a needle again).

In some embodiments, actuators, for example z-, x-, and/or y-actuators, may be activated independently and/or collectively by one or more buttons, keys, toggles, switches, screws, dials, cursors, spin-wheels, and/or other activatable components. In some embodiments, each of the z-, x-, and/or y-actuators can be separately controlled (e.g. using separate activation components, such as a button, or by using separate controls in a user interface). In some embodiments, an apparatus includes a multiplexer, for example to select one or more input signals or output signals, such as from or to an actuator or sensor, and to transmit a signal in a single line.

### Rotary Stage

In some embodiments, an apparatus and/or an actuation unit as described herein may be or include a rotary stage, for example to rotate a needle hub around an axis perpendicular to a surface of skin to be treated, such as around a z-axis. A rotary stage may include one or more motors and/or actuators, such as an electrical motor a stepper motor, and the like. In some embodiments, a rotary stage is or comprises a z-actuator (e.g. as described above) and/or a rotation mechanism.

In some embodiments, a movement of or by a z-actuator may cause a needle hub and/or one or more needles (e.g. a needle array) to rotate, for example by about 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or 90 degrees, and/or to rotate by about 100 degrees, 110 degrees, 120 degrees, 130 degrees, 140 degrees, 150 degrees, 160 degrees, 170 degrees, 180 degrees, 190 degrees, 200 degrees, 210 degrees, 220 degrees, 230 degrees, 240 degrees, 250 degrees, 260 degrees, 270 degrees, 280 degrees, 290 degrees, 300 degrees, 310 degrees, 320 degrees, 330 degrees, 340 degrees, 350 degrees, or 360 degrees. In some embodiments, each movement of or by a z-actuator may cause a needle hub and/or one or more needles (e.g. a needle array) to rotate, for example around a z-axis of a z-actuator. In some embodiments, a 3×3 needle array may be rotated by 90 degrees during each actuation of a z-actuator. In some embodiments, an apparatus may be used or configured for concurrent patterning, for example needles may act on the two or more different quadrants or equivalent (e.g. sectors of any size or shape). An apparatus as described herein may be configured for any number of strokes (insertions and retractions of one or more needles) to complete a pattern of holes.

In some embodiments, a rotation mechanism may be used that includes a single planar translation mechanism, for example translation along a radius of a circle. Instead of encoding a position of a needle hub and/or z-actuator in a Cartesian coordinate system (x, y), a position of a needle hub and/or a z-actuator may be encoded in polar coordinates (e.g. radius r, angle theta). In some embodiments, use of a rotation mechanism with two degrees of freedom may eliminate the need for x/y-translation and thus a need for an x-actuator and/or a y-actuator. This elimination may lead to reduced weight of an apparatus, reduced size of a hand piece, and/or reduced cost. Reduction of hand piece size, for example hand piece shell diameter reduction, may be an advantage to users with respect to ease of use of an apparatus as described herein.

### Needle Hub

The technologies described herein may include a system and/or apparatus that includes a needle hub. In some embodiments, a needle hub may be or include a needle hub assembly comprising one or more needle joints, for example joints configured to receive and/or hold one or more needles (e.g. hollow needles). In some embodiments, a needle hub may include a first lumen having a wall, a first end and a second end. A first lumen may include, or may be in fluid communication with, a lumen of a hollow needle, for example where the first end of the first lumen is at a distal end of the hollow needle for contacting skin.

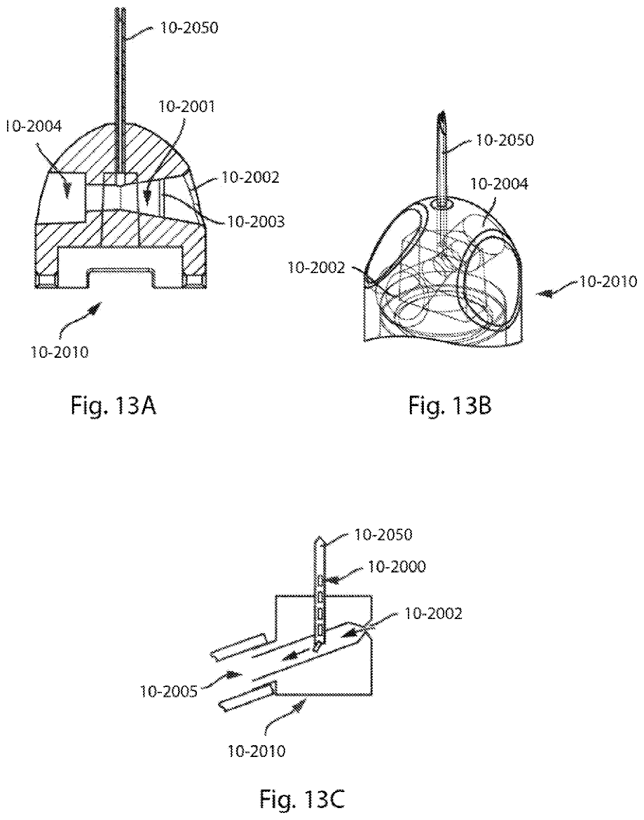

In some embodiments, a needle hub may be or include a needle hub assembly including two or more lumens, for example two lumens in fluid communication with each other. In some embodiments, a needle hub may include a second lumen having a wall, a first end, and a second end. In some embodiments, a second lumen may be in fluid communication with a first lumen, for example where the first lumen may include, or may be in fluid communication with, a lumen of a hollow needle. In some embodiments, a first lumen may be connected to a second lumen forming a junction such that the second end of the first lumen forms an opening in the wall of the second lumen. This may facilitate clearing of material, for example skin cores, from a first lumen (e.g. from an example hollow needle), as described further below. An example needle hub 10-2010 with two lumens is shown in FIGS. 13A and 13B.

First and second lumens and/or junctions between a first and second lumen may have any shape and/or configuration. In some embodiments, each of the first lumen and the second lumen may be substantially straight, and the first lumen may be substantially perpendicular to the second lumen forming a T-junction. In some embodiments, the first and second lumen may be connected at an angle, for example at an angle of about 0 degrees, 10 degrees, 20 degrees, 30 degrees, 40 degrees, 50 degrees, 60 degrees, 70 degrees, 80 degrees, or 90 degrees. In some embodiments, one or both of a first lumen and a second lumen may be curved and/or include a substantially straight and/or a curved section. In some embodiments, a lumen, for example one or both of a first lumen and a second lumen, may have a constant diameter along a length of a lumen or may have a diameter varying along a length of a lumen. A lumen may have any cross-sectional shape, such as circular, square, oval, rectangular, angular, or any combination thereof.

In some embodiments, a first lumen may include a lumen of a hollow needle (e.g. needle 10-2050), for example when the first end of the first lumen is at a distal end of the hollow needle for contacting skin. In some embodiments, the first end of the second lumen may be or include a fluid intake nozzle (e.g. an air intake nozzle 10-2001). In some embodiments, a fluid intake nozzle may be or include a convergent nozzle, a divergent nozzle, a convergent-divergent nozzle, a cylindrical nozzle, and/or a frusto-conical nozzle. In some embodiments, a second lumen or fluid intake nozzle may include a filter (e.g. filter 10-2003) to remove impurities (e.g. dust) from fluid traversing the nozzle. In some embodiments, a first end of the second lumen (e.g. a nozzle) may be exposed to ambient atmosphere (e.g. at intake 10-2002). In some embodiments, a first end of the second lumen (e.g. a nozzle 10-2001) may be connected to a fluid conduit (e.g. at outlet 10-2004) at the second end of the second lumen. In some embodiments, a first end (e.g. a nozzle 10-2001) of the second lumen may not be connected to a fluid conduit.

### Core Clearing

A needle hub as described herein may be used for or to facilitate removal of tissue from a hollow needle. In some embodiments, a needle hub may be connected to or may be part of a fluid system, for example a fluid-based core clearing system, that may be used to facilitate removal of tissue (e.g. one or more skin cores) from one or more needles. As a coring needle is driven into or through the dermis (and/or into a fat layer below) of a subject, skin tissue (e.g. one or more full thickness skin cores) is driven up into a needle lumen. During repeated operation with the same needle, multiple skin cores may stack up inside a lumen of a hollow needle and/or a first lumen of a needle hub, and these cores may compress together. Repeated operation may lead to one or more skin cores filling up a lumen of a hollow needle and/or a first lumen of a needle hub. Repeated operation of the same needle may lead to one or more skin cores being pushed out of an opening in the lumen of a hollow needle and/or out of a first lumen of a needle hub (e.g. out of a second end of the first lumen). In some embodiments, a first lumen may be connected to a second lumen as described above, for example where a first lumen is connected to a second lumen forming a junction such that the second end of the first lumen forms an opening in the wall of the second lumen. In some embodiments, a second end of a second lumen may be connected to a fluid conduit such that when low pressure or (partial) vacuum is applied to the conduit, low pressure or (partial) vacuum is induced in the first lumen and second lumen, for example such that fluid may be drawn into the second lumen through the first end of the second lumen.

Without intending to be bound by theory, once a core begins to emerge from a first lumen, for example a lumen of a hollow needle, and enter a second lumen, the core may be exposed to cross fluid flow in the second lumen (e.g. an airstream, such as a high velocity airstream, such as a (near) supersonic airstream) and associated drag force. Any fluid may be used, for example any gas (e.g. air, carbon dioxide, or nitrogen gas) or any liquid (e.g. water, saline, an aqueous solution, or oil), or any combination thereof. In some embodiments, fluid flow (e.g. airstream) in a second lumen exerts a lateral (drag) force on a side of a first core emerging from a first lumen, which may pull the core from the first lumen (e.g. as the core is flexible and may bend, thus translating the force acting on the side of the core into a tensional force). In some embodiments, during repeated operation of the same needle, multiple cores may enter a lumen of a hollow needle and/or a first lumen. One or more cores stacking behind (e.g. distally along a first lumen, such as a hollow needle lumen) an emerging core (e.g. a first core) in a first lumen may push the emerging core into a second lumen. In some embodiments, a core (e.g. an emerging core) may be exposed to both a lateral force from a fluid stream and force exerted from one or more cores stacking behind the emerging core. In some implementations, a suction force may act on a first lumen (e.g. a lumen of a hollow needle), which may cause one or more cores to be sucked from the first lumen, for example into a second lumen. FIG. 13C is a diagram illustrating an example core clearing procedure (e.g. in a needle hub 10-2010) wherein air is drawn into a second lumen through an air intake 10-2002 by means of a vacuum source downstream of the second lumen in fluid connection with needle hub 10-2010 through a vacuum line 10-2005. One or more skin cores 10-2000 may be drawn from a first lumen into the second lumen.

In some embodiments, a lumen, for example a second lumen, may be configured as a Venturi like nozzle. Fluid (e.g. air) may be drawn through a nozzle, for example a fluid intake nozzle at a first end of the second lumen. In some embodiments, a fluid intake nozzle may include a filter, nozzle inlet, and/or a cross sectional constriction followed by a larger cross section tubing, for example a second lumen may be configured as a convergent-divergent duct. In some embodiments, constant airflow may be drawn through the fluid intake nozzle. Fluid flow (e.g. air flow) may accelerate through a convergent part of the lumen, reaching a maximum air velocity at a constriction of a convergent-divergent duct, for example the smallest cross sectional area of the lumen. A first lumen may be connected to the second lumen forming a junction such that the second end of the first lumen (e.g. a proximal end of a hollow needle) forms an opening in the wall of the second lumen at or near the constriction. Air velocity across the second end of the first lumen (e.g. a proximal end of a hollow needle) may be sufficiently high to create a low pressure at (e.g. in and/or around) the second end of the first lumen. Low pressure (e.g. pressure below atmospheric pressure) at the second end of the first lumen may create suction in the first lumen, which may cause one or more cores in the first lumen to be drawn towards and/or out of the second end of the first lumen. This process may occur alone or in combination with a force exerted by one or more (stacked) cores drawn into the first lumen, for example through a first end of the first lumen, such as when caused by movement of the first lumen into skin tissue, causing formation of new cores inside the first lumen.

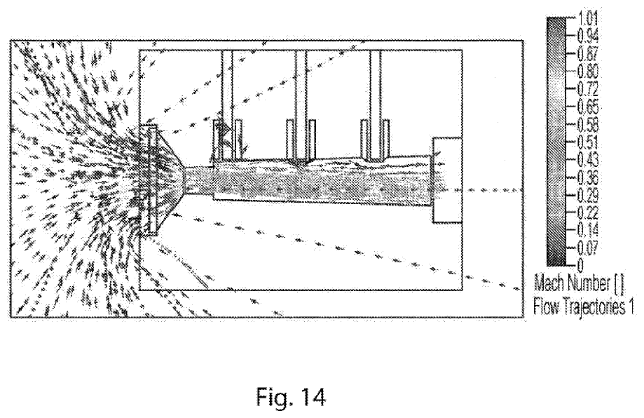

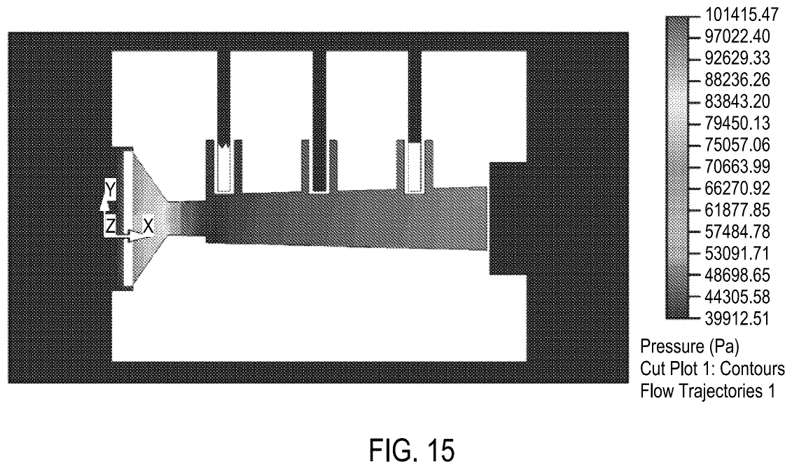

FIG. 14 and FIG. 15 show results of a computational fluid dynamics (CFD) simulation in an example channel (e.g. a second lumen) comprising a “steep” conical/frusto-conical inlet (convergent) and having a longer, “shallow” frusto-conical profile downstream from the inlet (divergent). The example model includes three additional channels (e.g. first lumens) that represent lumens including or connected to lumens of example needles. In this example simulation, coring needle lumens (e.g. first lumens) are blocked off to show flow effects as if cores were stacked up and blocking the needle lumens. Stacked cores may adhere to each other, which may require a very high velocity airflow, for example (near) supersonic flow (e.g. in cases in which gas is used), to “break off” each core from the stack of cores. Fluid flow rates and/or velocities may depend on a size of associated channels. Flow rates of a fluid (e.g. a gas such as air) may be adjusted such that the maximum Mach number in a channel is about 0.1, 0.15, 0.2, 0.25, 0.3, 0.35, 0.4, 0.45 0.5, 0.55, 0.6, 0.65, 0.7, 0.75, 0.8, 0.85, 0.9, 0.95, or 1.0. In some embodiments, the maximum Mach number in a channel is about 0.72. In some embodiments, flow in a channel may be supersonic (e.g. the maximum Mach number in a channel is about 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 or 2.0). FIG. 14 shows a cutaway section of the channels where arrows indicate direction of flow. Gray scale of arrows indicates flow velocity ranging from near zero outside the example channels to (near) supersonic flow (Mach 1) at or near the narrowest point of the example second channel. FIG. 14 shows a cutaway section of the channels where gray scale indicate fluid pressure. Reduced pressure can be observed in the divergent portion of the example channel, which may indicate a suction force on the first lumens.

As described further herein, an example fluid system including a first and second lumen as described above, may include or be connected to auxiliary technologies, such as one or more valves, pumps, filters, tissue traps, tubing, and tubing connectors. Fluid flow (e.g. air flow) through a lumen (e.g. a second lumen) may be controlled to be continuous or pulsed. In some embodiments, fluid flow is pulsed on/off. Pulsed flow may cause change in direction of force acting on a skin core, which may aid dislodgement of a skin core and/or transport in the fluid stream. In some embodiments, a fluid system (e.g. a suction system) may be directly connected to a lumen of one or more hollow needles (e.g. connected to one or more first lumens without a second lumen and without a cross-flow system as described above).

Other configurations and technologies may be used for fluid-based core clearing. In some embodiments, a stream of liquid may be employed instead of a stream of gas (e.g. an airstream) to remove one or more cores. In some embodiments, a closed-loop hydraulic system may be used to draw liquid through a lumen (e.g. a second lumen) to remove one or more cores.

In some embodiments, one or more cores 10-2000 may be removed from a lumen, for example a first lumen, by an internal removal tool such as a pushrod. In some embodiments, an internal tissue removal tool may be a piston or a pin that fits inside the lumen of a hollow needle, for example without creating a (partial) vacuum inside the lumen (e.g. the gap between the tissue removal tool and the wall of the lumen of the hollow needle may be large enough to allow the passage of air). In some embodiments, an internal removal tool may be a piston. A removal tool (e.g. a piston or pushrod) may not disrupt a structural integrity of a cored tissue portion. In some embodiments, an internal removal tool (e.g. a piston or pushrod) may push one or more cored tissue portions out of a lumen of a first lumen (e.g. a hollow needle) as a substantially intact, cored tissue portion, instead of as pieces of the cored tissue portion, which may be difficult to remove completely. Maintaining structural integrity of a cored tissue portion as a substantially intact tissue portion during a removal process may facilitate efficient and complete tissue removal from a hollow needle.

In some embodiments, a needle hub may not include a fluid-based core clearing system, for example as described above. In some embodiments, during repeated operation of one or more needles, cores in a lumen of a needle may be stacked and pushed out of a lumen (e.g. out of a proximal end of a needle lumen by positive displacement). Cores exiting from a proximal end of a needle lumen may be deposited into a space, such as a space comprising a receptacle, proximal to the needle.

In some embodiments, a system as described herein may include a rinsing system, for example including a saline flushing or rinsing system, such as to wash one or more needles between uses. In some embodiments, a rinsing system may include a sterile saline container that may receive one or more needles. In some embodiments, low pressure may be applied to the one or more needles drawing saline through the one or more needles, thus clearing any debris from one or more needle lumens.

In some embodiments, a lubricant may be used, for example a lubricant configured to enhance or otherwise facilitate core clearing. For example, a needle tip and/or air inlet may be sprayed with or dipped in a liquid (e.g. saline to aid tissue and fluid clearing).

In some embodiments, a lumen of a needle and/or a first lumen may be cylindrical. In some embodiments, a lumen of a needle and/or a first lumen may be frusto-conical, for example a proximal end of a lumen of a needle and/or a first lumen may have a larger diameter than a distal end for contacting skin (e.g. to improve tissue transit through the lumen).

Other tissue removal tools that may be used with the technologies described herein are described in PCT Application number PCT/US2017/02475, filed Mar. 29, 2017, the disclosure of which is incorporated herein by reference in its entirety for all purposes.

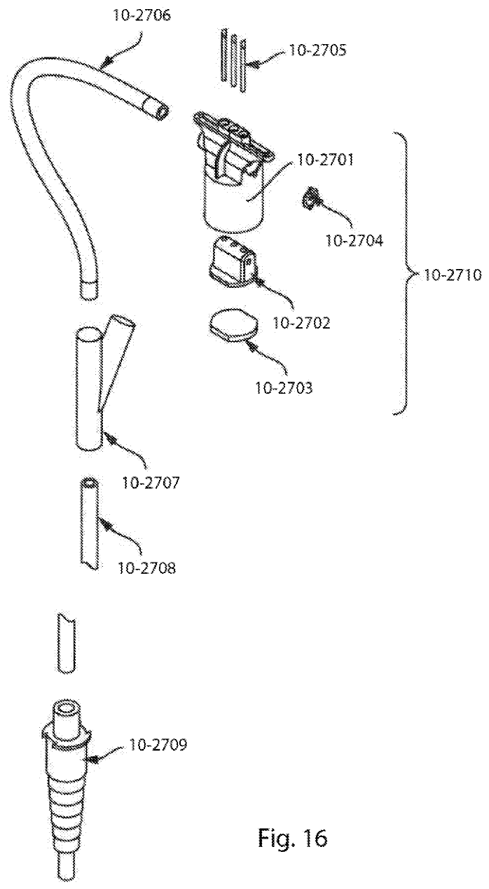

An example needle hub and core clearing system that may be used with the technologies described herein, for example apparatus 10-100, 10-200, or 10-400, is shown in FIG. 16. In some embodiments, a needle hub 10-2710 may include a needle hub body 10-2701 to hold, for example, three example needles 10-2705, and a needle hub insert 10-2702. In some embodiments, a needle (e.g. one or more of needles 10-2705) may be fully or partially inserted into one or more lumens of a needle hub (e.g. needle hub body 10-2701). A needle may be glued, welded, and/or press fit into a needle hub body. In some embodiments, a needle (e.g. one or more of needles 10-2705) may be attached to one or more lumens of a needle hub (e.g. needle hub body 10-2701), without being inserted, for example one or more needles may be attached externally to a needle hub body (e.g. needle hub body 10-2701). In some embodiments, an example needle hub 10-2710 may include a filter 10-2704, for example to remove impurities from ambient air. In some embodiments, an example needle hub 10-2710 may include a secondary insert 10-2703, such as a metal (e.g. steel) insert. In some embodiments, a secondary insert 10-2703 may be used, for example, to hold a needle hub insert 10-2702 in place. In some embodiments, a secondary insert 10-2703, such as a metal (e.g. steel) insert, may be used to verify that a needle hub 10-2710 is connected (e.g. securely connected) to one or more components of an apparatus as described herein, for example securely mounted to a z-actuator (e.g. via a needle hub mount). In some embodiments, an electrical signal or an RFID signal may be used to verify connection. In some embodiments, a secondary insert 10-2703 may include an RFID tag. In some embodiments, a needle hub, such as needle hub 10-2710, may be implemented as a disposable unit. In some embodiments, a needle hub, such as needle hub 10-2710, may be configured to include several components that are implemented as one or more disposable units (e.g. one or more needles and/or needle mounts). An example needle hub may include a tag, chip, and/or other identifier (e.g. an identifier mounted on and/or integrated into a secondary insert 10-2703). In some implementations, an identifier may be used to identify a specific needle hub, such as to monitor usage of a needle hub as described herein.

In some embodiments, an example needle hub 10-2710 and core clearing system may include a fluid conduit, such as first tubing 10-2706, which can be connected to needle hub 10-2710, for example at an end of a lumen of a needle hub body 10-2701 (e.g. a second end of a second lumen). In some embodiments, a fluid conduit, such as a first tubing 10-2706, may be connected to a connector, such as a Y-connector 10-2707 having a first end, a second end, and a third end. In some embodiments, a first tubing 10-2706 may be connected to a first end of a Y-connector 10-2707. In some embodiments, a Y-connector 10-2707 may include a second end, which may be connected to a fluid conduit (e.g. tubing) that may connect Y-connector 10-2707 to a spacer, such as a foot or frame of a vacuum spacer as described below. In some embodiments, a Y-connector 10-2707 may include a third end connected to a fluid conduit, such as second tubing 10-2708. In some embodiments, a second tubing 10-2708 may be connected to or include a connector, such as a stepped connector 10-2709. In some embodiments, a connector, such as stepped connector 10-2709, may connect a needle hub and/or core clearing system to a fluid system (e.g. a low pressure system such as a vacuum pump), to induce (partial) vacuum in a system as described below.

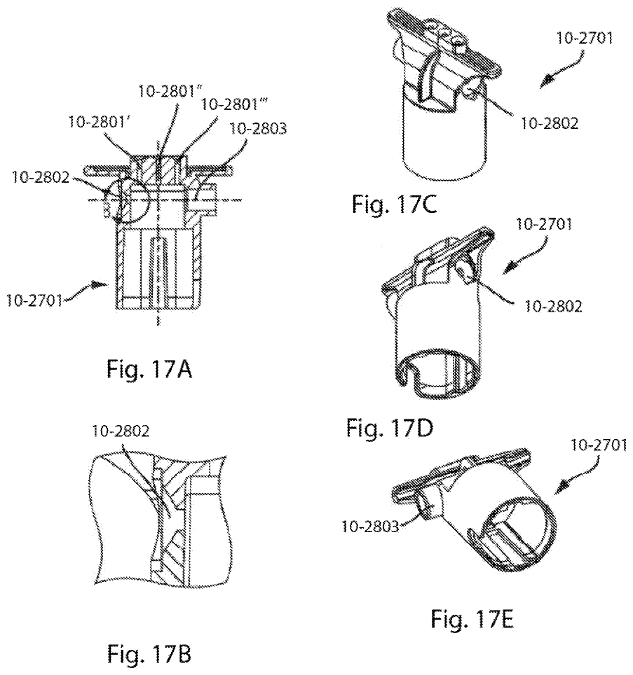

FIG. 17A shows a cross-sectional view of an example needle hub body 10-2701. In some embodiments, a needle hub body 10-2701 may include one or more first lumens, such as three first lumens 10-2801, or one or more parts thereof. In some embodiments, a needle may be fully or partially inserted in a first lumen (e.g. lumen 10-2801) of a needle hub body, such as needle hub body 10-2701. In some embodiments, a needle, for example a hollow needle having a lumen, may be attached to a needle hub body (e.g. needle hub body 10-2701), such that a first lumen of a needle hub body and a needle lumen are connected (e.g. end-to-end) and together form a first lumen of a needle hub (e.g. needle hub 10-2710), or a part thereof. In some embodiments, a needle hub body 10-2701, may include a fluid intake nozzle, such as nozzle 10-2802. In some embodiments, a fluid intake nozzle (e.g. nozzle 10-2802) of a needle hub body 10-2701, may constitute a part of a second lumen of a needle hub body, and/or it may be located at a first end of a second lumen of a needle hub body (see FIG. 17B). In some embodiments, a needle hub body, such as needle hub body 10-2701, may include a second lumen, or a part thereof (e.g. an upstream section 10-2803 of a second lumen of a needle hub body 10-2701). In some embodiments, a fluid intake nozzle and a second lumen of a needle hub body, for example an upstream section 10-2803 of a second lumen of a needle hub body 10-2701, may be part of a second lumen of a needle hub. FIGS. 17C-E show perspective views of an example needle hub body 10-2701.

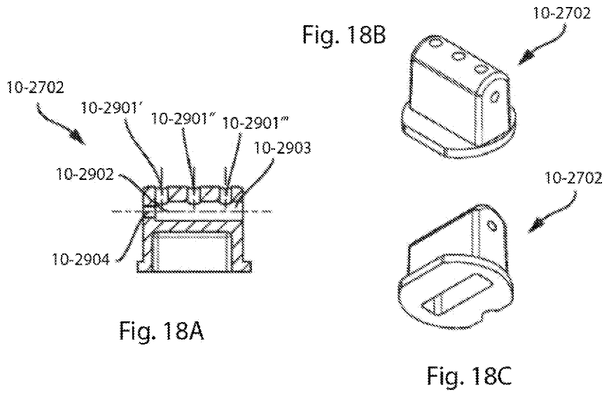

FIG. 18A shows a cross-sectional view of an example needle hub insert 10-2702. In some embodiments, a needle hub insert, for example needle hub insert 10-2702, may include one or more first lumens, for example three first lumens 10-2901, or one or more parts thereof. In some embodiments, a needle hub insert may be configured such that one or more first lumens of a needle hub insert 10-2702 line up with one or more first lumens of a needle hub body, for example first lumen 10-2801 of needle hub body 10-2701, when a needle hub insert is inserted in a needle hub body. In some embodiments, a lumen of a hollow needle, a first lumen of needle hub body (e.g. needle hub body 10-2701), and a first lumen of a needle hub insert (e.g. needle hub insert 10-2702), may be connected (e.g. end-to-end) and together form a first lumen of a needle hub, for example needle hub 10-2710, or a part thereof. For example (and referring to FIGS. 17A and 18A), lumen 10-2901′ may line up with lumen 10-2801′, lumen 10-2901″ may line up with lumen 10-2801″, and lumen 10-2901′″ may line up with lumen 10-2801′″ In some embodiments, a lumen of a hollow needle may be inserted in a needle hub body 10-2701 such that a lumen of a hollow needle, a lumen of a needle hub body 10-2701, and a first lumen of needle hub insert 10-2702 are connected (e.g. end-to-end) and together form a first lumen of a needle hub 10-2710, or a part thereof. In some embodiments, a needle hub insert 10-2702 may include a second lumen (e.g. second lumen 10-2902) having a first end and a second end. In some embodiments, a fluid intake nozzle, such as nozzle 10-2802, a second lumen (e.g. upstream section 10-2803) of a needle hub body 10-2701, and a second lumen 10-2902 of a needle hub insert 10-2702 may align and constitute a second lumen of a needle hub 10-2710 or may be part of a second lumen of a needle hub 10-2710. In some embodiments, a needle hub insert 10-2702 may be configured such that when needle hub insert 10-2702 is inserted into a needle hub body 10-2701, fluid entering a needle hub through an intake nozzle (e.g. nozzle 10-2802) of a needle hub body 10-2701 may subsequently enter a second lumen 10-2902 of a needle hub insert 10-2702 through an opening at a first end of a lumen of a needle hub insert 10-2702. Fluid may then traverse a second lumen 10-2902 of a needle hub insert 10-2702, and exit the second lumen 10-2902 of a needle hub insert 10-2702 through an opening at a second end 10-2903 of a second lumen 10-2902 of a needle hub insert 10-2702. Fluid exiting a second lumen 10-2902 of a needle hub insert 10-2702 through an opening 10-2903 at a second end of a second lumen 10-2902 of a needle hub insert 10-2702 may enter a second lumen of a needle hub body 10-2701 (e.g. an upstream section 10-2803 of a second lumen of a needle hub body 10-2701). In some embodiments, an opening 10-2903 at a second end of a second lumen 10-2902 of a needle hub insert 10-2702 has a larger cross sectional area than an opening 10-2904 at a first end of a second lumen 10-2902 of a needle hub insert. In some embodiments, a second lumen (e.g. upstream section 10-2803) of a needle hub body 10-2701, and a second lumen 10-2902 of a needle hub insert 10-2702 may constitute a second lumen of a needle hub 10-2710 or may be part of a second lumen of a needle hub 10-2710. FIG. 18B and FIG. 18C show perspective views of an example needle hub insert 10-2702.

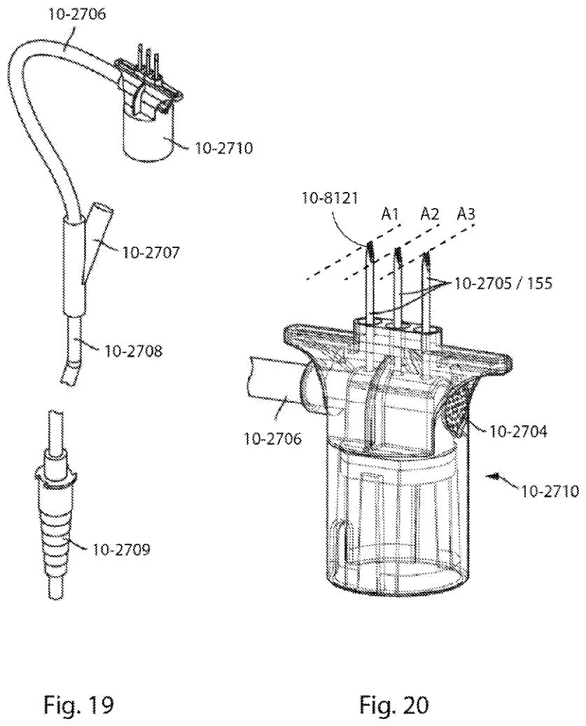

FIG. 19 shows an assembled example needle hub 10-2710 and core clearing system that may be used with the technologies described herein. FIG. 20 shows a semi-transparent view of an assembled example needle hub 10-2710 including three needles 10-2705.

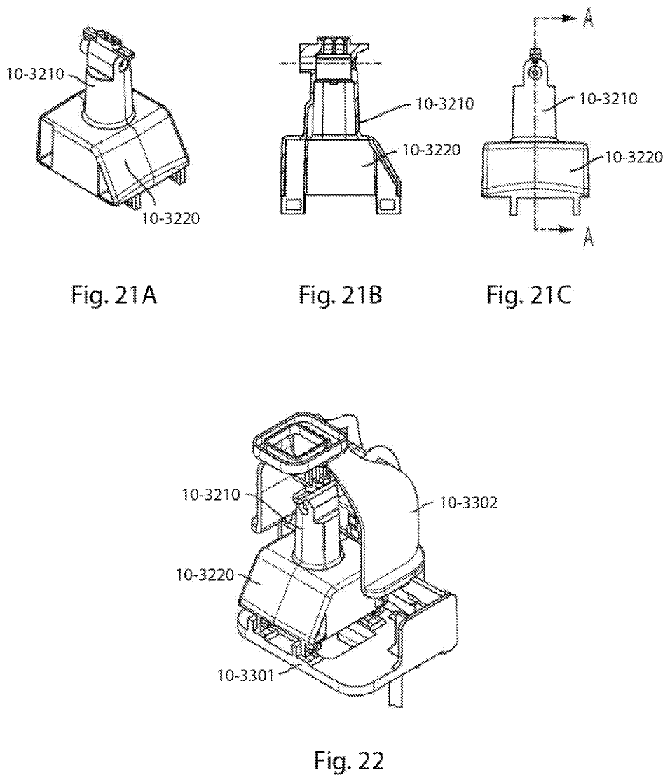

In some embodiments, a system as described herein may include technologies to prevent fluids or other substances from entering an apparatus (e.g. an apparatus 10-100, 10-200, or 10-400), such as to prevent fluids from entering a distal opening in a hand piece (e.g. hand piece 10-120, 10-220, or 10-420). In some embodiments, a needle hub may include or may be connected to a shield to prevent fluids or other substances from entering an apparatus, for example to prevent fluids from entering a distal opening in a hand piece (e.g. a hand piece 10-120, 10-220, or 10-420). FIGS. 21A-C show an example needle hub 10-3210, which may be substantially similar or the same as needle hub 10-2710, connected to example hub shield 10-3220. In some embodiments, as a needle hub, such as needle hub 10-3210, moves in the x-direction or y-direction (e.g. substantially parallel to a skin surface) and/or moves in the z-direction (e.g. substantially perpendicular to a skin surface), an example hub shield 10-3220 may move together with the needle hub 10-3210. In some embodiments, an example hub shield 10-3220 is sized such that a distal opening or distal end of a hand piece (e.g. a hand piece 10-120, 10-220, or 10-420) is covered by at least a portion of a hub shield 10-3220 and/or needle hub 10-3210. FIG. 22 shows an example needle hub 10-3310 and hub shield 10-3220 moveably mounted on an example hand piece distal end component 10-3301 and an example spacer 10-3302. Example spacers, such as spacer 10-3302, are further described below (e.g. spacers 10-4000 or 10-4100). In some embodiments, example needle hub 10-3310 and hub shield 10-3220, example hand piece distal end component 10-3301 and/or example spacer 10-3302 may be reusable. In some embodiments, example needle hub 10-3310 and hub shield 10-3220, example hand piece distal end component 10-3301 and/or example spacer 10-3302 may be disposable. In some embodiments, example hub shield 10-3220 may be releasably connected to hand piece distal end component 10-3301. In some embodiments, for example where needle hub, hub shield, and or hand piece distal end component are disposable, example hub shield 10-3220 may be connected to hand piece distal end component 10-3301 during storage and/or transport (e.g. through a openable locking mechanism, e.g. a hooking mechanism), but may be released from hand piece distal end component 10-3301 after hand piece distal end component 10-3301 is connected to a hand piece, such as hand piece 10-220. Other example embodiments are discussed below.

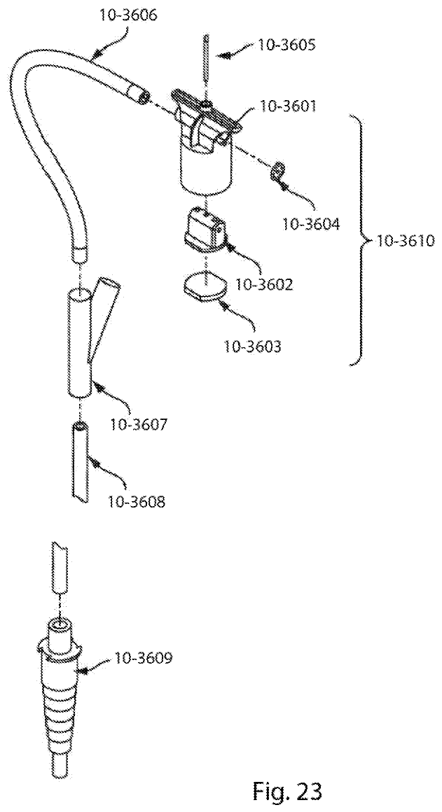

FIG. 23 shows an example needle hub and core clearing system substantially as described above (e.g. as described for the embodiment in FIG. 16), configured for a single needle, such as needle 10-3605. In some embodiments, a needle hub 10-3610 may include a needle hub body 10-3601 to hold, for example an example needle 10-3605, and a needle hub insert 10-3602. Needle hub insert 10-3602 may be substantially similar or the same as needle hub insert 10-2702. In some embodiments, a needle, such as a needle 10-3605, may be fully or partially inserted into one or more lumens of a needle hub, such as needle hub body 10-3601. A needle may be glued, welded, and/or press fit into a needle hub body. In some embodiments, a needle, such as needle 10-3605, may be attached to one or more lumens of a needle hub (e.g. needle hub body 10-3601), without being inserted (e.g. a needle may be attached externally to a needle hub body, such as needle hub body 10-3601). In some embodiments, an example needle hub 10-3610 may include a filter 10-3604, such as a filter configured to remove impurities from ambient air. In some embodiments, an example needle hub 10-3610 may include a secondary insert 10-3603, such as a metal (e.g. steel) insert. In some embodiments, a secondary insert 10-3603 may be used, such as to hold a needle hub insert 10-3602 in place. In some embodiments, a secondary insert 10-3603, such as a metal (e.g. steel) insert, may be used to verify that a needle hub 10-3610 is connected (e.g. securely connected) to one or more components of an apparatus as described herein (e.g. securely mounted to a z-actuator) such as via a needle hub mount. In some embodiments, an electrical signal or an RFID signal may be used to verify connection. In some embodiments, a secondary insert 10-3603 may include an RFID tag. In some embodiments, a needle hub, such as needle hub 10-3610, may be implemented as a disposable unit. In some embodiments, a needle hub, such as needle hub 10-3610, may be configured to include several components that are implemented as one or more disposable units (e.g. and/or as a needle mount). An example needle hub may include a tag, chip, and/or other identifier, for example mounted on and/or integrated into a secondary insert 10-3603. In some implementations, an identifier may be used to identify a specific needle hub, such as to monitor usage of a needle hub as described below.

In some embodiments, an example needle hub 10-3610 and core clearing system may include a fluid conduit, for example first tubing 10-3606 (e.g. connected to needle hub 10-3610, such as at an end of a lumen of a needle hub body 10-3601, for example a second end of a second lumen). In some embodiments, a fluid conduit such as a first tubing 10-3606, may be connected to a connector, such as a Y-connector 10-3607 having a first end, a second end, and a third end. In some embodiments, a first tubing 10-3606 may be connected to a first end of a Y-connector 10-3607. In some embodiments, a Y-connector 10-3607 may include a second end, which may be connected to a fluid conduit (e.g. tubing) that may connect Y-connector 10-3607 to a spacer, such as a foot or frame of a vacuum spacer as described below. In some embodiments, a Y-connector 10-3607 may include a third end connected to a fluid conduit, such as second tubing 10-3608. In some embodiments, a second tubing 10-3608 may be connected to or include a connector, such as a stepped connector 10-3609. In some embodiments, a connector, such as stepped connector 10-3609, may connect a needle hub and/or core clearing system to a fluid system, such as a low pressure system, for example a vacuum pump, to induce (partial) vacuum in a system as described below.

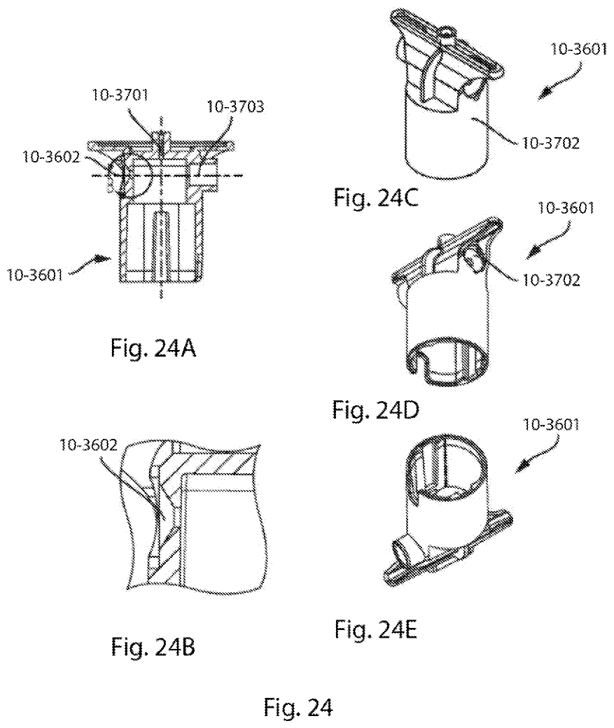

An example needle hub body 10-3601 to be used with an example system as shown in FIG. 23 is shown in FIGS. 24A-E. In some embodiments, a needle hub insert as shown, for example inserts 10-3602 or 10-2702 in FIGS. 18A-C may be used in a single needle system as shown in FIG. 23. In some embodiments, a needle hub insert with a single first lumen may be used in a single needle system as shown in FIG. 23. FIG. 24A shows a cross-sectional view of an example needle hub body 10-3601. In some embodiments, a needle hub body 10-3601 may include a first lumen, such as first lumen 10-3701, or one or more parts thereof. In some embodiments, a needle may be fully or partially inserted in a first lumen of a needle hub body, such as needle hub body 10-3601. In some embodiments, a needle, such as a hollow needle having a lumen, may be attached to a needle hub body, for example needle hub body 10-3601, such that a first lumen 10-3701 of a needle hub body and a needle lumen are connected (e.g. end-to-end) and together form a first lumen of a needle hub, such as needle hub 10-3610, or a part thereof. In some embodiments, a lumen of a hollow needle, a first lumen of a needle hub body (e.g. needle hub body 10-3601), and a first lumen of a needle hub insert (e.g. needle hub insert 10-3603 or 10-2702), may be connected (e.g. end-to-end) and together form a first lumen of a needle hub, such as needle hub 10-3610, or a part thereof. In some embodiments, a lumen of a hollow needle, a first lumen of a needle hub body (e.g. needle hub body 10-3601), and a first lumen 10-2901 of needle hub insert 10-2702, may be connected (e.g. end-to-end) and together form a first lumen of a needle hub, such as needle hub 10-3610, or a part thereof. In some embodiments, a needle hub body 10-3601 may include a fluid intake nozzle, such as nozzle 10-3702. In some embodiments, a fluid intake nozzle, such as nozzle 10-3702, of a needle hub body 10-3601, may constitute a part of a second lumen of a needle hub body, and/or may be located at a first end of a second lumen of a needle hub (e.g. as shown in FIG. 24B). In some embodiments, a needle hub body, such as needle hub body 10-3601, may include a second lumen, or a part thereof, for example an upstream section 10-3702 of a second lumen of a needle hub body 10-3601. In some embodiments, a fluid intake nozzle, such as nozzle 10-3702, and a second lumen of a needle hub body, such as an upstream section 10-3703 of a second lumen of a needle hub body 10-3601, may be part of a second lumen of a needle hub. FIGS. 24C-E show perspective views of an example needle hub body 10-3601.

### Consumable Detection—Needle Hub

A needle hub may be configured and/or implemented as a single-use item or a reusable item. In some embodiments, a reusable needle hub may be sterilizable (e.g. re-sterilizable) and/or autoclavable (e.g. may be constructed from heat resistant materials).

In some embodiments, a needle hub as described herein (e.g. needle hub 10-2710 or needle hub 10-3610) may include a tag to identify a needle hub. In some embodiments, a tag may be or include an integrated circuit (IC) chip that may be read-only (e.g. include read-only memory). In some embodiments, a tag may be or include a chip that may be a read-and-write chip. In some embodiments, a tag may be or include a chip that is operable to exchange data with a reader using, for example, RF signals, and may include a built-in antenna and an integrated circuit, for example a tag may be or include an RFID tag. In some embodiments, a tag may be or include an RFID chip mounted on or integrated into a needle hub, for example in or on a secondary insert (e.g. secondary insert 10-2703 or 10-3603) of a needle hub (e.g. needle hub 10-2710 or needle hub 10-3610).

In some implementations, an identifier may be used to identify a specific needle hub, for example to monitor usage of a needle hub as described below.

### Spacer

The technologies described herein may include a system and/or apparatus that includes a spacer. In some embodiments, a spacer may be part of or connected to an apparatus as described herein (e.g. apparatus 10-100, 10-200, or 10-400), for example a spacer may be part of or attached to a hand piece (e.g. hand piece 10-120, 10-220 or 10-420), for example to a hand piece shell, of an example apparatus. In some embodiments, a spacer may be used to maintain a constant distance between a base position (e.g. retracted position) of a needle and a surface of skin to be treated. In some embodiments a spacer may be adjustable or moveable, such as to adjust the distance between a base position of a needle (and/or a distance between a z-actuator) and a surface of skin to be treated. In some embodiments, a distance between a base position of a needle (and/or a distance between a z-actuator) and a surface of skin to be treated may be adjusted during a coring procedure. In some embodiments, a distance between a base position of a needle (and/or a distance between a z-actuator) and a surface of skin to be treated may be adjusted prior to a coring procedure and may remain constant during a coring procedure.

In some embodiments, a spacer may be or include a one or more devices to maintain a distance and/or position (e.g. a constant distance and/or position) of an apparatus relative to a skin surface to be treated during a coring procedure. In some embodiments, a spacer may be or include a one or more devices configured to maintain and/or increase tension in a skin tissue to be treated (e.g. during treatment) compared to skin not being treated and/or not contacted by an apparatus described herein. In some embodiments, one or more devices configured to maintain a distance and/or position relative to tissue are different from one or more devices configured to maintain and/or increase tension in a skin tissue. In some embodiments, one or more devices configured to maintain a distance and/or position are the same as one or more devices configured to maintain and/or increase tension in a skin tissue. In some embodiments, one or more devices configured to maintain a distance and/or position and/or one or more devices configured to maintain and/or increase tension in a skin tissue may include hooks and/or barbs, and/or they may include one or more tissue fixation implements including frames, pins, rollers, forceps, grippers, hooks, needles, barbs, and/or adhesives.

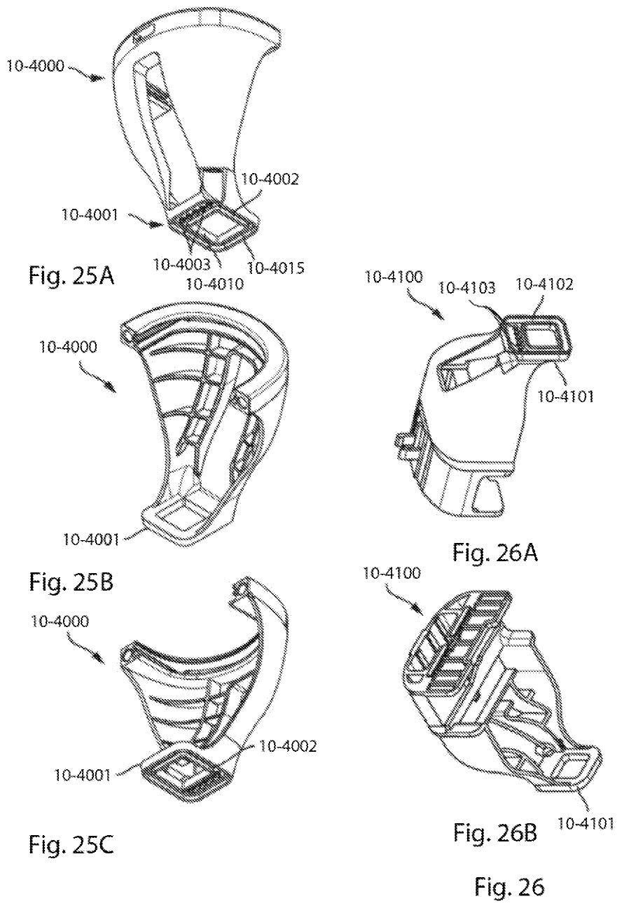

In some embodiments, a spacer may be or include a vacuum spacer. An example vacuum spacer 10-4000 is shown in FIGS. 25A-C. An example vacuum spacer may include a frame 10-4001 to contact a surface of a skin tissue to be treated. In some embodiments, a frame 10-4001 of a spacer 10-4000 may be configured such that the frame forms a border around an area of skin to be treated, for example to be cored by one or more coring needles. An example frame of a spacer may include a base, an inner wall 10-4010, and an outer wall 10-4015, wherein the base, inner wall, and outer wall form an open channel in the frame. An example channel 10-4002 may be configured such that when a frame is placed on a surface of skin, the surface of the skin, the base, the inner wall 10-4010, and outer wall 10-4015 form a lumen, for example a frame lumen. In some embodiments, a frame 10-4001 may include one or more protrusions, such as one or more protrusions 10-4003, for example to reduce an amount of skin tissue drawn into a channel 10-4002.

FIGS. 26A and 26B show another, similar, example spacer 10-4100 with (vacuum) frame 10-4101, example frame channel 10-4102, and example protrusions 10-4103. Example spacer 10-4100 may be substantially similar or the same as spacer 10-3302 shown in FIG. 22.

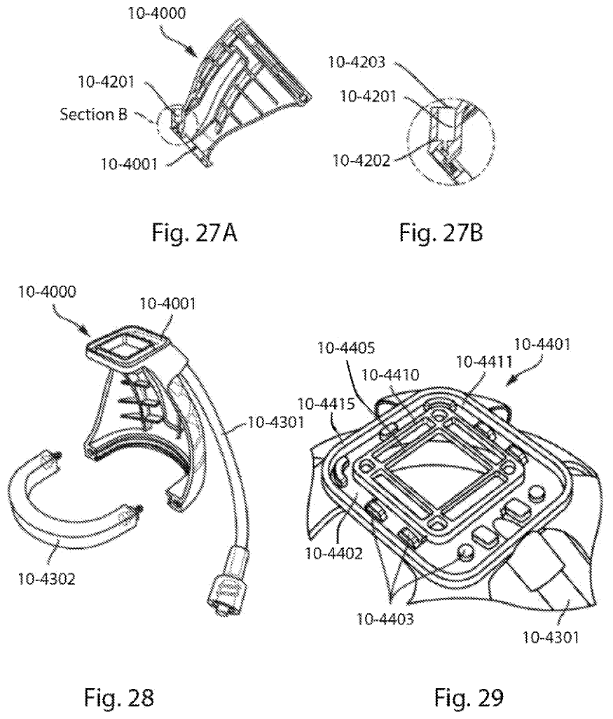

In some embodiments, a frame, such as frame 10-4001 or 10-4101, may be connected to a fluid conduit such that when low pressure (e.g. below atmospheric pressure) or (partial) vacuum is applied to the fluid conduit, low pressure or (partial) vacuum is established in the frame lumen (e.g. frame channel 10-4002 or 10-4102). FIGS. 27A and 27B show a section of an example vacuum spacer frame (e.g. frame 10-4001) including a connection lumen 10-4201 having a first end 10-4202 and a second end 10-4203. In some embodiments, a first end 10-4202 of a connection lumen 10-4201 may form an opening in a frame lumen, such as channel 10-4002. In some embodiments, a second end 10-4203 of a connection lumen 10-4201 may contact an end of a lumen of a fluid conduit. In some embodiments, a fluid conduit may be connected to a Y-connector 10-2707 as shown in FIG. 16 (e.g. a second end of a Y-connector) and/or a low pressure source, such as a vacuum pump. Low pressure or (partial) vacuum in a frame lumen may cause skin tissue to be drawn towards (e.g. sucked into) the channel.

In some embodiments, applying low pressure (e.g. below atmospheric pressure) or (partial) vacuum to a channel of a vacuum spacer frame (e.g. channel 10-4002 or 10-4102) may cause a suction force to be exerted on skin tissue contacting the frame. This suction force may cause an increase in tension in an area of skin near (e.g. surrounded by) and/or in contact with a vacuum spacer frame. Without intending to be bound by theory, increased tension in skin tissue surrounded by a vacuum spacer frame under low pressure or (partial) vacuum may cause stabilization of a plane of the skin surface such that when surface penetration by a coring needle begins, movement of skin in contact with the needle in direction of needle movement during coring (“tenting”) is reduced compared to movement of skin during a similar procedure without application of a vacuum spacer frame. A coring needle may penetrate a dermis at a lower velocity and/or force than would be required in a similar procedure without application of a vacuum spacer frame to a skin surface. In some example embodiments, application of a vacuum spacer frame may yield more consistent/reproducible depth of penetration of a needle, for example in relation to a skin surface and/or a vacuum spacer frame, compared to a similar procedure without application of a vacuum spacer frame (e.g. due to reduced movement of skin). In some example embodiments, application of a vacuum spacer frame may lead to a lower depth of penetration of a needle, for example in relation to a skin surface and/or a vacuum spacer frame, required to achieve a similar effect compared to a similar procedure without application of a vacuum spacer frame (e.g. due to reduced movement of skin or compression of one or more skin layers). Use of a vacuum spacer frame may reduce trauma (reduce down time), enhance safety, and/or reduce chances of over-penetration. In some embodiments, a low pressure or (partial) vacuum generated in a vacuum frame may enable a user to pull skin tissue connected to the vacuum frame away from anatomical structures beneath the skin, for example reducing the potential for the needle to contact undesired underlying structures during actuation. In an example procedure without a vacuum spacer frame, a coring needle may push skin away in direction of needle tip movement as the needle is penetrating skin, which may necessitate a deeper penetration by a needle to reach the patient's lower dermis and adjacent fat layer, such as to remove a full thickness core.

FIG. 28 shows an example vacuum spacer (e.g. spacer 10-4000), an example fluid conduit 10-4301 connected to a frame 10-4001 of the vacuum spacer 10-4000, and a connection frame 10-4302 to connect a vacuum spacer (e.g. vacuum spacer 10-4000) to, for example, a hand piece (e.g. hand piece 10-120, 10-220, or 10-420), and/or a hand piece shell (e.g. hand piece shell 10-121, 10-221, or 10-421), of a coring apparatus (e.g. apparatus 10-100, 10-200, or 10-400). FIG. 29 shows an example vacuum spacer frame 10-4401 (substantially similar to frame 10-4001 and frame 10-4101) which is configured to draw skin within the frame taught, such as to stabilize skin during coring. In some embodiments, a vacuum spacer frame (e.g. frame 10-4401) may include a sub-frame (e.g. sub-frame 10-4405), such as to aid positioning of a frame and/or to provide tissue stabilization.

In some embodiments, a channel in a vacuum spacer frame (e.g. frame 10-4401) may include one or more protrusions (e.g. protrusions 10-4403), for example one or more structures protruding from a base (e.g. base 10-4411) of a channel (e.g. channel 10-4402) in a vacuum spacer frame, such as to ensure even suction pressure throughout a frame lumen, such as is shown in FIG. 29. When low pressure or (partial) vacuum is applied to a lumen formed by a channel in a vacuum spacer frame and a skin surface (e.g. a frame lumen), skin tissue may be drawn toward the base of the channel. Skin tissue may block a first end of a connection lumen that may form an opening in a frame lumen, blocking fluid communication between the frame lumen and a fluid conduit (e.g. conduit 10-4301) that provides low pressure or (partial) vacuum, potentially disrupting a low-pressure connection between a vacuum spacer frame and a skin surface. One or more structures protruding from a base of a channel in a vacuum spacer frame may be configured to prevent blocking of a first end of a connection lumen by skin tissue. In some embodiments, a channel in a vacuum spacer frame includes one or more indentations, such as one or more cavities extending into a base of a channel in the vacuum spacer frame (e.g. to ensure even suction pressure throughout a frame lumen). In some embodiments, one or more cavities extending into a base of a channel (e.g. base 10-4411) in a vacuum spacer frame may be configured to prevent blocking of a first end of a connection lumen by skin tissue. In some embodiments, a channel in a vacuum spacer frame may include one or more protrusions (e.g. protrusions 10-4003, 10-4103, or 10-4403) and one or more indentations, such as to ensure even suction pressure throughout a frame lumen. In some embodiments, frame channel configurations, protrusion configurations, and/or indentation configurations may be chosen and/or modified depending on tissue type and/or location to be treated. Without intending to be bound by theory, the softer or laxer a skin tissue, the closer and/or the larger protrusion may be to prevent or impede skin tissue from entering space between a protrusion and a wall (e.g. outer wall 10-4415 and inner wall 10-4410) and/or another protrusion.

In some embodiments, a channel (e.g. channel 10-4002, 10-4102, or 10-4402) of a vacuum spacer frame may have a width of about 2.5 mm (e.g. a minimum distance of 2.5 mm between an inner wall, such as inner wall 10-4010 or 10-4410, and an outer wall, such as outer wall 10-4015 or 10-4415, of a frame, such as frame 10-4001, 10-4101, or 10-4401). In some embodiments, a channel of a vacuum spacer frame may have a depth (e.g. an average depth) of about 2 mm (e.g. an average distance between a base of a frame and a flat surface opposite the base and substantially in contact with an outer wall of the frame). A channel of a vacuum spacer frame may have any size, for example a size depending on tissue to be stabilized and/or a size configured to improve access to complex anatomical areas. In some embodiments, a channel of a vacuum spacer frame may have a width (e.g. a minimum distance between an inner wall and outer wall of a frame) of about 0.5 mm, 1 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, a channel of a vacuum spacer frame may have a width (e.g. a minimum distance between an inner wall and outer wall of a frame) of between 0 mm and 100 mm, between 10 mm and 90 mm, between 20 mm and 80 mm, or between 30 mm and 70 mm.

In some embodiments, a channel (e.g. channel 10-4002, 10-4102 or 10-4402) of a vacuum spacer frame may have a depth (e.g. an average distance between a base of a frame, such as base 10-4411 of frame 10-4401, and a flat surface opposite the base and substantially in contact with an outer wall, such as outer wall 10-4415, of the frame) of about 0.5 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, a channel of a vacuum spacer frame may have a depth (e.g. an average distance between a base of a frame, such as base 10-4411 of frame 10-4401, and a flat surface opposite the base and substantially in contact with an outer wall of the frame) of between 0 mm and 100 mm, between 10 mm and 90 mm, between 20 mm and 80 mm, or between 30 mm and 70 mm.

Size and/or shape of a frame of a vacuum spacer may depend on the location on a body of a subject on which an apparatus may be used. Multiple variations may be employed. In some embodiments, an area of skin enclosed or surrounded by a spacer frame, for example surrounded by an inner wall (e.g. wall 10-4410 of frame 10-4401), may have any area, such as an area of about 0.2 cm², 0.4 cm², 0.6 cm², 0.8 cm², 1.0 cm², 1.2 cm², 1.4 cm², 1.6 cm², 1.8 cm², 2.0 cm², 2.2 cm², 2.4 cm², 2.6 cm², 2.8 cm², 3.0 cm², 3.5 cm², 4.0 cm², 4.5 cm², 5.0 cm², 5.5 cm², 6.0 cm², 6.5 cm², 7.0 cm², 7.5 cm², 8.0 cm², 8.5 cm², 9.0 cm², 9.5 cm², 10 cm², 15 cm², or 20 cm². In some embodiments, a frame of a spacer, such as a vacuum spacer, may include a sub-frame or other structure, for example a structure positioned between inner walls of a frame, for example as shown in FIG. 29. In some embodiments, a sub-frame (e.g. a grid) or other structure may be used to further stabilize tissue or for alignment of an apparatus.

In some embodiments, a frame of a vacuum spacer may include non-contiguous vacuum channel sections, for example two longer channels on opposite sides of a frame (e.g. in a rectangular frame), or non-orthogonal sections. Frame elements may be straight and/or curved (e.g. include both straight and curved portions), and/or the frame elements may be orthogonal to each other and/or at different angles to each other. Inner and outer channel walls may have the same height. In some embodiments, an inner wall may have a greater height than the height of an outer wall. Varying configurations may increase or decrease an amount of stretch induced by a frame of a vacuum spacer.

The pressure in a system, for example the pressure in a frame lumen of a vacuum spacer, may range from approximately full vacuum (0 kPa) to approximately 50 kPa, for example a pressure may be between about 0 kPa and ambient atmospheric pressure, 0 kPa and 100 kPa, 5 kPa and 90 kPa, 10 kPa and 80 kPa, 15 kPa and 70 kPa, 20 kPa and 65 kPa, 25 kPa and 60 kPa, or 30 kPa and 50 kPa. In some embodiments, pressure may be kept constant during a coring procedure, or the pressure may be adjusted. In some embodiments, pressure may be monitored, for example by measuring fluid flow rate and/or pressure. Tissue properties may be monitored, for example to monitor underlying tissue behavior. In some embodiments, a frame may include or be connected to one, two, or more sensors, such as pressure sensors, electrical sensors, optical sensors, and/or cameras.

In some embodiments, a spacer may include a pressure switch, such as a switch configured to control actuation of a z-actuator. Once a vacuum spacer of an apparatus (e.g. apparatus 10-100, 10-200, or 10-400) is connected to a skin surface of a subject, and low pressure or (partial) vacuum is applied to a frame of a vacuum space, a user may move (e.g. gently pull up) the apparatus away from the skin surface, for example in a direction away from and substantially perpendicular to the skin surface. During movement of an apparatus, contact between apparatus and skin may be maintained. During movement, skin connected to an apparatus may be lifted away and/or detached from underlying tissue. During a coring procedure, a needle entering a skin tissue that has been lifted as described may be prevented from contacting tissue below a dermal layer and/or subcutaneous fat layer, even if a needle may over-penetrate a skin layer, for example due to an improper coring depth setting for a z-actuator.

In some embodiments, a system and/or apparatus as described herein may include a pressure switch, for example to prevent a z-actuator from moving unless an apparatus attached to skin tissue has been moved (e.g. pulled up) as described above. In some embodiments, a digital control system used with systems and apparatuses as described herein may include a pressure switch that may be connected to a sensor to detect a position of an apparatus relative to a skin surface and/or tissue underlying skin. In some embodiments, when a frame is placed on a skin surface and a low pressure or (partial) vacuum is applied to the frame, a switch is in a “no-go” position. After an apparatus and/or frame, while the frame is in contact with the skin surface after a low pressure or (partial) vacuum is applied to the frame, is moved in a direction that is substantially perpendicular to and away from the surface of the skin, the switch is in (e.g. transitions into) a “go” position. A switch may be actuated (e.g. mechanically or electrically) through a signal from a sensor that continuously senses a contact pressure between a frame and skin in contact with the frame or skin or skin tissue immediately adjacent thereto (e.g. skin tissue less than 20 mm, 15 mm, 10 mm, or 5 mm apart from an outer wall of a frame). Moving (e.g. pulling up) an apparatus may reduce contact pressure. In some embodiments, when contact pressure is reduced below a threshold, a switch may move from a “no-go” to a “go” position. When a switch is in the no-go position, a needle hub and/or z-actuator is prevented (e.g. by a digital control system) from moving along a z-axis in a direction substantially perpendicular to a surface of the skin tissue and substantially parallel to a longitudinal axis of the at least one hollow needle. When a switch is in the go position, a needle hub and/or z-actuator is moveable along the z-axis.

A spacer, such as a vacuum spacer as described herein, may provide an enhancement of safety wherein untargeted deeper tissues remain out of reach of a needle tip. Should a user push an apparatus down (distally) toward a skin surface, a switch may be moved to (or may remain in) a “no go” or treatment inhibited position. If a user pulls up and away from a skin surface, a pushrod may extend toward the distal end of a spacer frame moving a switch to the “go” or treatment enabled position. An apparatus with a pressure switch may be used as a technique training aid and/or it may be used to teach the proper “pull up” technique.

In some embodiments, a spacer, such as a vacuum spacer, as described herein, may include one or more tissue fixation implements including hooks, needles, barbs, and/or adhesives, for example to temporarily attach skin tissue to a frame. In some embodiments, an apparatus as described herein (e.g. apparatus 10-100, 10-200, or 10-400) may be used for treatment of facial tissue in combination with other implements. In some embodiments, application of a tongue depressor may help induce tension in tissue, for example in the face and/or neck, which may be beneficial for a coring procedure. After treatment, a cold (e.g. frozen) towel may be applied to cored tissue, such as to improve healing and/or increase tension in skin (e.g. to improve further treatment).

### Accessories

### Vacuum Pump System

In some embodiments, a system and/or apparatus (e.g. apparatus 10-100, 10-200, or 10-400) as described herein may include one or more low pressure and/or (partial) vacuum generation systems, such as to provide low pressure or (partial) vacuum for core clearing from a needle hub and/or to provide low pressure or (partial) vacuum (e.g. suction) to a spacer frame and/or needle hub. In some embodiments, a system or apparatus as described herein may include a low pressure or (partial) vacuum system that employs a single pump, a regulator, a control valve, and/or an inlet filter. In some embodiments, a system or apparatus as described herein may include a low pressure or (partial) vacuum system that employs multiple (e.g. two, three, or four) pumps, regulators, control valves, and/or inlet filters.

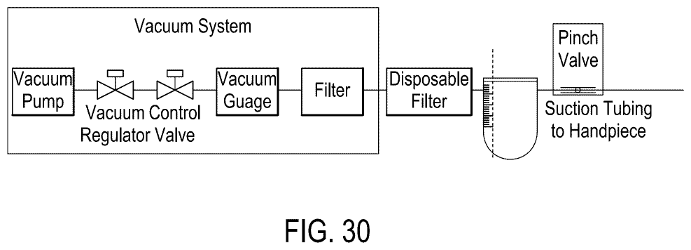

In some embodiments, a pressure conduit (e.g. tubing) connecting an element of a low pressure or (partial) vacuum system (e.g. a pump) to an apparatus (e.g. a hand piece) may be disposable. In some embodiments, a low pressure or (partial) vacuum system may include one or more filters, such as an air inlet filter, to remove debris from ambient air while air is drawn into a system or apparatus, and/or it can include one or more filters positioned between a needle hub and a pump, such as to protect a pump from debris and/or contamination. In some embodiments, a low pressure or (partial) vacuum system may include one or more traps (e.g. a fluid trap and/or a skin core collection trap) positioned upstream of a needle hub. In some embodiments, a low pressure or (partial) vacuum system may include a connection conduit to connect a vacuum spacer (e.g. a spacer frame) and a needle hub. In some embodiments, a valve, such as an electronic pinch valve, may be used to control flow rate and/or pressure (e.g. suction) in a conduit, such as by collapsing one or more sections of tubing. Pressure may also be controlled using other types of valves. In some embodiments, one or more solenoid valves may be used for pressure control. In some embodiments, a low pressure or (partial) vacuum system may include an internal pressure accumulator to improve system response. A diagram of an example low pressure or (partial) vacuum system is shown in FIG. 30.

In some embodiments, pressure in a low pressure or (partial) vacuum system may be controlled through a manual valve including a vent or opening (e.g. a vent or opening in a conduit), to ambient air. An example vent or opening to ambient air may be closed when low pressure or (partial) vacuum, for example suction, is desired. An example vent or opening may be closed by a valve or by covering a vent or opening by a finger of a user.

In some embodiments, a low pressure or (partial) vacuum system may include one or more pressure gauges and/or one or more flow meters to monitor pressure in a low pressure or (partial) vacuum system or components thereof (e.g. to measure pressure continuously or intermittently). In some embodiments, a low pressure or (partial) vacuum system may include or be connected to a digital processing unit for active control and monitoring of suction function and/or performance in a subsystem for core clearing from a needle hub. In some embodiments, a low pressure or (partial) vacuum system may continuously adjust suction force for each function.

### Positioning and Alignment

In some embodiments, a system or apparatus (e.g. a hand piece, e.g. hand piece 10-120, 10-220, or 10-420) as described herein may include a translation mechanism configured to drive an apparatus across the skin (e.g. perform x- and y-translation). In some embodiments, a translation mechanism may include driving wheels and/or rods. In some embodiments, a translation mechanism may permit automatic or manual translation of an apparatus across the skin. Translating components (e.g. wheels) may be disposed in or on the apparatus or may be disposed external to the apparatus, for example disposed in or on a hand piece and/or disposed at a location external to the hand piece. In some embodiments, a translating mechanism may be activated by an activator, such as a button, key, toggle, switch, screw, cursor, dial, spin-wheel, and/or other component, and/or the translating mechanism may be digitally controlled using a digital processing unit and a user interface.

In some embodiments, a system or apparatus (e.g. a hand piece, e.g. hand piece 10-120, 10-220, or 10-420) as described herein may include a position detection device or system, such as an optical tracking system. In some embodiments, a position detection system may be or include a camera, an infrared sensor, a photodiode, an LED, and/or a detector, and may assist in tracking movement of an apparatus in relation to a subject or a treatment area. An optical tracking mechanism may facilitate placement of a hollow needle on a skin surface in the instance of manual translation of the apparatus across the skin. In some embodiments, control electronics for a position detection mechanism may be disposed within the apparatus or external to the apparatus, for example when integrated into a digital processing unit as described herein. In some embodiments, a position detection mechanism may monitor a distance between a previous needle insertion and the current apparatus position, and the detection mechanism can send a signal to the control electronics to actuate the skin penetration mechanism when the apparatus has reached a desired position (e.g. a position at a defined distance from the position where the needles were last inserted). Desired distances and/or positions may be controlled at a user interface in communication with a digital processing unit.

In some embodiments, a system or apparatus as described herein may also include a guide or template to facilitate the positioning (e.g. alignment) of an apparatus and/or of a needle hub and/or of one or more hollow needles of the apparatus. In some embodiments, a guide or template may include one or more holes or openings that provide a pre-set array pattern (e.g. as described further herein) for one or more hollow needles of an apparatus to follow. A guide or template may be used alone or in combination with a position detection mechanism. In some embodiments, a hollow needle may be translated by x- and/or y-actuators to move across a guide or template and follow an array pattern set by the guide or the template to remove skin tissue portions at the holes or openings in the guide or template.

In some embodiments, a system or apparatus (e.g. apparatus 10-100, 10-200, or 10-400) may be positioned and/or aligned using an alignment frame. In some embodiments, a distal part of an apparatus, such as a spacer frame (e.g. a frame of a vacuum spacer as described above, such as frame 10-4001, 10-4101, or 10-4401), may be placed in, on, and/or around an alignment frame, for example along markings on an alignment frame (e.g. visual markers, protrusions, or magnets), such as to align an apparatus on a surface to be treated. In some embodiments, markers on an alignment frame may include protrusions or indentations in the alignment frame. In some embodiments, an alignment frame may be connected to a low pressure or (partial) vacuum system, for example as described herein, such as to stabilize underlying tissue as described herein with regards to a vacuum spacer frame.

After completion of a coring cycle, an apparatus may be moved to a next position along a (vacuum) alignment frame.

In some embodiments, a spacer frame, such as a vacuum spacer frame (e.g. frame 10-4401 as shown in FIG. 29), may include one or more inner alignment elements. In some embodiments, a spacer frame may include a sub-frame, such as sub-frame 10-4405 as shown in FIG. 29, which can be used to align one or more sub-frame elements to a row of previously cored holes.

Optical technologies or devices may be used, such as to visually inspect a region of skin during coring and/or to align an apparatus (e.g. an apparatus 10-100, 10-200, or 10-400). In some embodiments, a spacer, such as a vacuum spacer including a frame, may be configured (e.g. sized) such that a region of skin being treated (e.g. cored) remains visible to a user during a procedure. In some embodiments, a spacer may include one or more structures that create a line of sight from a side of an apparatus and/or from a position proximal to an apparatus (e.g. a hand piece, such as hand piece 10-120, 10-220, or 10-420). In some embodiments, a spacer and/or spacer frame may be made from a transparent, semi-transparent and/or translucent material. In some embodiments, a spacer may include a mirror assembly (e.g. may include a mirror connected to a ball joint to adjust positioning and line of sight).

Optical devices and technologies may be used to align an apparatus (e.g. apparatus 10-100, 10-200, or 10-400), including, for example, light projection devices. In some embodiments, light projection devices may be used to project cross-hairs or other markings on a skin region, aiding visual alignment of an apparatus. Light projection technologies that may be used include light emitting diodes (LEDs), lasers, and/or other light emitters that may be used for unaided visual inspection or may be used with digital light processing techniques. In some embodiments, an apparatus as described herein may be aligned using direct visual inspection and/or using a vision system, for example using a camera and a display. In some embodiments, image processing systems and methods (e.g. implemented on a digital processing unit using data captured by an imaging system on an apparatus as described herein) may be used to guide a clinician or other user, such as by analyzing an already cored region, and these systems and methods may provide a user with guidance as to placement of an apparatus to core a next region. An image processing system may also be used to evaluate a coring procedure (e.g. in real time), such as to determine unsuccessful coring.

### Ingress Shield on Hand Piece

An apparatus as described herein (e.g. apparatus 10-100, 10-200, or 10-400) may include one or more single use components and/or one or more re-usable components. In some embodiments a needle hub may be a single-use component that is discarded, for example discarded after completion of a treatment procedure. In some embodiments, one or more components of an apparatus, such as components encased by a hand piece shell (e.g. hand piece shell 10-121, 10-221, or 10-421), may be re-usable. In some embodiments, a hand piece shell may be configured to be cleaned and/or sterilized. In some embodiments, a hand piece shell may be cleaned by wiping, such as during a cleaning procedure using ethanol or bleach. In some embodiments, a hand piece shell may be covered with a disposable drape during operation. The drape can be configured to allow actuator 120 (e.g. an x, y, z actuator) to move freely without compromising the fit of treatment module 150 to treatment device 100 (e.g. without adversely affecting the x, y, z movements).

As mentioned above, in some embodiments, a system as described herein may include technologies to prevent fluids or other substances from entering an apparatus, for example from entering a distal opening in a hand piece (e.g. hand piece 10-120, 10-220, or 10-420).

### Consumable Detection Hand Piece

In some embodiments, a needle hub (e.g. needle hub 10-110, 10-210, or 10-410, or needle hub 10-2710 or 10-3610) may be implemented as a consumable item, such as a needle hub that may be discarded after a certain amount of usage (e.g. after a particular number of procedures). In some embodiments, a needle hub may be replaced after a certain number of insertion/extraction cycles, such as after about 50 cycles, 100 cycles, 150 cycles, 200 cycles, 250 cycles, 300 cycles, 350 cycles, 400 cycles, 450 cycles, 500 cycles, 600 cycles, 700 cycles, 800 cycles, 900 cycles, or about 1000 cycles. In some embodiments, a needle hub may be replaced after a certain amount of time, such as an amount of time a needle hub is disposed in and/or on an apparatus (e.g. apparatus 10-100, 10-200, or 10-400), for example when mounted on a needle hub mount. In some embodiments, a needle hub may be a single use item, such as a needle hub that may not be used again once it has been removed from an apparatus (e.g. disconnected from a needle hub mount). This limitation of use may improve safety, such as by preventing re-use of a needle hub on a different subject, reducing likelihood of infection.

In some embodiments, a needle hub may include a unique identifier. In some embodiments, an identifier may be mounted on and/or integrated into a needle hub (e.g. a tag, a chip such as an RFID chip, for example when integrated in and/or on a secondary insert 10-2703 or 10-3603), and the identifier may be used to identify a specific needle hub (e.g. to monitor usage of the needle hub). In some embodiments, a tag may be mounted on a needle hub such that the tag may (directly) contact (e.g. touch) or may otherwise connect to an element (e.g. a read/write element), that is fixed to and/or is integrated into an apparatus (e.g. a needle hub mount, a hand piece and/or a z-actuator) when the needle hub is mounted (e.g. on a needle hub mount). In some embodiments, a read/write element may be fixed to and/or movably connected to a hand piece, such as when mounted on a hand piece shell (e.g. hand piece shell 10-121, 10-221, or 10-421). In some embodiments, a read/write element may be in electronic communication with a digital processing unit that may be operable to receive data from a needle hub tag (e.g. a chip), to process the received data, and/or to write the data to the needle hub tag. In some embodiments, a read/write element and needle hub tag may be implemented as a near field communication (NFC) system. In some embodiments, a needle hub tag may include data (e.g. electronic data) stored thereon, such as data encoding a unique identifier and/or a certain maximum number of cycles of use. In some embodiments, when a needle hub is mounted on, for example a needle hub mount, a digital processing unit may receive a signal that a needle hub is indeed mounted and may initiate data exchange with the tag via a read/write element. During coring, a digital processing unit may receive data from a z-actuator, such as via a sensor mounted thereon or from electric signals (e.g. data transfer) to and/or from a voice coil, and the processing unit may execute a program to count a number of insertion/extraction cycles that are performed (e.g. on one or more subjects). Once a certain number of cycles is reached, for example a threshold quantity pre-programmed into a digital processing unit and/or a threshold quantity stored on a needle hub tag, a digital processing unit may cause the z-actuator to cease moving, such as by blocking z-actuation until the needle hub is removed and a new needle hub is mounted. In some embodiments, a digital processing unit may cause a read/write element to write data (e.g. electronic data) to a tag on a needle hub, such as data indicating that the needle hub has been mounted and/or used. In some embodiments, a digital processing unit may cause a read/write element to write data to the tag, such as data indicating that the needle hub has been mounted and/or used, immediately after a needle hub is mounted on an apparatus. In some embodiments, a digital processing unit of an apparatus may be programmed to prevent z-actuation if such data indicating that the needle hub had been previously mounted is received, such as via a read write element. This programming may prevent the needle hub from being re-used once the needle hub has been dismounted.

In some embodiments, mounting of a needle hub (e.g. mounting a needle hub on a needle hub mount) may be verified, such as by using a Hall switch device including a Hall effect sensor. A Hall effect sensor is a transducer that may vary its output voltage in response to a magnetic field. In some embodiments, a needle hub may include a magnetic element (e.g. positioned at a proximal end) that is operable to activate a Hall effect sensor on, for example, a needle hub mount or hand piece when the needle hub is properly mounted on a hand piece and/or z-actuator. Upon mounting, a Hall effect switch device may receive a signal from the Hall effect sensor and transmit a signal to a digital processing unit (e.g. causing a z-actuator lock to be released).

In some embodiments, a reed switch device may be used instead of or in addition to a Hall switch device. A reed switch is an electrical switch activated by the presence of a magnetic field. In some embodiments, a needle hub may include a magnetic element (e.g. positioned at a proximal end) that is operable to activate a reed switch that is positioned on, for example, a needle hub mount (e.g. when the needle hub is properly mounted on, for example, a needle hub mount). A reed switch device may receive a signal from the reed switch and transmit a signal to a digital processing unit, e.g. causing a z-actuator lock to be released.

As described above, in some embodiments, a digital control unit may be programmed and/or used to detect potential damage to a needle hub and to block use of an apparatus (e.g. prevent actuation of a z-actuator) until the needle hub is properly replaced. In some embodiments, replacement of a damaged needle hub may be indicated by the removal of a tag (e.g. a chip) associated with a damaged needle hub and connection of a needle hub with a different tag (e.g. chip).

### Needles

An example apparatus of the present inventive concepts includes at least one hollow needle. In some embodiments, an example apparatus as described herein may include at least one hollow needle having at least a first prong. In some embodiments, an angle between a lateral side of a prong and a longitudinal axis of a hollow needle (e.g. a bevel angle a) may be at least about 20 degrees (e.g. the bevel angle a may be greater than about 20 degrees, such as greater than 20 degrees, 22 degrees, 24 degrees, 26 degrees, 28 degrees, 30 degrees, 32 degrees, 34 degrees, 36 degrees, 38 degrees, and 40 degrees, or at an angle of about 20 to about 40 degrees, between 20 to 40 degrees, 20 to 38 degrees, 20 to 36 degrees, 20 to 34 degrees, 20 to 32 degrees, 20 to 30 degrees, 20 to 28 degrees, 20 to 26 degrees, 20 to 24 degrees, 20 to 22 degrees, 22 to 40 degrees, 24 to 40 degrees, 26 to 40 degrees, 28 to 40 degrees, 30 to 40 degrees, 32 to 40 degrees, 34 to 40 degrees, 36 to 40 degrees, or 38 to 40 degrees). In particular, an angle between a lateral side of the prong and a longitudinal axis of the hollow needle (e.g. a bevel angle a) may be about 30 degrees.

In some embodiments, a tip of a prong of a hollow needle may be an edge. In some embodiments, a tip of a prong of a hollow needle is a flat tip having at least two dimensions. In some embodiments, a prong of a hollow needle includes a tip micro-feature. Hollow needles may be constructed to prevent frequent needle damage during use, such as needle tip curling and wear (e.g. becoming dull), needle heel degradation, and/or needle bending. Hollow needles may be designed to maintain mechanical integrity and durability over a large number of actuation cycles (e.g. actuation cycles greater than 500, 1,000, 2,000, 3,000, 4,000, 5,000, 6,000, 7,000, 8,000, 9,000, 10,000, 11,000, 12,000, 13,000, 14,000, 15,000, or 20,000). Needles may also effectively remove tissue portions from the skin with high coring rate. In some embodiments, to produce a cosmetic effect in skin tissue, a hollow needle of an apparatus may be inserted into the skin tissue, preferably to a pre-determined depth using a pre-determined force, such that a hollow needle removes a portion of the skin tissue by capturing the portion of the skin tissue in the lumen of the hollow needle.

### Prongs

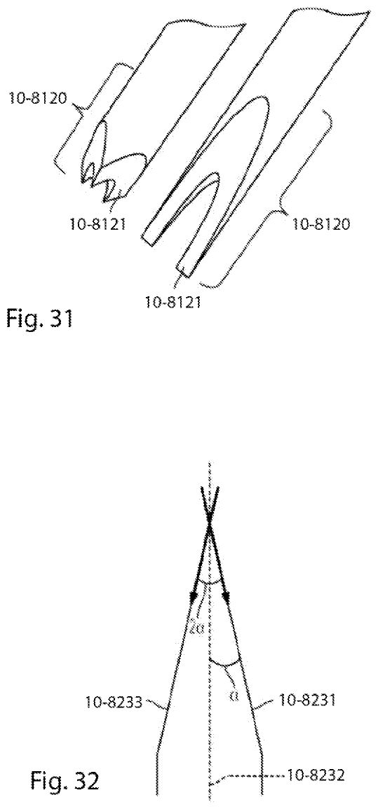

As shown in FIG. 31, distal end 10-8120 of a hollow needle of an apparatus (e.g. the end of the needle that penetrates the skin tissue) may be shaped to form one or more prongs 10-8121. In some embodiments, a hollow needle of an apparatus may have one prong at a distal end, two prongs, or more than two prongs (e.g. three, four, five, or six prongs). A hollow needle having one prong may be formed by grinding one side of a distal end of the hollow needle at an angle relative to a longitudinal axis of the hollow needle. A hollow needle having two prongs may be formed by grinding opposite sides of a distal end of the hollow needle at an angle relative to a longitudinal axis of the hollow needle.

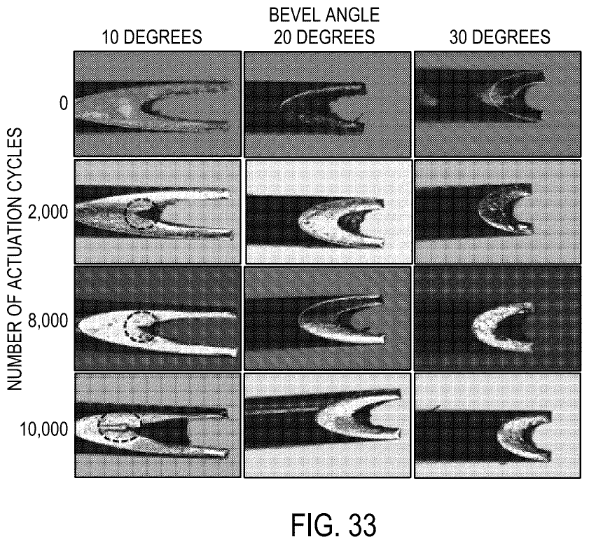

The geometry of a prong at a distal end of a hollow needle may be characterized by a bevel angle. A bevel angle, for example angle α as shown in FIG. 32, refers to the angle between lateral side 10-8231 of the prong and longitudinal axis 10-8232 of the hollow needle. An angle of “2α” refers to the angle between two lateral sides of the prong of a hollow needle, for example the angle between lateral side 10-8231 and lateral side 10-8233 of the hollow needle. In some embodiments, a bevel angle α between a lateral side of a prong and a longitudinal axis of the hollow needle may be at least about 20 degrees (e.g. between about 20 and about 40 degrees such as an angle of 20, 22, 24, 26, 28, 30, 32, 34, 36, 38, or 40 degrees). An angle between a lateral side of a prong and a longitudinal axis of a hollow needle may be about 30 degrees. For hollow needles having two or more prongs (e.g. as shown in FIG. 33), each prong may have the same bevel angle or different bevel angles. In some embodiments, for a hollow needle having two prongs, for example a first prong and a second prong, an angle between a lateral side of the first prong and a longitudinal axis of the hollow needle may be between about 20 and about 30 degrees (e.g. 20, 22, 24, 26, 28, or 30 degrees) and an angle between a lateral side of the second prong and a longitudinal axis of the hollow needle may be between about 30 and about 40 degrees (e.g. 30, 32, 34, 36, 38, or 40 degrees). For example, a first prong may have a bevel angle α of 20 degrees and a second prong may have a bevel angle α of 30 degrees.

A bevel angle α of at least about 20 degrees or more may improve the mechanical integrity of the needle over several actuation cycles of insertion and withdrawal into skin tissue. Table 1 below shows that a two-prong hollow needle having a 2α bevel angle of 40 degrees (the bevel angle α of each prong is 20 degrees) may reduce the occurrence of needle tip curling relative to a two-prong hollow needle having a 2α bevel angle of 20 degrees (the bevel angle α of each prong is 10 degrees). In an example implementation, a total of five two-prong hollow needles each having a bevel angle α of 10° and five two-prong hollow needles each having a bevel angle α of 20° were tested.

TABLE 1

| | |
|---|---|
| Number of | Number of Needles showing Tip Curling |

| Actuation Cycles | 10° Bevel Angle α | 20° Bevel Angle α |
|---|---|---|
| 5,000 | 1 | 0 |
| 10,000 | 2 | 0 |
| 15,000 | 2 | 0 |
| 20,000 | 3 | 1 |

Additionally, FIG. 33 shows that increasing a needle bevel angle α of a prong may also reduce occurrence of needle heel degradation over a large number of actuation cycles. As shown in FIG. 33, a hollow needle having a bevel angle α of 10 degrees displayed signs of needle heel degradation (indicated by dashed circles) before 2,000 actuation cycles, while a hollow needle having a bevel angle α of 20 degrees and a hollow needle having a bevel angle α of 30 degrees showed no apparent sign of needle heel degradation over 10,000 actuation cycles.

A tip of a prong of a hollow needle may be of varying geometries. For example, a tip of a prong may have a sharp point or an edge (e.g. a one-dimensional edge). In some embodiments, for a prong having an edge at the tip, each of the bevel angles of the prong may be at least about 20 degrees (e.g. from about 20 to about 40 degrees such as about 30 degrees). In some embodiments, for a hollow needle having two or more prongs (e.g. two prongs), the prongs may have different bevel angles (e.g. a bevel angle α of about 20 degrees at the first prong and a bevel angle α of about 30 degrees at the second prong). A tip of a prong may be a flat tip (e.g. a flat tip having two dimensions). For example, a flat tip may have a length and a width. A surface (length/width) of the flat tip of the prong may be at an angle relative to the longitudinal axis of the hollow needle. For example, the surface of the flat tip may be perpendicular to the longitudinal axis of the hollow needle (e.g. at a 90 degree angle relative to the longitudinal axis of the hollow needle) or the surface of the flat tip may be at a non-90 degree angle relative to the longitudinal axis of the hollow needle (e.g. between about 3 to about 89 degrees, such as 3 to 89 degrees, such as 3, 6, 9, 12, 15, 18, 21, 24, 27, 30, 33, 36, 39, 42, 45, 48, 51, 54, 57, 60, 63, 66, 69, 72, 75, 78, 81, 84, 87, or 89 degrees). A surface of a flat tip may be level or may have a different geometry, for an arc, groove, and/or non-level geometry. For a prong having a two-dimensional flat tip, each of the bevel angles of the prong may be between about 2 degrees to about 40 degrees (e.g. 2, 4, 6, 8, 10, 12, 14, 16, 18, 20, 22, 24, 26, 28, 30, 32, 34, 36, 38, or 40 degrees). A needle may have one or two prongs each with a two-dimensional flat tip in which one or both of the prongs have a bevel angle α of at least about 20 degrees (e.g. from about 20 to about 40 degrees (e.g. about 30 degrees)). Needles having a one-dimensional edge or a two-dimensional flat tip may exhibit a reduced likelihood of needle tip curling.

### Gauges: Inner Diameters and Lengths

A hollow needle of an apparatus described herein may be of any gauge, including gauges of from 18 to 30 (e.g. 18, 19, 20, 21, 22, 23, 24, 25, 26, 27, 28, 29, or 30 gauge). The gauges of a hollow needle may be from 22 to 25 (e.g. 22, 23, 24, or 25 gauge). A hollow needle of the apparatus may have an inner diameter of from about 0.14 mm to about 0.84 mm (e.g. 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, or 0.84 mm). An inner diameter of a hollow needle may refer to the diameter of the inner lumen of the hollow needle. An inner diameter of a hollow needle may be from about 0.24 mm to about 0.40 mm (e.g. 0.24, 0.25, 0.26, 0.27, 0.28, 0.29, 0.3, 0.31, 0.32, 0.33, 0.34, 0.35, 0.36, 0.37, 0.38, 0.39, or 0.4 mm). An inner diameter of a hollow needle may be from about 0.5 mm to about 2.5 mm (e.g. 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 mm). Accordingly, in some embodiments, a diameter of a portion of skin tissue removed by a hollow needle of an apparatus (e.g. a cored tissue portion) may generally correspond to an inner diameter of a hollow needle.

In some embodiments, an outer and/or inner diameter of a hollow needle may vary across its length, such that the diameter of one region of a hollow needle may be different from the outer and/or inner diameter of another region of the same needle. A change in a diameter across a hollow needle may or may not be continuous. In some embodiments, a hollow needle may or may not be entirely cylindrical. For example, one or more hollow needles may be rectangular, serrated, scalloped, and/or irregular in one or more dimensions and along some or all of their lengths. In some embodiments, the inner lumen diameter may vary along the length of a hollow needle. In some embodiments, a needle may be a swaged hollow needle having a bevel angle α of at least 20 degrees (e.g. between about 20 and about 40 degrees (e.g. 20, 22, 24, 26, 28, 30, 32, 34, 36, 38, or 40 degrees)) and a variable inner lumen diameter over its length. A swaged hollow needle may have a smaller diameter near the distal end of the hollow needle (e.g. near the end of the needle that penetrates the skin tissue). In some embodiments, an inner diameter may be wider at the proximal end of a hollow needle (e.g. away from the tip that penetrates the skin). This may facilitate the removal of a cored tissue portion from the hollow needle, may limit the need for clearing of the hollow needle, and/or may reduce the occurrence of needle clogging.

A hollow needle of an apparatus may be of varying lengths and may have varying active lengths (e.g. the length of a hollow needle configured to penetrate the skin tissue). Active lengths may vary from about 0.5 mm to about 10 mm (e.g. 0.5, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, or 10 mm) and may be adjustable/selectable with manual or automatic controls (e.g. as described herein, such as using a scroll wheel and/or an actuation mechanism such as an electromagnetic actuator). Active lengths of a hollow needle may be adjusted and selected depending on a skin area needing treatment. In some embodiments, a hollow needle with an active length from about 0.5 mm to about 2 mm (e.g. 0.5, 0.6, 0.8, 1, 1.2, 1.4, 1.6, 1.8, or 2 mm) may be used to treat thin skin, for example skin of an eyelid. The thickness of the epidermal and dermal layers of the skin of an eyelid may be from about 0.5 mm to about 1 mm (e.g. 0.5, 0.6, 0.8, or 1 mm). Hollow needles with active lengths from about 5 mm to about 10 mm (e.g. 5, 6, 7, 8, 9, or 10 mm) may be used to treat thick skin, for example skin of the back or scar tissue, which may be thicker than healthy skin tissue. The thickness of an epidermal layer of skin may be from about 0.05 mm to about 2 mm (e.g. 0.05 to 2, 0.05 to 1.95, 0.05 to 1.9, 0.05 to 1.85, 0.05 to 1.8, 0.05 to 1.75, 0.05 to 1.7, 0.05 to 1.65, 0.05 to 1.6, 0.05 to 1.55, 0.05 to 1.5, 0.05 to 1.45, 0.05 to 1.4, 0.05 to 1.35, 0.05 to 1.3, 0.05 to 1.25, 0.05 to 1.2, 0.05 to 1.15, 0.05 to 1.1, 0.05 to 1.05, 0.05 to 1, 0.05 to 0.95, 0.05 to 0.9, 0.05 to 0.85, 0.05 to 0.8, 0.05 to 0.75, 0.05 to 0.7, 0.05 to 0.65, 0.05 to 0.6, 0.05 to 0.55, 0.05 to 0.5, 0.05 to 0.45, 0.05 to 0.4, 0.05 to 0.35, 0.05 to 0.3, 0.05 to 0.25, 0.05 to 0.2, 0.05 to 0.15, 0.05 to 0.1, 0.1 to 2, 0.15 to 2, 0.2 to 2, 0.25 to 2, 0.3 to 2, 0.35 to 2, 0.4 to 2, 0.45 to 2, 0.5 to 2, 0.55 to 2, 0.6 to 2, 0.65 to 2, 0.7 to 2, 0.75 to 2, 0.8 to 2, 0.85 to 2, 0.9 to 2, 0.95 to 2, 1 to 2, 1.05 to 2, 1.15 to 2, 1.2 to 2, 1.25 to 2, 1.3 to 2, 1.35 to 2, 1.4 to 2, 1.45 to 2, 1.5 to 2, 1.55 to 2, 1.6 to 2, 1.65 to 2, 1.7 to 2, 1.75 to 2, 1.8 to 2, 1.85 to 2, 1.9 to 2, or 1.95 to 2 mm). The thickness of a dermal layer of skin may be from 2 to 8 mm (e.g. 2 to 8, 2 to 7.5, 2 to 7, 2 to 6.5, 2 to 6, 2 to 5.5, 2 to 5, 2 to 4.5, 2 to 4, 2 to 3.5, 2 to 3, 2 to 2.5, 2.5 to 8, 3 to 8, 3.5 to 8, 4 to 8, 4.5 to 8, 5 to 8, 5.5 to 8, 6 to 8, 6.5 to 8, 7 to 8, or 7.5 to 8 mm). Active lengths of a hollow needle may be adjusted and selected to penetrate the epidermal and/or the dermal layer of skin.

In some embodiments, active lengths of a hollow needle may also be adjusted using one or more spacers, which are described in detail further herein. Hollow needle parameters may be selected based on the area of skin and the condition of the skin to be treated. For example, treatment of thin, lax skin on the cheeks may benefit from a hollow needle having an active length of about 2 mm and medium gauge (e.g. 25 gauge), while treatment of thick skin on the back or treatment of scar tissue may benefit from a hollow needle having an active length closer to 5 mm and a thicker gauge (e.g. 22 gauge). A hollow needle of an apparatus may be configured to extend to varying depths of the skin tissue. In some embodiments, depth of penetration of a hollow needle may be determined by the active length (e.g. from about 2 mm to about 5 mm) of a hollow needle. In some embodiments, a hollow needle may be configured to extend (i) into the dermal layer, (ii) through the entire dermal layer to the junction of the dermal layer and the subcutaneous fat layer, and/or (iii) into the subcutaneous fat layer.

### Surface of Needle Lumen

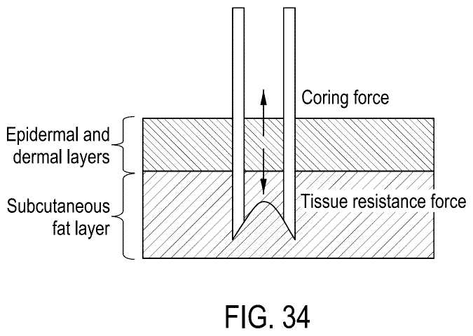

A lumen surface of a hollow needle may affect coring force, coring rate, and/or insertion force of the hollow needle. Without intending to be bound by theory, the friction between a lumen surface and a cored tissue portion may determine the coring force, coring rate, and/or insertion force. Hollow needles described herein may be designed to maximize coring rate and minimize hollow needle insertions that do not result in cored tissue removal. A tissue portion detaches from skin when a coring force (e.g. the force applied by the hollow needle of the apparatus to the cored tissue portion as the needle is being withdrawn from the skin) exceeds a tissue resistance force, which may be determined by the connection of the tissue portion to its surrounding tissue. For example, when a hollow needle is fully inserted through the dermal layer of the skin, a tissue resistance force may be determined by the connection between the tissue portion in the lumen of the needle and the subcutaneous fat layer. Accordingly, when coring force exceeds tissue resistance force, the cored tissue portion may be captured in the lumen of the hollow needle and removed from the skin (reference FIG. 34). A rough lumen surface may increase friction between a cored tissue portion and a lumen surface, which may result in increased insertion force, increased coring force, and/or increased coring rate. Lubrication of a lumen surface may reduce friction between a cored tissue portion and a lumen surface, which may result in decreased insertion force, decreased coring force, and decreased coring rate. An overly rough and uneven lumen surface may lead to higher occurrence of needle degradation (e.g. needle heel and/or tip degradations), may cause difficulty in removing cored tissue portions from a lumen, and/or may cause needle clogging, compared to a needle having a smooth and/or even lumen surface. The degree of roughness of a lumen surface may be optimized to increase coring force and/or coring rate without compromising the durability of the needle, the insertion force, the ability to remove tissue from the needle lumen, and the resistance of a needle to degradation (e.g. needle heel and tip degradation).

In some embodiments, hollow needles and methods may have a coring rate of at least about 5% (e.g. from about 5% to about 100%, such as 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 95%, 15% to 95%, 20% to 95%, 25% to 95%, 30% to 95%, 35% to 95%, 40% to 95%, 45% to 95%, 50% to 95%, 55% to 95%, 60% to 95%, 65% to 95%, 70% to 95%, 75% to 95%, 80% to 95%, 85% to 95%, or 90% to 95%).

In some embodiments, hollow needles and methods may exert a coring force of about 3 N to about 10 N (e.g. 3, 3.5, 4, 4.5, 5, 5.5, 6, 6.5, 7, 7.5, 8, 8.5, 9, 9.5, or 10 N). In some embodiments, a two-prong hollow needle having a bevel angle α of 20 degrees may exert a coring force of about 3 N to about 10 N (e.g. 3, 3.5, 4, 4.5, 5, 5.5, 6, 6.5, 7, 7.5, 8, 8.5, 9, 9.5, or 10 N).

A coating material and/or a lubricant may affect the degree of roughness of the lumen surface, and thus friction between the lumen surface and a cored tissue portion. A lumen surface of a hollow needle may be polished by running a lubricant or polishing media through the hollow needle to reduce the roughness of the lumen surface. Examples of lubricants include, but are not limited to, salt-based lubricants (e.g. buffered saline solutions, such as PBS), sugar-based lubricants (e.g. sucrose and glucose solutions), and/or surfactant-based lubricants (e.g. solutions containing Tween20). The degree of roughness of the lumen surface of the hollow needle may also be affected by the manufacturing process used to make the hollow needle. Table 2 below shows lumen surface roughness measured in Ra (arithmetic average of roughness profile) and Rz (mean roughness depth) of hollow needles made using single plug, double plug, and/or sunk manufacturing processes. The lumen surface of hollow needles made using double plug process may be smoother (lower Ra and Rz values) than the lumen surface of hollow needles made using single plug process.

TABLE 2

| Manufacturing Process | Ra | Rz |
|---|---|---|
| Single plug | 53 | 299 |
| Double plug | 37 | 206 |
| Sunk | 56 | 330 |

### Array Patterns

One or more hollow needles of a system and/or apparatus of the present inventive concepts may be arranged (e.g. on a needle hub) to form an array pattern in skin upon removal of portions of skin tissue. In some embodiments, an array pattern may include holes in one or more rows or in a random or semi random spatial distribution. Size and geometry of an array pattern may be generated based on an area of skin and condition being treated. In some embodiments, a small array pattern may be generated for treatment of the peri-oral area, while a large array pattern may be suitable for treatment of the abdomen. In some embodiments, an array pattern may be generated using different numbers and/or arrangements of a plurality of hollow needles. In some embodiments, an array pattern may be generated using one hollow needle, which may undergo multiple actuation cycles and be translated across a surface of a skin region, such as by an x-actuator and/or y-actuator to generate an array pattern. In some embodiments, an array pattern may be generated using a plurality of hollow needles (e.g. an array of hollow needles), which may undergo one or more actuation cycles to generate an array pattern. A number of actuation cycles needed to generate an array pattern of holes in skin tissue may be determined by the size of the array pattern, the gauge and/or inner and/or outer diameter of a hollow needle, the number of hollow needles, the size distribution of a plurality of needles of different sizes, and/or an amount of skin tissue to be removed, for example an areal fraction of skin tissue removed. An “areal fraction” of tissue removed refers to the fraction of skin tissue surface covered by holes generated by one or more hollow needle(s) of an apparatus. In other words, an areal fraction of tissue removed refers to the ratio of the area covered by the total amount of cored tissue portions to the total skin treatment area. In some embodiments, one or more hollow needles may be used or configured to remove an areal fraction of about 0.01 to about 0.65 (e.g. 0.01, 0.02, 0.03, 0.04, 0.05, 0.10, 0.15, 0.20, 0.25, 0.30, 0.35, 0.40, 0.45, 0.50, 0.55, 0.60, or 0.65) of tissue within a treatment area. In some embodiments, one or more hollow needles may be used or configured to remove an areal fraction of less than about 0.1, such as about 0.01 to about 0.05 (e.g. 0.01, 0.015, 0.02, 0.025, 0.03, 0.035, 0.04, 0.045, or 0.05) of tissue within a treatment area. In some embodiments, one or more hollow needles may be used or configured to remove an areal fraction of about 0.02 to about 0.03 (e.g. 0.02, 0.021, 0.022, 0.023, 0.024, 0.025, 0.026, 0.027, 0.028, 0.029, or 0.03, e.g. 0.025) of tissue within a treatment area. In some embodiments, an areal fraction of about 0.01 to about 0.65 (e.g. 0.01, 0.02, 0.03, 0.04, 0.05, 0.10, 0.15, 0.20, 0.25, 0.30, 0.35, 0.40, 0.45, 0.50, 0.55, 0.60, or 0.65) of tissue may be removed within a treatment area, for example for wrinkle reduction. In some embodiments, an areal fraction of about 0.02 to about 0.03 (e.g. 0.02, 0.021, 0.022, 0.023, 0.024, 0.025, 0.026, 0.027, 0.028, 0.029, or 0.03, e.g. 0.025) of tissue may be removed within a treatment area, for example for wrinkle reduction. Table 3 below shows an example number of actuation cycles required for the treatment of different body areas using a 24 gauge hollow needle.

TABLE 3

| Treatment | Total Treatment | Areal Fraction of | Number of |
|---|---|---|---|
| Site | Area (cm²) | Tissue Removed | Actuation Cycles |
| Cheek | 120 | 0.1 | 15,782 |
| Upper lip | 10 | 0.1 | 1,315 |
| Knee | 120 | 0.1 | 15,782 |
| Hand | 100 | 0.1 | 13,151 |

An apparatus of the present inventive concepts may be configured for detachable attachment to one or more hollow needles having the same or different configurations. In some embodiments, an apparatus may have as few as 1 or as many as hundreds of hollow needles. In some embodiments, 1-100 hollow needles may be present (e.g. 1-10, 1-20, 1-30, 1-40, 1-50, 1-60, 1-70, 1-80, 1-90, 1-100, 3-10, 3-20, 3-30, 3-40, 3-50, 3-60, 3-70, 3-80, 3-90, 3-100, 5-10, 5-20, 5-30, 5-40, 5-50, 5-60, 5-70, 5-80, 5-90, 5-100, 10-20, 10-40, 10-60, 10-80, 10-100, 20-40, 20-60, 20-80, 20-100, 40-60, 40-80, 40-100, 60-80, 60-100, or 80-100 hollow needles). The use of an array of a plurality of hollow needles to generate an array pattern may facilitate skin treatment over larger areas and/or in less time.

In some embodiments, a minimum distance between two hollow needles in an array of hollow needles may be between about 0.1 mm to about 50 mm (e.g. from 0.1 mm to 0.2 mm, 0.1 mm to 0.5 mm, 0.1 mm to 1 mm, 0.1 mm to 2 mm, 0.1 mm to 5 mm, 0.1 mm to 10 mm, 0.1 mm to 15 mm, 0.1 mm to 20 mm, 0.1 mm to 30 mm, 0.1 mm to 40 mm, 0.1 mm to 50 mm, 0.2 mm to 0.5 mm, 0.2 mm to 1 mm, 0.2 mm to 2 mm, 0.2 mm to 5 mm, 0.2 mm to 10 mm, 0.2 mm to 15 mm, 0.2 mm to 20 mm, 0.2 mm to 30 mm, 0.2 mm to 40 mm, 0.2 mm to 50 mm, 0.5 mm to 1 mm, 0.5 mm to 2 mm, 0.5 mm to 5 mm, 0.5 mm to 10 mm, 0.5 mm to 15 mm, 0.5 mm to 20 mm, 0.5 mm to 30 mm, 0.5 mm to 40 mm, 0.5 mm to 50 mm, 1 mm to 2 mm, 1 mm to 5 mm, 1 mm to 10 mm, 1 mm to 15 mm, 1 mm to 20 mm, 1 mm to 30 mm, 1 mm to 40 mm, 1 mm to 50 mm, 2 mm to 5 mm, 2 mm to 10 mm, 2 mm to 15 mm, 2 mm to 20 mm, 2 mm to 30 mm, 2 mm to 40 mm, 2 mm to 50 mm, 5 mm to 10 mm, 5 mm to 15 mm, 5 mm to 20 mm, 5 mm to 30 mm, 5 mm to 40 mm, 5 mm to 50 mm, 10 mm to 15 mm, 10 mm to 20 mm, 10 mm to 30 mm, 10 mm to 40 mm, 10 mm to 50 mm, 15 mm to 20 mm, 15 mm to 30 mm, 15 mm to 40 mm, 15 mm to 50 mm, 20 mm to 30 mm, 20 mm to 40 mm, 20 mm to 50 mm, 30 mm to 40 mm, 30 mm to 50 mm, or 40 mm to 50 mm). In some embodiments, a distance between two hollow needles in an array of hollow needles is less than about 15 mm. In some embodiments, a minimum distance may correspond to the minimum size of an array pattern, while the maximum distance may correspond to the maximum size or dimension of an array pattern.

Coring procedures may be adapted and/or optimized, such as to adapt coring to specific tissue types (e.g. wrinkles, scars, and/or dog ears), and/or to trace certain features (e.g. scars and/or tumors). Coring depth, hole density, and/or patterns may be adapted and/or optimized. In some embodiments, array patterns of different sizes and geometries may be generated based on the area of treatment and the skin condition being treated. In some embodiments, array patterns may also be generated for compatibility with actuation mechanisms and/or control electronics of a given apparatus. In some embodiments, actuation mechanisms and/or control electronics of an apparatus may be selected for compatibility with a desired array pattern size and/or geometry. In some embodiments, a long, linear array pattern may be generated using a translating mechanism with driving wheels, while a large, rectangular array may be generated using an x- and/or y-actuator to drive the hollow needle(s) across skin. In some embodiments, a pattern may be pre-programmed or adapted during a procedure, for example during a coring process, such as to adapt and/or optimize treatment in real time. In some embodiments, adaptation and/or optimization of a coring procedure may be based on tissue characteristics. In some embodiments, adaptation and/or optimization may be carried out based on voice coil data (e.g. kinematics and/or electronics), and/or it may be carried out based on other data, such as acoustic, optical, and/or radiofrequency data obtained before, during, and/or after a coring procedure.

In an example apparatus, one or more hollow needles may be configured to provide from about 10 to about 10000 cored tissue portions or more per cm² area (e.g. 10 to 50, 10 to 100, 10 to 200, 10 to 300, 10 to 400, 10 to 500, 10 to 600, 10 to 700, 10 to 800, 10 to 900, 10 to 1000, 10 to 2000, 10 to 4000, 10 to 6000, 10 to 8000, 10 to 10000, 50 to 100, 50 to 200, 50 to 300, 50 to 400, 50 to 500, 50 to 600, 50 to 700, 50 to 800, 50 to 900, 50 to 1000, 50 to 2000, 50 to 4000, 510 to 6000, 50 to 8000, 50 to 10000, 100 to 200, 100 to 300, 100 to 400, 100 to 500, 100 to 600, 100 to 700, 100 to 800, 100 to 900, 100 to 1000, 100 to 2000, 100 to 4000, 100 to 6000, 100 to 8000, 100 to 10000, 200 to 300, 200 to 400, 200 to 500, 200 to 600, 200 to 700, 200 to 800, 200 to 900, 200 to 1000, 200 to 2000, 200 to 4000, 200 to 6000, 200 to 8000, 200 to 10000, 300 to 400, 300 to 500, 300 to 600, 300 to 700, 300 to 800, 300 to 900, 300 to 1000, 300 to 2000, 300 to 4000, 300 to 6000, 300 to 8000, 300 to 10000, 400 to 500, 400 to 600, 400 to 700, 400 to 800, 400 to 900, 400 to 1000, 400 to 2000, 400 to 4000, 400 to 6000, 400 to 8000, 400 to 10000, 500 to 600, 500 to 700, 500 to 800, 500 to 900, 500 to 1000, 500 to 2000, 500 to 4000, 500 to 6000, 500 to 8000, 500 to 10000, 600 to 700, 600 to 800, 600 to 900, 600 to 1000, 600 to 2000, 600 to 4000, 600 to 6000, 600 to 8000, 600 to 10000, 700 to 800, 700 to 900, 700 to 1000, 700 to 2000, 700 to 4000, 700 to 6000, 700 to 8000, 700 to 10000, 800 to 900, 800 to 1000, 800 to 2000, 800 to 4000, 800 to 6000, 800 to 8000, 800 to 10000, 900 to 1000, 900 to 2000, 900 to 4000, 900 to 6000, 900 to 8000, 900 to 10000, 1000 to 2000, 1000 to 4000, 1000 to 6000, 1000 to 8000, 1000 to 10000, 2000 to 4000, 2000 to 6000, 2000 to 8000, 2000 to 10000, 4000 to 6000, 4000 to 8000, 4000 to 10000, 6000 to 8000, 6000 to 10000, or 8000 to 10000 tissue portions per cm² area) of the skin region to which the apparatus is applied (e.g. the treatment area).

### Base Unit and User Interface

An apparatus as described herein (e.g. apparatus 10-100, 10-200, or 10-400) may be in communication with a base unit and/or control unit, which may include, for example, a user interface, a power supply, control electronics (e.g. a digital processing unit), mechanisms to drive operation of the apparatus, and/or other components. A base unit may include a computer including, for example, a digital processing unit, which may be programmed to operate and/or control any or all aspects of a system or an apparatus (e.g. apparatus 10-100, 10-200, or 10-400) as described herein. A base unit may include one or more pumps, valves, traps, actuators, switches, and/or tubing, such as to generate low pressure or (partial) vacuum in a system and/or to move fluids through one or more components of a system and/or apparatus.

A user interface in a base unit may include buttons, keys, switches, toggles, spin-wheels, screens, touch screens, keyboards, cursors, dials, indicators, displays, and/or other components, and the user interface may be connected to one or more digital processing units. In some embodiments, a user interface may be configured and/or programmed to indicate proper couplings and/or attachments of one or more components of a system, for example to indicate proper couplings and/or attachments of a support base, a z-actuator (e.g. a voice coil), one or more hollow needles, a fluid conduit, an aspiration tube, a trap, a low pressure and/or (partial) vacuum generation system, a pressure generating source (e.g. a vacuum pump), and or a needle assembly. In some embodiments, a user interface may be configured and/or programmed to indicate, for example, charged and/or powered status of an apparatus, mode and/or position of hollow needle(s), application of high (e.g. positive) pressure or low pressure (e.g. partial vacuum), actuation of one or more apparatus components, and/or other indicia. In some embodiments, a user interface may be configured and/or programmed to provide information about the number and/or kind of hollow needle(s) of an apparatus, a treatment area, treatment coverage (e.g. areal fraction of skin surface area removed), arrangement of one or more hollow needles, potential depth of penetration by hollow needle(s), mechanism and/or mode of operation, use count of the hollow needle(s), and/or other information. In some embodiments, a user interface may include implements for adjustment of parameters and/or operation mode, application of high (e.g. positive) pressure or low pressure (e.g. partial vacuum), and/or activation of penetration into the skin by one or more hollow needle(s). In some embodiments, a user interface may also be configured or programmed to transmit and/or receive information from another unit (e.g. another component of the system of the present inventive concepts). For example, user actions at a user interface on an apparatus may be reflected by a user interface of the base unit, or vice versa.

A base unit may include buttons, keys, switches (e.g. hand switches or foot switches), toggles, spin-wheels, and/or other activation mechanisms (e.g. user input controls) which can be configured for: adjustment of parameters and/or operational modes; adjustment of pressure, such as application of high (e.g. positive) pressure or low pressure (e.g. partial vacuum); adjustment of depth and/or duration of penetration into skin by one or more hollow needle(s); and/or powering on and/or off of a base unit and/or pressure generating source. In some embodiments, these components may be integrated into a user interface of the base unit. In some embodiments, a base unit may include one or more foot switches that may allow a user to operate one or more functions of a system, for example, low pressure system and/or z-actuation without use of a user's hands, such as while maintaining grip on a hand piece. In some embodiments, one or more feedback devices and/or controls may be integrated into an apparatus (e.g. a hand piece) and may include lights, screens, vibrating implements, and/or audio signal generators.

In some embodiments, the base unit may include electronics (e.g. electronic components and/or assemblies) to control operation of the apparatus, pressure generating source, and/or other components operably coupled to the apparatus. For example, the base unit may include one or more microcontrollers, programmable logic, discrete elements, and/or other components. The base unit may have one or more power supplies, and/or it may include one or more connections to a power supply external to the base unit. Power supplies may include batteries, capacitors, alternators, generators, and/or other components. In some embodiments, a base unit may include one or more devices for conversion of main power (alternating current provided by an electrical outlet) to direct current for system operation. In some embodiments, a base unit may include a battery charging station for use with a battery-powered apparatus.

In some embodiments, a base unit may include a user interface that may indicate, for example, that a hollow needle is properly installed in a needle hub, that a needle hub is properly coupled to an actuation unit, that an apparatus is charged or otherwise powered (e.g. the amount of battery life remaining), that one or more hollow needles are in an extended or retracted position, that a pressure generating source is coupled to an apparatus, that a fill level of a trap for collecting cored tissue portions has been reached, and/or other information. In some embodiments, a user interface may include information about an apparatus, such as the number of hollow needle(s) of the apparatus, an arrangement (e.g. a current arrangement) of the hollow needle(s), a potential depth of tissue penetration by the hollow needle(s), a mechanism and/or mode of operation, and/or other information. In some embodiments, a user interface may include buttons, keys, switches, toggles, spin-wheels, LED displays, and/or touch screens that allow a user to observe and change various parameters or configurations during operation of the apparatus, to activate and/or deactivate a pressure generating source, and/or to initiate penetration into the skin by one or more hollow needle(s). In some embodiments, a user interface may also be configured to transmit and/or receive information from another unit (e.g. another component of the system of the present inventive concepts), such as a computer (e.g. a digital processing unit).

In some embodiments, a base unit is or comprises a cart for example a cart including a structure moveable (e.g. on wheels). In some embodiments, one or more pumps, traps, and/or user interfaces are mounted on a cart. In some embodiments, an apparatus is connected to a base unit, such as a cart, via a moveable articulated arm, for example to support an apparatus or hand piece and/or to facilitate movement and/or stabilization of an apparatus or hand piece.

### Materials

The technologies described herein (e.g. hollow needles, needle hubs, actuation units, apparatuses, kits, and methods described herein) may include (e.g. be comprised of/made from) any material. For example, a needle hub may include and/or be formed from any polymer or plastic. Such materials may include alginate, benzyl hyaluronate, carboxymethylcellulose, cellulose acetate, chitosan, collagen, dextran, epoxy, gelatin, hyaluronic acid, hydrocolloids, nylon (e.g. nylon 6 or PA6), pectin, poly (3-hydroxyl butyrate-co-poly (3-hydroxyl valerate), polyalkanes, polyalkene, polyalkynes, polyacrylate (PA), polyacrylonitrile (PAN), polybenzimidazole (PBI), polycarbonate (PC), polycaprolactone (PCL), polyester (PE), polyethylene glycol (PEG), polyethylene oxide (PEO), PEO/polycarbonate/polyurethane (PEO/PC/PU), poly(ethylene-co-vinyl acetate) (PEVA), PEVA/polylactic acid (PEVA/PLA), polyethylene, polypropylene, poly (ethylene terephthalate) (PET), PET/poly (ethylene naphthalate) (PET/PEN) polyglactin, polyglycolic acid (PGA), polyglycolic acid/polylactic acid (PGA/PLA), polyimide (PI), polylactic acid (PLA), poly-L-lactide (PLLA), PLLA/PC/polyvinylcarbazole (PLLA/PC/PVCB), poly (b-malic acid)-copolymers (PMLA), polymethacrylate (PMA), poly (methyl methacrylate) (PMMA), polystyrene (PS), polyurethane (PU), poly (vinyl alcohol) (PVA), polyvinylcarbazole (PVCB), polyvinyl chloride (PVC), polyvinylidenedifluoride (PVDF), polyvinylpyrrolidone (PVP), silicone, rayon, polytetrafluoroethylene (PTFE), polyether ether ketone (PEEK), or combinations thereof. Polymers and/or plastics that may be used in the apparatus or system as described herein may be composite materials in which additives to the polymers and/or plastics, such as ceramics or particles, alter the mechanical properties.

Elements of the technologies described herein (e.g. all or a portion of the apparatus, such as all or a portion of the needle assembly, the actuation unit, or other components) may also include and/or be formed from any useful metal or metal alloy. For example, in some embodiments, a hollow needle may be a metallic needle. Metals and alloys that may be used in the apparatus or system as described herein include stainless steel; titanium; a nickel-titanium (NiTi) alloy; a nickel-titanium-niobium (NiTiNb) alloy; a nickel-iron-gallium (NiFeGa) alloy; a nickel-manganese-gallium (NiMnGa) alloy; a copper-aluminum-nickel (CuAlNi) allow; a copper-zinc (CuZn) alloy; a copper-tin (CuSn) alloy; a copper-zinc-aluminum (CuZnAl) alloy; a copper-zinc-silicon (CuZnSi) alloy; a copper-zinc-tin (CuZnSn) alloy; a copper-manganese alloy; a gold-cadmium (AuCd) alloy; a silver-cadmium (AgCd) alloy; an iron-platinum (FePt) alloy; an iron-manganese-silicon (FeMnSi) alloy; a cobalt-nickel-aluminum (CoNiAl) alloy; a cobalt-nickel-gallium (CoNiGa) alloy; a titanium-palladium (TiPd) alloy or combinations thereof. Elements of the technologies described herein may include and/or be formed from glass. For example, an apparatus may include one or more glass hollow needles.

The systems, hollow needles, needle assemblies, actuation units, apparatuses, kits, and/or methods described herein may include one or more adhesives. An adhesive may be located on a surface, between elements, or otherwise adhered to an element, e.g. of an apparatus as described herein. Example adhesives include a biocompatible matrix (e.g. those including at least one of collagen, such as a collagen sponge, low melting agarose (LMA), polylactic acid (PLA), and/or hyaluronic acid, such as hyaluranon; a photosensitizer, such as Rose Bengal, riboflavin-5-phosphate (R-5-P), methylene blue (MB), N-hydroxypyridine-2-(IH)-thione (N-HTP), a porphyrin, or a chlorin, as well as precursors thereof; a photochemical agent (e.g. 1,8 naphthalimide); a synthetic glue (e.g. a cyanoacrylate adhesive, a polyethylene glycol adhesive, or a gelatin-resorcinol-formaldehyde adhesive); a biologic sealant (e.g. a mixture of riboflavin-5-phosphate and fibrinogen, a fibrin-based sealant, an albumin-based sealant, or a starch-based sealant); and/or a hook or loop and eye system (e.g. as used for Velcro®)). In some embodiments, an included adhesive is biodegradable.

In some embodiments, an adhesive may be a pressure-sensitive adhesive (PSA). The properties of pressure sensitive adhesives can be governed by three parameters: tack (initial adhesion), peel strength (adhesion), and shear strength (cohesion). Pressure-sensitive adhesives can be synthesized in several ways, including solvent-borne, water-borne, and/or hot-melt methods. Tack is the initial adhesion under slight pressure and short dwell time and depends on the adhesive's ability to wet the contact surface. Peel strength is the force required to remove the PSA from the contact surface. The peel adhesion can depend on many factors, including the tack, bonding history (e.g. force, dwell time), and adhesive composition. Shear strength is a measure of the adhesive's resistance to continuous stress. The shear strength is influenced by several parameters, including internal adhesion, cross-linking, and viscoelastic properties of the adhesive. Permanent adhesives are generally resistant to debonding and possess very high peel and shear strength. Pressure-sensitive adhesives may include natural rubber, synthetic rubber (e.g. styrene-butadiene and styrene-ethylene copolymers), polyvinyl ether, polyurethane, acrylic, silicones, and ethylene-vinyl acetate copolymers. A copolymer's adhesive properties can be altered by varying the composition (via monomer components), changing the glass transition temperature (Tg) or degree of cross-linking. In general, a copolymer with a lower Tg is less rigid and a copolymer with a higher Tg is more rigid. The tack of PSAs can be altered by the addition of components to alter the viscosity or mechanical properties. Pressure sensitive adhesives are further described in Czech et al, “Pressure-Sensitive Adhesives for Medical Applications,” in Wide Spectra of Quality Control, Dr. Isin Akyar (Ed., published by InTech), Chapter 17 (2011), which is hereby incorporated by reference in its entirety.

A system, apparatus, method, and/or kit of the present inventive concepts may contain or be used to deliver one or more useful therapeutic agents. For example, the hollow needles of an apparatus as described herein may be configured to administer one or more therapeutic agents to the skin. In some embodiments, hollow needles of an apparatus as described herein may be used to create direct channels or holes to the local blood supply and local perfusion by removing cored tissue portions. In some embodiments, direct channels or holes may be used to deliver one or more useful therapeutic agents. Depending on the size (e.g. diameter and/or active length) of hollow needles, holes having different diameters and/or penetration depths may be created. For example, hollow needles having a large diameter (e.g. 18 gauge) and/or a long active length may be used to create large and/or deep holes that may be used as delivery channels to deliver a large volume dose of therapeutic agents. In some embodiments, holes may be plugged. In some embodiments, holes may be covered with a dressing (e.g. a compressive or occlusive dressing) and/or a closure (e.g. bandage, hemostats, sutures, or adhesives) to prevent the delivered therapeutic agents from leaking out of the skin and/or to maintain moisture of the treated skin area. Delivery of useful therapeutic agents through the holes created by the hollow needles of the apparatus may provide precise control of dosing of the therapeutic agents.

Examples of therapeutic agents that may be delivered using the technologies described herein include one or more growth factors (e.g. vascular endothelial growth factor (VEGF), platelet-derived growth factor (PDGF), transforming growth factor beta (TGF-b), fibroblast growth factor (FGF), epidermal growth factor (EGF), and/or keratinocyte growth factor); one or more stem cells (e.g. adipose tissue-derived stem cells and/or bone marrow-derived mesenchymal stem cells); one or more skin whitening agents (e.g. hydroquinone); one or more vitamin A derivatives (e.g. tretinoin), one or more analgesics (e.g. paracetamol/acetaminophen, aspirin, a non-steroidal anti-inflammatory drug, as described herein, a cyclooxygenase-2-specific inhibitor, as described herein, dextropropoxyphene, co-codamol, an opioid (e.g. morphine, codeine, oxycodone, hydrocodone, dihydromorphine, pethidine, buprenorphine, tramadol, or methadone), fentanyl, procaine, lidocaine, tetracaine, dibucaine, benzocaine, p-butylaminobenzoic acid 2-(diethylamino) ethyl ester HCl, mepivacaine, piperocaine, dyclonine, or venlafaxine); one or more antibiotics (e.g. cephalosporin, bactitracin, polymyxin B sulfate, neomycin, bismuth tribromophenate, or polysporin); one or more antifungals (e.g. nystatin); one or more antiinflammatory agents (e.g. a non-steroidal antiinflammatory drug (NSAID, e.g. ibuprofen, ketoprofen, flurbiprofen, piroxicam, indomethacin, diclofenac, sulindac, naproxen, aspirin, ketorolac, or tacrolimus), a cyclooxygenase-2-specific inhibitor (COX-2 inhibitor, e.g. rofecoxib (Vioxx®), etoricoxib, and celecoxib (Celebrex®)), a glucocorticoid agent, a specific cytokine directed at T lymphocyte function), a steroid (e.g. a corticosteroid, such as a glucocorticoid (e.g. aldosterone, beclometasone, betamethasone, cortisone, deoxycorticosterone acetate, dexamethasone, fludrocortisone acetate, hydrocortisone, methylprednisolone, prednisone, prednisolone, or triamcinolone) or a mineralocorticoid agent (e.g. aldosterone, corticosterone, or deoxycorticosterone)), or an immune selective antiinflammatory derivative (e.g. phenylalanine-glutamine-glycine (FEG) and its D-isomeric form (feG))); one or more antimicrobials (e.g. chlorhexidine gluconate, iodine (e.g. tincture of iodine, povidone-iodine, or Lugol's iodine), or silver, such as silver nitrate (e.g. as a 0.5% solution), silver sulfadiazine (e.g. as a cream), or Ag⁺ in one or more useful carriers (e.g. an alginate, such as Acticoat® including nanocrystalline silver coating in high density polyethylene, available from Smith & Nephew, London, U.K., or Silvered® including a mixture of alginate, carboxymethylcellulose, and silver coated nylon fibers, available from Systagenix, Gatwick, U.K.; a foam (e.g. Contreet® Foam including a soft hydrophilic polyurethane foam and silver, available from Coloplast A/S, Humlebsek, Denmark); a hydrocolloid (e.g. Aquacel® Ag including ionic silver and a hydrocolloid, available from Conva Tec Inc., Skillman, NJ); or a hydrogel (e.g. Silvasorb® including ionic silver, available from Medline Industries Inc., Mansfield, MA)); one or more antiseptics (e.g. an alcohol, such as ethanol (e.g. 60-90%), 1-propanol (e.g. 60-70%), as well as mixtures of 2-propanol/isopropanol; boric acid; calcium hypochlorite; hydrogen peroxide; manuka honey and/or methylglyoxal; a phenol (carbolic acid) compound, e.g. sodium 3,5-dibromo-4-hydroxybenzene sulfonate, trichlorophenylmethyl iodosalicyl, or triclosan; a polyhexanide compound, e.g. polyhexamethylene biguanide (PHMB); a quaternary ammonium compound, such as benzalkonium chloride (BAC), benzethonium chloride (BZT), cetyl trimethylammonium bromide (CTMB), cetylpyridinium chloride (CPC), chlorhexidine (e.g. chlorhexidine gluconate), or octenidine (e.g. octenidine dihydrochloride); sodium bicarbonate; sodium chloride; sodium hypochlorite (e.g. optionally in combination with boric acid in Dakin's solution); or a triarylmethane dye (e.g. Brilliant Green)); one or more antiproliferative agents (e.g. sirolimus, tacrolimus, zotarolimus, biolimus, or paclitaxel); one or more emollients; one or more hemostatic agents (e.g. collagen, such as microfibrillar collagen, chitosan, calcium-loaded zeolite, cellulose, anhydrous aluminum sulfate, silver nitrate, potassium alum, titanium oxide, fibrinogen, epinephrine, calcium alginate, poly-N-acetyl glucosamine, thrombin, coagulation factor(s) (e.g. II, V, VII, VIII, IX, X, XI, XIII, or Von Willebrand factor, as well as activated forms thereof), a procoagulant (e.g. propyl gallate), an anti-fibrinolytic agent (e.g. epsilon aminocaproic acid or tranexamic acid), and the like); one or more procoagulative agents (e.g. any hemostatic agent described herein, desmopressin, coagulation factor(s) (e.g. II, V, VII, VIII, IX, X, XI, XIII, or Von Willebrand factor, as well as activated forms thereof), procoagulants (e.g. propyl gallate), antifibrinolytics (e.g. epsilon aminocaproic acid), and the like); one or more anticoagulative agents (e.g. heparin or derivatives thereof, such as low molecular weight heparin, fondaparinux, or idraparinux; an anti-platelet agent, such as aspirin, dipyridamole, ticlopidine, clopidogrel, or prasugrel; a factor Xa inhibitor, such as a direct factor Xa inhibitor, e.g. apixaban or rivaroxaban; a thrombin inhibitor, such as a direct thrombin inhibitor, e.g. argatroban, bivalirudin, dabigatran, hirudin, lepirudin, or ximelagatran; or a coumarin derivative or vitamin K antagonist, such as warfarin (coumadin), acenocoumarol, atromentin, phenindione, or phenprocoumon); one or more immune modulators, including corticosteroids and non-steroidal immune modulators (e.g. NSAIDS, such as any described herein); one or more proteins; and/or one or more vitamins (e.g. vitamin A, C, and/or E). One or more of botulinum toxin, fat (e.g. autologous), hyaluronic acid, a collagen-based filler, or other filler may also be administered to the skin. Platelet rich plasma may also be administered to the skin. One or more therapeutic agents described herein may be formulated as a depot preparation. In general, depot preparations are typically longer acting than non-depot preparations. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a therapeutic agent may include anticoagulative and/or procoagulative agents. For instance, by controlling the extent of bleeding and/or clotting in treated skin regions, a skin tightening effect may be more effectively controlled. Thus, in some embodiments, the methods and devices herein include or can be used to administer one or more anticoagulative agents, one or more procoagulative agents, one or more hemostatic agents, one or more fillers, and/or combinations thereof. In particular embodiments, the therapeutic agent controls the extent of bleeding and/or clotting in the treated skin region, including the use one or more anticoagulative agents (e.g. to inhibit clot formation prior to skin healing or slit/hole closure) and/or one or more hemostatic or procoagulative agents.

Components of different embodiments described in this specification may be combined to form other embodiments not specifically set forth in this specification. Components may be left out of the systems, apparatuses, etc. described in this specification without adversely affecting their operation.

In addition, the logic flows shown in, or implied by, the figures do not require the particular order shown, or sequential order, to achieve desirable results. Various separate components may be combined into one or more individual components to perform the functions described here.

### EXAMPLE EMBODIMENTS

Embodiment 1: An apparatus for producing a cosmetic effect in skin tissue, the apparatus comprising:
(i) a needle hub comprising at least one hollow needle having a distal end for contacting skin and configured to remove a portion of the skin tissue (e.g. a microcore) when the hollow needle is inserted into and withdrawn from the skin tissue;
(ii) a translation and/or actuation mechanism connected to the needle hub to translate and/or actuate the needle hub in one or more directions relative to a surface of the skin tissue; and
(iii) a spacer to stabilize and/or maintain a constant position of the apparatus relative to the surface of the skin tissue.

Embodiment 2: The apparatus of Embodiment 1, comprising a hand piece shell at least partially enclosing the translation and/or actuation mechanism.

Embodiment 3: The apparatus of Embodiment 1 or Embodiment 2, wherein the spacer is attached to the hand piece shell.

Embodiment 4: The apparatus of any of Embodiments 1-3, wherein the needle hub comprises a single hollow needle.

Embodiment 5: The apparatus of any of Embodiments 1-4, wherein the needle hub comprises three hollow needles arranged in a row.

Embodiment 6: The apparatus of any of Embodiments 1-5, wherein the needle hub comprises a two-dimensional array of needles (e.g. a two-by-two, three-by-two, or three-by-three array).

Embodiment 7: The apparatus of any of Embodiments 1-6, wherein the needle hub comprises a first lumen having a first end and a second end, wherein the first lumen comprises a lumen of the at least one hollow needle and wherein the first end of the first lumen is at the distal end of the hollow needle.

Embodiment 8: The apparatus of any of Embodiments 1-7, wherein the needle hub comprises a second lumen having a wall, a first end, and a second end, wherein the first end of the second lumen is or comprises a fluid intake nozzle.

Embodiment 9: The apparatus of any of Embodiments 1-8, wherein the first lumen is connected to the second lumen such that the second end of the first lumen forms an opening in the wall of the second lumen.

Embodiment 10: The apparatus of any of Embodiments 1-9, wherein each of the first lumen and the second lumen are substantially straight, and wherein the first lumen is substantially perpendicular to the second lumen forming a T-junction.

Embodiment 11: The apparatus of any of Embodiments 1-10, wherein the fluid intake nozzle is a convergent nozzle.

Embodiment 12: The apparatus of any of Embodiments 1-11, wherein the second end of the second lumen is connected to a fluid conduit such that when low pressure or vacuum is applied to the conduit, low pressure or vacuum is induced in the first lumen and the second lumen, such that fluid is drawn into and through the second lumen through the first end of the second lumen, thereby clearing skin tissue from the first lumen.

Embodiment 13: The apparatus of any of Embodiments 1-12, wherein the translation and/or actuation mechanism comprises an actuator to displace the needle hub along a z-axis in a direction substantially perpendicular to a surface of the skin tissue and substantially parallel to a longitudinal axis of the at least one hollow needle.

Embodiment 14: The apparatus of any of Embodiments 1-13, wherein the actuator is or comprises a voice coil.

Embodiment 15: The apparatus of any of Embodiments 1-14, comprising a sensing device for detecting a position of the needle hub along the z-axis.

Embodiment 16: The apparatus of any of Embodiments 1-15, wherein the translation and/or actuation mechanism comprises an x/y-stage to translate the needle hub in one or more directions parallel to the surface of the skin.

Embodiment 17: The apparatus of any of Embodiments 1-16, wherein the translation and/or actuation mechanism comprises a rotary stage to rotate the needle hub around the z-axis.

Embodiment 18: The apparatus of any of Embodiments 1-17, wherein the spacer comprises a device to contact a surface of the skin tissue, and to (a) to maintain a distance and/or position between the apparatus and the skin tissue and/or (b) maintain or increase tension in the skin tissue during treatment compared to the skin tissue not being treated and/or contacted by an apparatus.

Embodiment 19: The apparatus of any of Embodiments 1-18, wherein the spacer comprises a frame to contact the surface of the skin tissue, wherein the frame comprises a base, an inner wall, and an outer wall, wherein the base, inner wall, and outer wall form an open channel.

Embodiment 20: The apparatus of any of Embodiments 1-19, wherein the channel is configured such that when the frame is placed on the surface of the skin, the surface of the skin, the base, the inner wall, and outer wall form a frame lumen.

Embodiment 21: The apparatus of any of Embodiments 1-20, wherein the frame is connected to a fluid conduit such that when low pressure or vacuum is applied to the conduit, low pressure or vacuum is established in the frame lumen, thereby drawing skin tissue toward and/or into the channel.

Embodiment 22: The apparatus of any of Embodiments 1-21, wherein the base comprises one or more protrusions.

Embodiment 23: The apparatus of any of Embodiments 1-22, wherein the frame is contoured (e.g, wherein the frame is concave).

Embodiment 24: The apparatus of any of Embodiments 1-23, wherein the spacer comprises a switch connected to a sensor to detect a position of the apparatus relative to tissue underlying the skin, wherein
(a) when the frame is placed on the surface of the skin and a low pressure or vacuum is applied to the frame, the switch is in a “no-go” position, and
(b) when the frame, while the frame is in contact with the surface of the skin after a low pressure or vacuum is applied to the frame, and after the frame is moved in a direction that is substantially perpendicular to and away from the surface of the skin, the switch is in a “go” position;
wherein, when the switch is in the no-go position, the needle hub is prevented from moving along a z-axis in a direction substantially perpendicular to a surface of the skin tissue and substantially parallel to a longitudinal axis of the at least one hollow needle; and wherein, when the switch is in the go position, the needle hub is moveable along the z-axis.

Embodiment 25: The apparatus of any of Embodiments 1-24, wherein the sensor is or comprises a pushrod.

Embodiment 26: A system comprising the apparatus of any of Embodiments 1-25, the system comprising a removal system for removing one or more tissue portions from the apparatus.

Embodiment 27: The system of Embodiment 26, wherein the removal system comprises a low-pressure source (e.g. a vacuum pump).

Embodiment 28: The system of any of Embodiments 26-27, wherein the low-pressure source is connected to the needle hub comprising the at least one hollow needle via a first conduit to provide suction in the at least one hollow needle.

Embodiment 29: The system of any of Embodiments 26-28, wherein the low-pressure source is connected to the spacer via a second conduit to provide suction in the spacer.

Embodiment 30: The apparatus of any of Embodiments 1-24, wherein the at least one hollow needle comprises at least a first prong provided at a distal end of the hollow needle for contacting skin, wherein an angle between a lateral side of the first prong and a longitudinal axis of the hollow needle is at least about 20 degrees.

Embodiment 31: The apparatus of any of Embodiments 1-24, wherein the at least one hollow needle comprises a second prong at the distal end of the hollow needle.

Embodiment 32: The apparatus of any of Embodiments 1-24, wherein the first prong and/or the second prong comprises a flat tip.

Embodiment 33: The apparatus of any of Embodiments 1-24, wherein the first prong and/or the second prong comprises an edge.

Embodiment 34: The apparatus of any of Embodiments 1-24, wherein an inner diameter of the at least one hollow needle is between about 0.14 mm and 0.84 mm.

Embodiment 35: The apparatus of any of Embodiments 1-24, wherein an inner diameter of the at least one hollow needle is between about 0.24 mm and 0.40 mm.

Embodiment 36: The apparatus of any of Embodiments 1-24, wherein the at least one hollow needle is configured to extend (i) into the dermal layer, (ii) through the entire dermal layer to the junction of the dermal layer and the subcutaneous fat layer, or (iii) into the subcutaneous fat layer.

Embodiment 37: An apparatus comprising a hollow needle and a pushrod moveably disposed therein.

Elements of different embodiments described herein may be combined to form other embodiments not specifically set forth above. Elements may be left out of the technologies, systems, apparatuses computer programs, user interfaces, etc. described herein without adversely affecting their operation or the operation of the technologies in general. Furthermore, various separate elements may be combined into one or more individual elements to perform the functions described herein.

The foregoing description of various embodiments has been presented for purposes of illustration and description. The foregoing description is not intended to limit the claims to the embodiment disclosed herein.

Referring now to FIG. 35, a circuit schematic of a tissue treatment system is illustrated, consistent with the present inventive concepts. In FIG. 35, electrical connections (including single conductors and multi-conductor buses) are shown with thin lines, and vacuum connections are shown with thick lines. System 10 can include one or more of the various components illustrated in FIG. 35. In FIG. 35, dashed lines are used to define a particular arrangement of components as they can be integrated into treatment device 100 and console 500. FIG. 35 illustrates various components of system 10 (e.g. as described herein) in a particular arrangement. Console 500 and/or treatment device 100 (“devices 500/100” herein) can comprise a vacuum pump (e.g. as shown as part of console 500), such as a functional element 199 and/or 599 configured as a vacuum pump (e.g. a vacuum pump comprising an adjustable pressure regulator). Devices 500/100 can comprise one or more fans (e.g. as shown as part of console 500) which can be configured to cool one or more portion of devices 500/100, such as to cool a pump (e.g. a vacuum pump) of a device 500/100.

Console 500 comprises electronic circuitry to provide a drive signal (e.g. voltage and current) to z-actuator 121z (e.g. a z-actuator 121z including a voice coil). The drive signal can comprise a differential signal for current and voltage. The drive signal electronic circuitry can comprise a temperature sensor (e.g. a sensor 599a configured as a temperature sensor), such as to confirm proper operation of this circuitry. Console 500 can comprise various sensors (e.g. one or more sensors 599a), such as sensors selected from the group consisting of: temperature sensors; pressure sensors such as vacuum sensors (e.g. to monitor vacuum provided by console 500); position sensors such as a ratiometric hall sensor or other position sensor; voltage sensors; current sensors; and combinations of these. In some embodiments, one or more sensors of system 10 (e.g. sensor 199a of treatment device 100) is used to confirm proper attachment of treatment module 150 to treatment device 100. In some embodiments, one or more sensors of system 10 (e.g. sensor 599a of console 500) is used to confirm proper attachment of treatment device 100 to console 500. In some embodiments, a vacuum line of treatment module 150 attaches directly to console 500 without passing through other portions of treatment device 100.

In some embodiments, treatment device 100 does not include a pressure sensor, however console 500 does include a pressure sensor (e.g. a sensor 599a comprising a pressure sensor) that measures pressure at an output of console 500 that is proximate a fluidic attachment of console 500 to a fluidic pathway of treatment device 100. In these embodiments, the fluidic pathway of treatment device 100 that attaches to console 500 is directly connected to a fluidic pathway of treatment module 150 (e.g. there is a relatively small distance between the treatment module 150 pathway and the associated pressure sensor of console 500).

System 10 can comprise a communication line (e.g. a 10 MHz LVDS communication line) configured to transfer information between console 500 and treatment device 100.

System 10 can comprise one or more memory storage components (e.g. memory 522 described herein), such as system parameter information installed during manufacturing of system 10 and/or user input parameter information. In some embodiments, calibration data is stored (e.g. in memory 522) by system 10. In some embodiments, memory (e.g. memory 522) stores instructions (e.g. instructions 523 described herein) to perform one or more algorithms (algorithm 525 described herein), such as one or more AI algorithms.

Treatment device 100 can include a unique identifier (e.g. provided via an RFID chip as described herein), and console 500 can comprise a reader (e.g. RFID reading device). In some embodiments, treatment device 100 comprises a kit of multiple treatment modules 150, where each treatment module 150 comprises a unique identifier (e.g. an RFID chip), such as an identifier that includes a unique serial or lot number for the module 150, and/or manufacturing information of module 150 (e.g. coring element 155 information such as number of elements 155 per module, element 155 calibrations parameters, and the like).

System 10 can include one or more field programmable gate arrays (FPGAs). For example, FPGAs can be configured to provide LVDS serial communication between treatment device 100 and console 500, such as communication provided in full duplex mode (e.g. to monitor and capture data in near real time, “real time” herein). In some embodiments, treatment device 100 can comprise a single FPGA or other “single” component that is configured to provide: control of one or more motors; control of one or more indicating lights (e.g. one or more LEDs); interface with one or more sensors; and/or communication between two or more components of the system (e.g. between treatment device 100 and console 500), such as a single FPGA or other single component that is configured to provide, one, two, three, and/or all four of these. In some embodiments, the implementation of a single FPGA configured to control multiple components allows for a simplified control algorithm (e.g. algorithm 525), such as an algorithm that does not handle communication between multiple FPGAs.

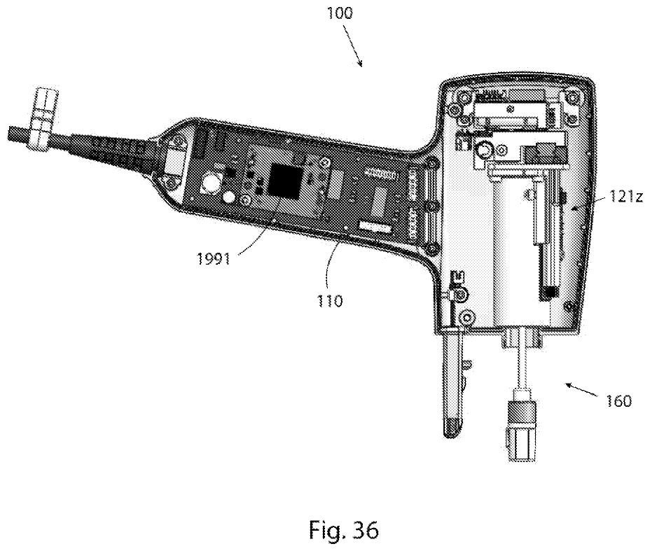

Referring now to FIG. 36, a side view of a treatment device with a portion of a housing removed is illustrated, consistent with the present inventive concepts. Treatment device 100 can include one or more of the various components illustrated in FIG. 36. For example, treatment device 100 can comprise a temperature sensor, such as sensor 1991 shown. Sensor 1991 can comprise one or more thermistors, thermocouples, and/or other temperature sensors. System 10 can be configured to enter an alert state if temperature recorded by sensor 1991 (e.g. a temperature of a housing and/or a component within a housing, such as a voice coil) is outside of an accepted temperature range (e.g. to prevent burning or undesired temperature rise to a clinician operator and/or the patient). In some embodiments, sensor 1991 is positioned proximate one or more particular components whose temperature is to be monitored, such as an actuator 121 (e.g. a voice coil actuator), a portion of the housing (e.g. housing 110), and/or other component of treatment device 100.

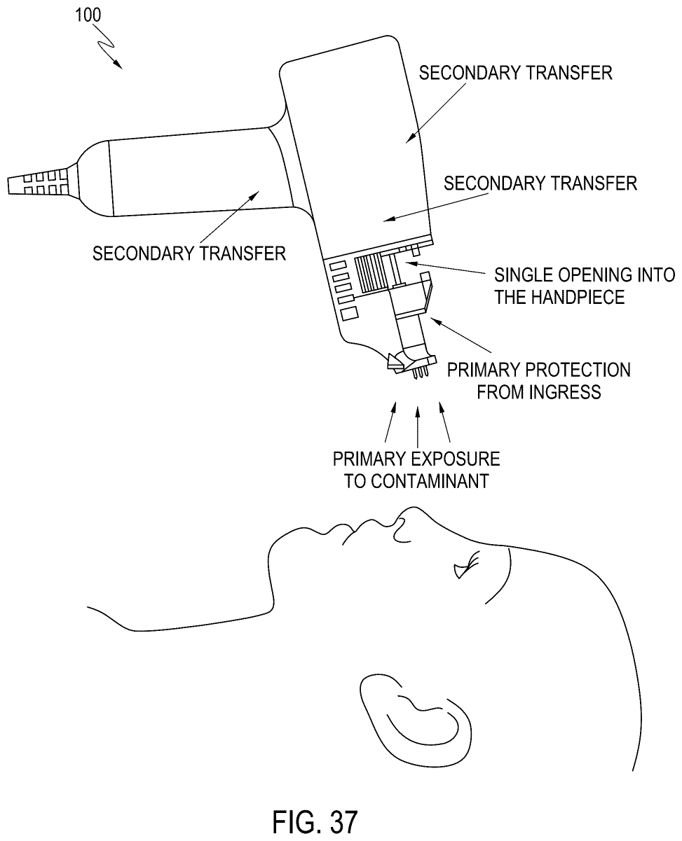

Referring now to FIG. 37, a side view of a treatment device positioned relative to a patient's face is illustrated, consistent with the present inventive concepts. Treatment device 100 can include one or more of the various components illustrated in FIG. 37. In some embodiments, treatment device 100 includes an arrangement of one or more housings (e.g. including a handle portion as shown) that are constructed and arranged such that only a single opening (e.g. as shown) is present between the outer surface of device 100 and its internal components (e.g. to limit potential contaminants passing into locations containing the internal components of device 100). Locations of primary and secondary contaminant transfer are also shown in the FIG. 37. System 10 can include a cap (not shown) to be inserted into the single opening during cleaning of treatment device 100.

Referring now to FIGS. 38A-C, a perspective view, a side view, and a perspective view, respectively, of a distal portion of a treatment device are illustrated, consistent with the present inventive concepts. Treatment device 100 can include a cover, shroud 151 shown, which is positioned to limit ingress of material (e.g. blood or other tissue) into an opening of device 100 (e.g. the single opening described in reference to FIG. 37 herein). In some embodiments, presence of shroud 151 avoids the need for a separate component (e.g. a drape) being positioned on and/or about device 100 during use, such as to avoid any material or component that might impede x, y, and/or z motion of treatment module 150 during a microcoring procedure. Shroud 151 can be configured to be disposed of after each clinical use of device 100.

Treatment device 100 can include a frame, flange 152 shown, which can be positioned on the patient's skin prior to and during a microcoring procedure. Flange 152 can be configured to provide suction (e.g. as described herein), such as to provide a retaining force of device 100 to the patient's skin during treatment while vacuum is applied (e.g. via console 500) to flange 152. Treatment device 100 can include a mechanical stop, surface 153 which prevents advancement of treatment module 150 from exceeding a distance beyond flange 152 (e.g. from advancing more than 6.75 mm beyond flange 152). Suction provided by flange 152 can cause the tissue surface surrounded by flange 152 to be kept relatively taught, such as to reduce penetration force of microcoring elements 155 into tissue.

Treatment module 150 (e.g. its housing, and flange 152) comprises a geometry that is configured to prevent a coring element 155 from inadvertently penetrating the fingers or other tissue portion of an operator (e.g. a clinician) using treatment device 100.

System 10 can be configured such that the one or more microcoring elements 155 (three shown in FIG. 38A) of an attached treatment module 150 are positioned in a “corner” of flange 152 (e.g. at maximum and/or minimum x and y positions) at the time that the operator (e.g. a clinician) is positioning flange 152, for example such that a view of the operator to the skin surface within flange 152 is maximized. Alternatively or additionally, system 10 can be configured such that the one or more microcoring elements 155 of an attached treatment module 150 are positioned at an x-y location that will be used (e.g. without subsequent x-y motion) for the first advancement (e.g. z-advancement) of elements 155 into tissue, for example such that the operator knows the first tissue portion to be punctured.

As shown in FIG. 38B, treatment device 100 can include Y-connector 154 which is configured to provide a vacuum to the lumen of coring elements 155 via tube 1541 such as to remove tissue from elements 155, as well as to provide a vacuum to flange 152 via tube 1542. Y-connector 154 comprises angle A1 between tubes 1541 and 1542. Angle A1 comprises an angle less than 90°, such as an angle less than 75°, less than 45°, and/or less than 30°, such as to create a vacuum flow pathway 1545 that is resistant to clogging (e.g. captured tissue avoids having to pass through large bends). Vacuum flow pathway 1545 has a proximal portion at each lumen of a coring element 155. Traveling distally, pathway 1545 passes through Y-connector 154 and travels further distally into fluidly connected tubing of console 500. In some embodiments, vacuum flow pathway 1545 never decreases in diameter in its proximal to distal direction. For example, the pathway 1545 can increase in diameter in the proximal to distal direction, either continuously or in discrete steps, such as to avoid clogging. Y-connector 154, and one or more other portions of flow pathway 1545, can be relatively fixed to a housing of treatment device 100 (e.g. Y-connector 154 can be attached to a housing of treatment module 150, such as to avoid undesired forces being applied to treatment module 150 that might affect x, y, and/or z positions of coring elements 155 during a microcoring procedure).

FIG. 38C shows a treatment module 150 prior to its attachment to a receiving portion 160 which includes projection 161, each as shown. Projection 161 is configured to slidingly engage a receiving portion, port 156 of treatment module 150 (hidden from view in FIG. 38C). Treatment module 150 can comprise a single component, such as a single component that is attached by an operator to receiving portion 160 in a single step. Treatment module 150 can be configured such that coring elements 155 are in a locked state, such as a state in which elements 155 are locked in place when not attached to receiving portion 160 (e.g. while stored, during shipment, during de-packaging such as during removal from a tray, and during attachment to receiving portion 160 of device 100). Upon attachment of treatment module 150 to receiving portion 160, coring elements 155 can be moved (e.g. in x, y, and/or z-directions by actuators 121 of device 100). For example, treatment module 150 can comprise a sliding locking mechanism (e.g. a locking mechanism comprising a set of one or more hooks that engage and prevent motion of elements 155 during storage and other times not attached to receiving portion 160), and projection 161 can be configured to, during attachment of module 150, cause a portion of the locking mechanism (e.g. the set of one or more hooks) to move to slidingly disengage, allowing motion of elements 155. Treatment module 150 and/or another portion of treatment device 100 can comprise a sensor assembly (e.g. sensor 199a described herein), that is constructed and arranged to provide a signal indicative of the proper attachment of treatment module 150 to receiving portion 160, and/or a signal indicative of the locked or unlocked status of elements 155. For example, sensor 199a can comprise a proximity sensor assembly configured to detect the proper attachment of treatment module 150 to receiving portion 160. In some embodiments, sensor 199a comprises a first component 199a-1 (not shown) comprising a magnet or magnetic sensor positioned in receiving portion 160, and s second component 199a-2 (shown in FIG. 38a) comprising a corresponding magnetic sensor (e.g. a Hall effect sensor) or magnet, respectively, positioned in treatment module 150. As described herein, treatment module 150 can comprise a unique identifier and/or memory component (e.g. an RFID) that includes serial and/or lot number information, calibration data, and the like.

FIG. 38D is a photograph of a side view of a distal portion of a treatment device, and FIGS. 38E-F are side sectional views of a distal portion of a treatment device, consistent with the present inventive concepts. The distal portion of treatment device 100 is shown with an attached treatment module 150.

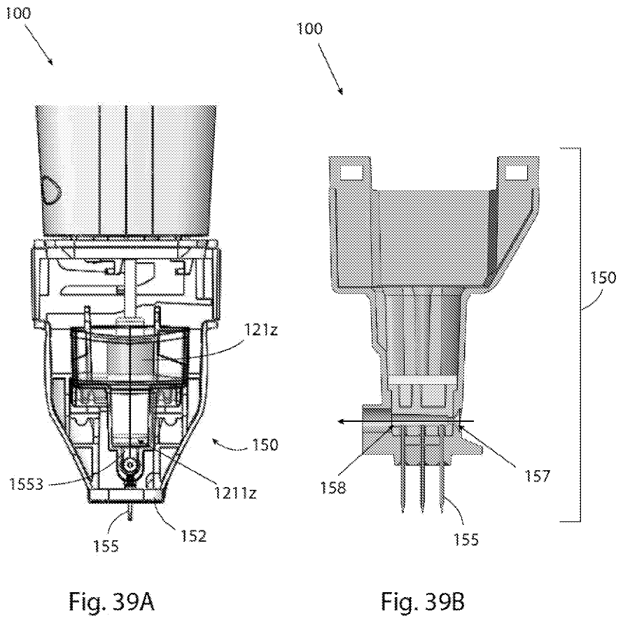

Referring now to FIGS. 39A and 39B, a partially transparent and a cross-sectional side view of a distal portion of a treatment device, respectively, are illustrated, consistent with the present inventive concepts. Treatment device 100 of FIGS. 39A-B includes a treatment module 150 comprising three microcoring elements 155 and flange 152. As described herein, flange 152 can be attached to a vacuum line (e.g. lumens of tubes) such that flange 152 can grip a skin surface via suction. Vacuum, via one or more vacuum lines (e.g. including pathway 158 shown) can also be applied to lumens of elements 155 to clear tissue cores present within elements 155, also as described herein. In some embodiments, treatment device 100 is configured to provide a “metered leak”, such as a leak pathway provided via hole 157 shown. Hole 157 can comprise a hole with a diameter of at least 0.2 mm, such as at least 0.4 mm, 0.6 mm, and/or 0.8 mm, such as a hole with a diameter of approximately 1.1 mm. Hole 157 can comprise a hole with a diameter of no more than 3 mm, such as no more than 2.5 mm, 2.0 mm, and/or 1.5 mm, such as a hole with a diameter of approximately 1.1 mm. Hole 157 can comprise a hole with a taper, such as a taper configured to provide a consistent vacuum at the lumens of each of the elements 155. For example, hole 157 can comprise a taper of at least 1 degree, and/or a taper of no more than 5 degrees, such as a taper of approximately 2 degrees. Hole 157 can be constructed and arranged (e.g. positioned and sized) to provide a particular level of vacuum at flange 152 (e.g. vacuum at a level of at least 30 kPa, or at least 40 kPa, or between 40 kPa and 50 kPa), while maintaining a minimum flow velocity at the lumens of elements 155 (e.g. a flow velocity of at least 1 m/sec, such as at least 1.5 m/sec, and/or approximately 2 m/sec). Treatment module 150 comprises a hub 1553 as shown, which is attached to coring elements 155. In some embodiments, z-actuator 121z can comprise a distal portion 1211z that comprises a magnet or magnetic material, and hub 1553 comprises a mating magnet and/or magnetic material, such that a magnetic attraction is created between z-actuator 121z and hub 1553.

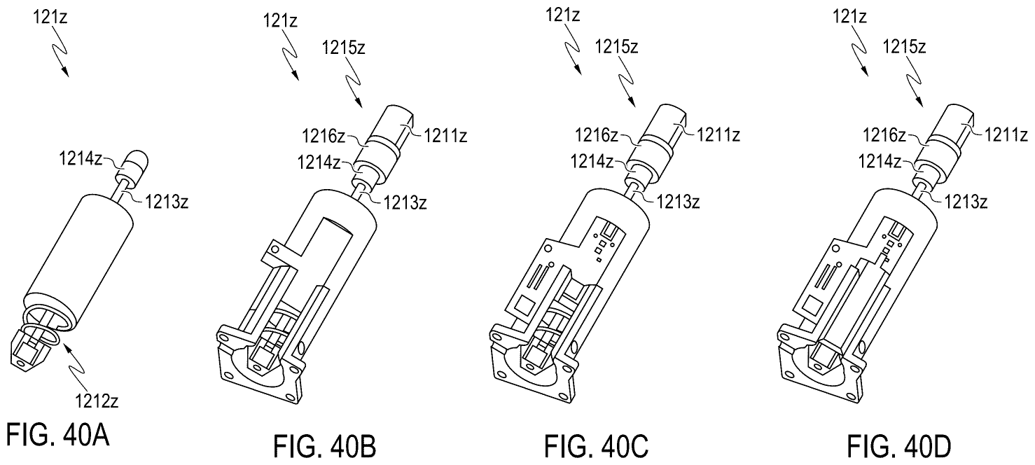

Referring now to FIGS. 40A-D, a side view and three sectional side views of a z-actuator of a treatment device are illustrated, consistent with the present inventive concepts. Z-actuator 121-z can be configured to be translated in x and/or y directions, as described herein. As shown in FIGS. 40A-D, z-actuator 121z can comprise one or more springs, such as spring 1212z. Spring 1212z can be configured to maintain the coring elements 155 of an attached treatment module 150 in a retracted position when treatment device 100 is in an unpowered state. Z-actuator 121z can comprise a translatable shaft, shaft 1213z. Shaft 1213z is configured to translate in a reciprocating manner in the z direction (e.g. via a voice coil or other linear actuator of z-actuator 121z as described herein). Attached to shaft 1213z is adaptor 1214z (e.g. a threaded adaptor). Attached (e.g. threadedly attached) to adaptor 1214z is assembly 1215z comprising insulator 1216z, and distal portion 1211z (e.g. distal portion 1211z described in reference to FIG. 39A herein, such as a magnet and/or magnetic material configured to magnetically couple with a mating magnetic component of treatment module 150, also as described herein). Assembly 1215z is configured to operably attach to various configurations of treatment module 150, such as to operably attach to one, two, or more treatment modules 150 with different quantities, different types (e.g. different diameters and/or different lengths), and/or different geometric arrangements of microcoring elements 155. In some embodiments, multiple assemblies 1215z are included (e.g. and each attachable to adaptor 1214z), wherein each assembly 1215z is configured to attach to one or more different configurations of treatment module 150. Insulator 1216z can be configured to electrically isolate any electrical signals, connections, conduits, and/or other energy-carrying means that are present within device 100 from the patient.

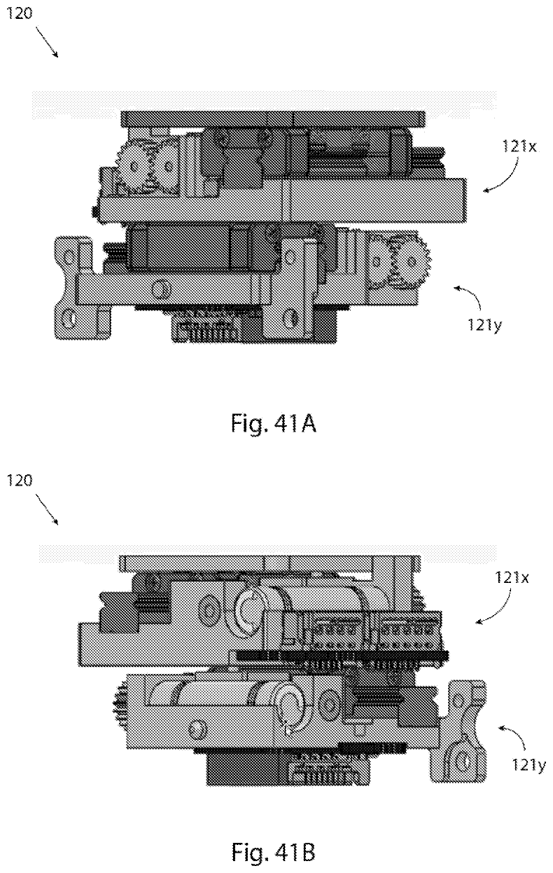

Referring now to FIGS. 41A-B, two side views of an actuation assembly of a treatment device are illustrated, consistent with the present inventive concepts. Actuation assembly 120 of FIGS. 41A-B includes actuators 121x and 121y, which can be configured to translate an actuator 121z in x and/or y directions, such as is described herein. Actuators 121x and 121y can be of similar construction and arrangement as the similar components described in applicant's co-pending U.S. patent application Ser. No. 17/291,235, titled “Systems and Methods for Skin Treatment”, May 4, 2021. In some embodiments, actuators 121x and/or 121y are configured to provide a translational accuracy of +no more than 0.100 mm, such as no more than 0.080 mm, no more than 0.060 mm, such as approximately 0.042 mm. In some embodiments, the translation step increment is chosen to provide numerous microcoring patterns. The step increment can be above a minimum, such as a minimum that avoids cutting and/or tearing between adjacent coring locations. Actuators 121x and 121y can be arranged in various arrangement geometries relative to each other. In some embodiments, an actuator 121x is positioned in a first orientation, and an actuator 121y is positioned such that its actuation is orthogonal to the actuation of actuator 121x, such as to collectively provide motion of a component (e.g. actuator 121z as described herein) in two orthogonal directions (x and y), in various translation increments as described above. Each actuator 121x and 121y can include a motor, gears, a leadscrew, a threaded block, rotational bearings, and/or lateral bearings, such as to translate bi-directional rotational movement into bi-directional lateral movement. Each actuator 121x and 121y can include one or more position encoders (e.g. one or more rotational encoders and/or linear encoders), as well as marker elements and/or sensors that are singly and/or collectively configured to provide motion and/or position information, such that the provided two directional motion can achieve sufficient accuracy and/or be operated in a closed loop mode. For example, algorithm 525 described herein can utilize the various forms of motion and/or position information to create motion commands (e.g. velocity and/or acceleration commands) and/or position commands that cause the motors of the actuators 121x and/or 121y to achieve specific positions (e.g. achieve the specific positions with a target speed and/or acceleration). The actuators 121x and 121y can be collectively configured to allow x-y control of an attached assembly of one or more coring elements 155 (e.g. elements 155 being attached to an actuator 121z which is attached to actuators 121x and 121y) such that the coring elements can travel a distance of at least 8 mm, at least 12 mm, at least 16 mm, as least 20 mm, at least 30 mm, and/or at least 40 mm in both x and y directions.

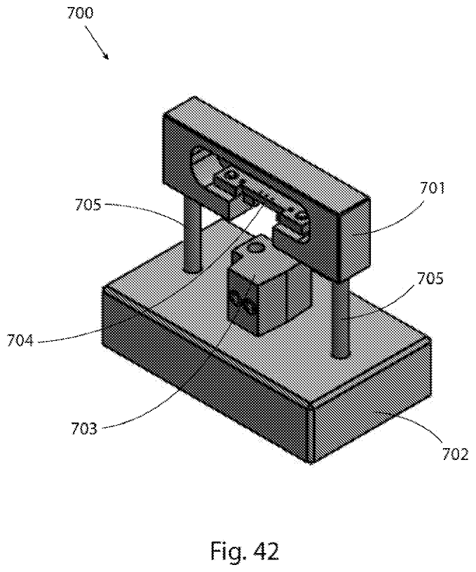

Referring now to FIG. 42, a perspective view of a tool for manufacturing a portion of a tissue treatment system is illustrated, consistent with the present inventive concepts. Manufacturing tool 700 shown in FIG. 42 comprises a tool configured to manufacture at least a portion of a treatment module 150 of the present inventive concepts, such as to fixedly attach, in a desired geometry, one or more (e.g. three) coring elements 155 to hub 159 (not shown), a receiving portion of a treatment module 150. For example, hub 159 can comprise multiple receiving holes 1591 (also not shown), and each receiving hole 1591 can be sized to slidingly receive and be adhesively attached to a proximal portion of a coring element 155. Tool 700 of FIG. 42 includes base block 702 which includes projection 703 as shown. Projection 703 can be configured to removably attach to hub 159 of a treatment module 150. Tool 700 further includes top block 701 which includes an aligning element, aligner 704 as shown, which can be configured to slidingly receive and align (e.g. axially and/or rotationally align) the distal portion of one or more coring elements 155, such as for subsequent attachment of the proximal portion of each element 155 to the hub 159 of a treatment module 150. Top block 701 is slidingly attached to base block 702 via posts 705, also as shown.

In some embodiments, aligner 704 slidingly receives the distal portion of one or more coring elements 155 (not shown, but such as three elements 155), such as without damaging the distal portion of each element 155 (e.g. without damaging a beveled distal end of each element 155). This alignment is made relative to hub 159 of a treatment module 150, during attachment of elements 155 to the hub 159 using tool 700. Aligner 704 can be constructed and arranged to axially position the exposed length of each element 155 extending from a surface of hub 159 after attachment of each element 155 to hub 159 (e.g. by positioning the distal end of each element 155 relative to the hub 159 surface). Alternatively and/or additionally, aligner 704 can be constructed and arranged to rotationally align the distal tip of each element 155. For example, aligner 704 can be configured to rotationally align a single or double beveled end (e.g. as described herein) of each element 155 relative to hub 159, and/or to a neighboring element 155 that is also attached to hub 159. This rotational alignment of each element 155 can be performed to avoid a linear alignment of multiple neighboring beveled tips (e.g. single or double beveled distal tip), such as to avoid a linear alignment of beveled tips which may tend to cause slicing of the skin surface during advancement and/or retraction of elements 155 through the skin surface. In some embodiments, tool 700, via aligner 704, angularly positions the beveled distal tips of neighboring elements 155 such that the axes of multiple element 155 beveled ends of an assembled treatment module 150 are in a relatively parallel arrangement.

Axial and/or rotational positioning of the distal end (e.g. the beveled end) of each element 155 can be achieved using a surface of aligner 704 which mates with the element 155 distal end (e.g. a single or double beveled distal end as described herein). This referencing allows precise control of the element 155 distal end position and angular rotation, such as while eliminating undesired effects that otherwise would result from manufacturing variability in lengths of elements 155.

During the manufacturing of treatment module 150, block 701, along with aligner 704 and inserted elements 155, is lowered to a surface (e.g. dead stop) on the hub 159 being attached to the elements 155, so that the tip position of each element 155 is located at a desired distance relative to this surface of hub 159. As described hereinabove, a desired angular arrangement of each distal end (e.g. beveled edge) of elements 155 can also be achieved. In a first manufacturing step, coring elements 155 are loaded into aligner 704 such that a receiving geometry of aligner 704 “funnels” (e.g. automatically positions and orients) the distal end of each element 155 into a desired position (e.g. the desired axial position, angular position, or both). Tool 700 can include one or more magnets (not shown, but such as three magnets, one for each element 155). These magnets can hold each element 155 in place (e.g. within aligner 704) during assembly (e.g. allowing elements 155 to be in a suspended arrangement in aligner 704, prior to insertion of elements 155 into receiving holes 1591 of hub 159). In a second manufacturing step (e.g. performed prior to or after the first manufacturing step), hub 159 is seated on projection 703 of base block 702. Base block 702 is desirably oriented with top block 701 and included aligner 704, via posts 705, such as to align the inserted elements 155 with the associated holes in the receiving hub. In a third manufacturing step, top block 701 is translated toward base block 702 (and/or vice versa), such that the proximal ends of coring elements 155 are slidingly positioned within holes 1591 of hub 159. Tool 700 can be configured such that adhesive can be dispensed (e.g. and cured) to bond the coring elements 155 to holes 1591. After bonding is complete (e.g. when the adhesive is cured), top block 701, including insert 704, can be translated away from base block 702, such that treatment module 150 (e.g. hub 159 including bonded elements 155) can be removed from projection 703 of base block 702.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1.-77. (canceled)

78. A system for producing a cosmetic effect in skin tissue of a patient, the system comprising:
a treatment module comprising at least one coring element configured to remove a portion of skin tissue when the coring element is inserted into and withdrawn from the skin tissue; and
an actuation assembly operably attached to the treatment module and configured to translate the treatment module in one or more directions relative to a surface of the skin tissue,
wherein the actuation assembly comprises a z-actuator that is configured to advance the at least one coring element into the skin,
wherein the system is configured to perform a microcoring procedure that provides a cosmetic effect to the patient, and
wherein the system includes a limit on depth of travel of the z-actuator to limit depth of penetration of the at least one coring element into the skin.

79. The system of claim 78, wherein the limit on depth of travel is a limit depth of travel of a translating component of the z-actuator that translates along a z-axis.

80. The system of claim 79, further comprising a console that comprises a user interface configured to input the depth of penetration by an operator.

81. The system of claim 79, wherein the depth of penetration is 3 mm, 4 mm, or 5 mm.

82. The system of claim 78, wherein the system is configured so that a titration or other iterative adjustment procedure is performed by an operator, in which the depth of penetration is adjusted.

83. The system of claim 78, wherein the system is configured to decelerate the z-actuator as the at least one coring element is approaching a target depth.

84. The system of any of claim 83, wherein the system is configured to monitor deceleration of a translating component of the z-actuator by monitoring the deceleration of the associated coring element and to adjust the depth of penetration.

85. The system of claim 84, wherein the system is configured to monitor the deceleration of the translating component of the z-actuator, and, if the monitored deceleration exceeds a maximum threshold (D_{MAX}) to enter an alarm or other alert state.

86. The system of claim 85, wherein the system has multiple deceleration thresholds comprising the maximum threshold (D_{MAX}) and at least one lower threshold (D₁, D₂),
wherein the system comprises an algorithm that is configured to record a number of times the z-actuator exceeds either or both thresholds, and to enter an alert state in which use of the system is stopped until further action of an operator is performed, or to enter an alert state in which use of the system can continue, but the operator is notified of the exceeding of the threshold.

87. The system of claim 78, further comprising one or more algorithms configured to control the z-actuator via one or more sets of instructions.

88. The system of claim 79, wherein ranges of positions of the translating component of the z-actuator range from a −zₘₐₓ to zₘₐₓ, with a rise time, tᵣ,
wherein the translating component of the actuator has a velocity, v, and a maximum velocity, vₘₐₓ, and an acceleration, a, and
wherein the acceleration a of the translating component of the actuator is controlled to approximate a smooth continuous function.

89. The system of claim 78, further comprising at least one treatment device, wherein the at least one treatment device is void of any surface, projection, or other mechanical stop that is contacted by the treatment module during advancement of the at least one coring element in a microcoring procedure.

90. The system of claim 78, wherein the system is configured to monitor deceleration of the z-actuator.

91. The system of claim 90, further comprising at least one sensor configured to detect a deceleration fault that is outside of an expected range or has repeatedly transitioned above one or more deceleration thresholds.

92. The system of claim 90, wherein the system is configured to prevent further use until inspection by an operator is performed, wherein, the treatment module is replaced or confirmation of no damage is provided by the operator.

93. The system according to claim 78, wherein the at least one coring element comprises at least three coring elements, wherein, the at least three coring elements are located at a separation distance of no more than 7 mm, 6 mm, 5 mm, or 4 mm.

94. The system of claim 93, wherein the at least one coring element comprises an outer diameter of between 0.0203″ and 0.0500″.

95. The system of claim 93, wherein the at least one coring element comprises an inner diameter of between 0.0103″ and 0.0207″.

96. The system as claimed in claim 78, wherein the treatment module is configured to detach and operably attach to the actuation assembly.

97. The system of claim 78, further comprising multiple treatment modules, wherein each treatment module comprises at least one coring element configured to remove a portion of skin tissue when the coring element is inserted into and withdrawn from the skin tissue, and wherein each treatment module is configured to be operably attached to the actuation assembly.

98. The system of claim 78, further comprising a receiving portion including a handle, wherein the treatment module is configured to operably attach to the receiving portion.

99. The system of claim 98, wherein at least one coring element is configured to be in a locked state when the treatment module is not attached to the receiving portion.

100. The system of claim 98, further comprising a sensor configured to produce a signal, wherein the system is configured to detect proper attachment of the treatment module to the receiving portion based on the signal.

101. The system of claim 100, wherein comprises a magnetic sensor.
